(19) United States
(12) Patent Application Publication
(10) Pub. No.: US 2021/0147514 A1
(43) Pub. Date: May 20, 2021

(54) ZIKA NEUTRALIZING ANTIBODY COMPOSITIONS AND METHODS OF USING THE SAME

(71) Applicant: Emergent BioSolutions Canada Inc., Winnipeg (CA)

(72) Inventor: Shantha KODIHALLI, Winnipeg (CA)

(21) Appl. No.: 17/254,479

(22) PCT Filed: Jun. 21, 2019
(86) PCT No.: PCT/IB2019/055275

§ 371 Date: Dec. 21, 2020

Related U.S. Application Data
(60) Provisional application No. 62/740,479, filed on Oct. 3, 2018.
(60) Provisional application No. 62/723,558, filed on Aug. 28, 2018.
(60) Provisional application No. 62/722,647, filed on Aug. 24, 2018.
(60) Provisional application No. 62/688,330, filed on Jun. 21, 2018.

(51) Int. Cl. C07K 16/10; A61P 31/14; C07K 16/06

(52) U.S. Cl. CPC C07K 16/10; A61K 2039/505; C07K 16/06; A61P 31/14

(57) ABSTRACT

The present disclosure is directed to compositions, including hyperimmune compositions, comprising Zika virus neutralizing antibodies and methods for using the same. For example, methods of treating, preventing, or reducing the risk of a Zika virus infection; methods for reducing viral load of a Zika virus; methods of eliciting an immune response against a Zika virus; methods of preventing or reducing the risk of transmission of a Zika virus infection from a subject; methods of treating, preventing, or reducing the risk of a Zika virus infection in an embryo or a fetus; methods for increasing antibody titer against a Zika virus infection; and methods of passive immunization against a Zika virus infection, and methods of preventing or reducing the severity or risk of microcephaly in a fetus are provided.

### CROSS-REFERENCE TO RELATED APPLICATIONS

This application claims priority to U.S. Provisional Application No. 62/740,479, filed Oct. 3, 2018, U.S. Provisional Application No. 62/723,558, filed Aug. 28, 2018, U.S. Provisional Application No. 62/722,647, filed Aug. 24, 2018, and U.S. Provisional Application No. 62/688,330, filed Jun. 21, 2018, each of which is herein incorporated by reference in its entirety.

### FIELD OF DISCLOSURE

The present disclosure relates to compositions, including hyperimmune compositions, comprising Zika virus neutralizing antibodies and methods for using the same.

### BACKGROUND

Zika virus (Zika) belongs to the genus Flavivirus within the family Flaviviridae. Many flaviviruses are significant human pathogens, including Zika, yellow fever, dengue virus, Japanese encephalitis, West Nile virus, and tick-borne encephalitis virus. Wong et al., EBioMedicine, 16:136-140, 2017. Zika is predominantly transmitted by mosquitoes but can also be transmitted through maternofetal route, sexual intercourse, blood transfusion, and organ transplantation. Musso et al., Clin Microbiol Rev, 29(3):487-524, 2016. While the majority of Zika infections are asymptomatic, symptoms of infection can include headaches, fever, lethargy, rash, conjunctivitis, myalgia, and arthralgia. In severe cases, infection can result in neurotropic Guillain-Barré syndrome and congenital microcephaly. Weaver et al., Antivir Res, 130:69-80, 2016.

Zika infection during pregnancy has been associated with a pattern of birth defects, sometimes called congenital Zika syndrome. Congenital Zika syndrome is unique to fetuses and infants infected with Zika virus before birth, and includes the following features: severe microcephaly in which the skull has partially collapsed; decreased brain tissue with a specific pattern of brain damage, including subcortical calcifications; damage to the back of the eye, including macular scarring and focal pigmentary retinal mottling; congenital contractures, such as clubfoot or arthrogryposis; and hypertonia restricting body movement soon after birth. Congenital Zika virus infection has also been associated with other abnormalities, including but not limited to brain atrophy and asymmetry, abnormally formed or absent brain structures, hydrocephalus, and neuronal migration disorders.

Like other Flavivirus, the Zika genome consists of a single-strand, positive-sense RNA of approximately 11,000 nucleotides. It contains a 5′ untranslated region (UTR), an open-reading frame (ORF), and a 3′ UTR. The single ORF encodes a long polyprotein which is processed into ten viral proteins, including three structural proteins (capsid (C), precursor membrane (prM), and envelope (E)) and seven non-structural proteins (NS1, NS2A, NS2B, NS3, NS4A, NS4B, and NS5). Lindenbach et al., Flaviviridae. In: Knipe, D. M., Howley, P. M. (Eds.), Fields Virology, 6th vol. 1. Lippincott William & Wilkins, Philadelphia, pp. 712-746, 2013.

Recent Zika virus epidemic in South Pacific and the Americas (e.g., French Polynesia in 2013 and in Brazil, Colombia, and Cape Verde in 2015) highlighted the potential severity of the virus. The exact global distribution of the virus worldwide is still not yet well understood. Mayer et al., Acta Prop, 166:155-163, 2017. Moreover, there are currently no approved treatments or vaccines available for Zika virus infection. Chen and Hamer, Ann Intern Med, 164(9):613-615, 2016.

### BRIEF SUMMARY

Provided herein is a method for treating, preventing, or reducing the risk of a Zika virus infection, comprising administering to a subject in need thereof an effective amount of a composition (e.g., a hyperimmune composition) comprising Zika virus neutralizing polyclonal antibodies, wherein the polyclonal antibodies are from pooled plasma and/or serum from mammalian donors.

Also provided herein is a method for reducing viral load of a Zika virus, comprising administering to a subject an effective amount of a composition (e.g., a hyperimmune composition) comprising Zika virus neutralizing polyclonal antibodies, wherein the polyclonal antibodies are from pooled plasma and/or serum from mammalian donors.

Also provided herein is a method for increasing antibody titers to a Zika virus, comprising administering to a subject an effective amount of a composition (e.g., a hyperimmune composition) comprising Zika virus neutralizing polyclonal antibodies, wherein the polyclonal antibodies are from pooled plasma and/or serum from mammalian donors.

Also provided herein is a method of eliciting an immune response against a Zika virus, comprising administering an effective amount of a composition (e.g., a hyperimmune composition) comprising Zika virus polyclonal antibodies to a subject, wherein the polyclonal antibodies are from pooled plasma and/or serum from mammalian donors.

Also provided herein is a method of passive immunization against a Zika virus, comprising administering an effective amount of a composition (e.g., a hyperimmune composition) comprising Zika virus polyclonal antibodies to a subject, wherein the polyclonal antibodies are from pooled plasma and/or serum from mammalian donors.

In some embodiments, the subject is pregnant, suspected of being pregnant, or trying to become pregnant with a fetus. In some embodiments, the subject is male.

Also provided herein is a method of preventing or reducing the risk of transmission of a Zika virus infection from a subject to an embryo, fetus, or infant, comprising administering to the subject an effective amount of a composition (e.g., a hyperimmune composition) comprising Zika virus neutralizing polyclonal antibodies, wherein the polyclonal antibodies are from pooled plasma and/or serum from mammalian donors.

Also provided herein is a method of preventing or reducing the risk of transmission of a Zika virus from a subject, comprising administering to the subject an effective amount of a composition (e.g., a hyperimmune composition) comprising Zika virus neutralizing polyclonal antibodies, wherein the polyclonal antibodies are from pooled plasma and/or serum from mammalian donors, and wherein the subject is trying to become pregnant.

Also provided herein is a method of treating, preventing, or reducing the risk of a Zika virus infection in an embryo or a fetus, comprising administering an effective amount of a composition (e.g., a hyperimmune composition) comprising Zika virus neutralizing polyclonal antibodies to a subject pregnant with the embryo or the fetus, wherein the polyclonal antibodies are from pooled plasma and/or serum from mammalian donors.

Also provided herein is a method of preventing or reducing the severity or risk of microcephaly in a fetus, comprising administering to a pregnant subject carrying the fetus an effective amount of a composition (e.g., a hyperimmune composition) comprising Zika virus neutralizing polyclonal antibodies, wherein the polyclonal antibodies are from pooled plasma and/or serum from mammalian donors.

In some embodiments, the mammalian donors are human.

In some embodiments, the polyclonal antibodies are from pooled plasma of one or more human donors.

In some embodiments, the mammalian donors were infected with Zika virus prior to pooling plasma and/or serum.

In some embodiments, the mammalian donors were vaccinated with Zika vaccine prior to pooling plasma and/or serum.

In some embodiments, the mammalian donors have elevated levels of anti-Zika virus antibodies.

In some embodiments, the mammalian donors have elevated levels of antibodies against a Zika Non-Structural protein 1 (anti-NS1 antibody) and/or a Zika Envelope protein (anti-E-protein antibody).

In some embodiments, the polyclonal antibodies comprise IgG antibodies.

In some embodiments, the IgG antibodies are greater than 95% of the antibody content of the composition.

In some embodiments, the effective amount is sufficient to provide a Zika virus antigen-specific immune response in the subject.

In some embodiments, the effective amount is sufficient to neutralize the Zika virus in the subject.

In some embodiments, the subject is pregnant and transmission of Zika virus from the pregnant subject to the embryo or the fetus is prevented, reduced or eliminated.

In some embodiments, the subject is pregnant and the effective amount is sufficient to provide a Zika virus antigen-specific immune response in the fetus.

In some embodiments, the method comprises passive immunization of the fetus.

In some embodiments, the subject and fetus are human.

In some embodiments, the subject is in the first trimester, second trimester or third trimester of pregnancy.

In some embodiments, the subject is in the late stage of the first trimester or early stage of the second trimester of pregnancy.

In some embodiments, the risk of miscarriage and/or stillbirth is reduced.

In some embodiments, the subject has been bitten by a mosquito suspected of harboring the Zika virus, lives in an area that has a Zika virus outbreak, is visiting or has visited an area that has a Zika virus outbreak, is immunocompromised, is suspected of having been exposed to a person harboring the Zika virus, has come into physical contact or close physical proximity with an infected individual, is a hospital employee, and/or lives in or is visiting a country or region known to have mosquitoes harboring the Zika virus.

In some embodiments, the composition (e.g., the hyperimmune composition) is administered to the subject before the subject has been infected with the Zika virus, after the subject has been infected with the Zika virus, or after the subject has been exposed to or is suspected of having been exposed to the Zika virus and before the Zika virus infection can be detected.

In some embodiments, the subject has been diagnosed with having or is suspected of having African lineage Zika virus strain, Asian lineage Zika virus strain, Brazil lineage virus strain, or Puerto Rico lineage virus strain.

In some embodiments, the subject has been diagnosed with having or is suspected of having Zika virus strain MR 766, FLR, Brazil-ZKV2015, or PRVABC59.

In some embodiments, the administration treats, prevents or reduces the risk of one or more symptoms associated with Zika virus infection.

In some embodiments, the one or more symptoms associated with the Zika virus infection comprise a fever, rash, headache, joint pain, conjunctivitis, or muscle pain.

In some embodiments, the administration is intravenous, intramuscular, subcutaneous, or intrauterinal.

In some embodiments, the composition (e.g., the hyperimmune composition) is administered as at least one dose of about 50 mg/kg to about 400 mg/kg.

In some embodiments, the duration of Zika viremia in the subject and/or fetus is shortened.

In some embodiments, the Zika viral load in the blood and/or a tissue of the subject and/or fetus is prevented or decreased.

In some embodiments, the Zika viral load is decreased by at least 25%, at least 50%, at least 75%, at least 85%, at least 90%, at least 95%, at least 99%, or 100%.

In some embodiments, the Zika viral load in the blood is decreased in the subject.

In some embodiments, the Zika viral load in the blood is prevented or decreased in the fetus.

In some embodiments, the Zika viral load in a tissue in the subject is decreased.

In some embodiments, the Zika viral load in a tissue in the fetus is prevented or decreased.

In some embodiments, the tissue is selected from the group consisting of brain, dura mater, spinal cord, sciatic nerve, cochlea, cerebrum, cerebellum, aqueous humor, optic nerve, sclera, cornea, retina, pericardium, heart, aorta, lung, seminal vesicle, prostate/uterus, testis, ovary, articular cartilage, adipose tissue-omentus, epidermis/dermis of abdomen, muscle-quadriceps, bone marrow, tonsil, spleen, thymus, lymph nodes, gastric contents, esophagus, stomach, duodenum, jejunum, ileum, cecum, colon, bile aspirate, liver, meconium, tongue, urinary bladder, kidney, urine, thyroid, adrenal gland, pituitary, pancreas, fetal blood, placental disk, uterus, decidua, amniotic/chorionic membrane, amniotic fluid, umbilical cord, cord blood, or any combination thereof.

### BRIEF DESCRIPTION OF THE DRAWINGS

FIG. 1 shows survival data for interferon-alpha/beta receptor alpha chain knock-out (Ifnar1−/−) mice treated with the indicated dose levels of Zika virus (ZIKV) polyclonal antibodies (ZIKV-IG) administered at 1 hour pre-exposure or 24 hours post-exposure to a lethal challenge of Zika virus. These results show that pre- and post-exposure treatment with ZIKA-IG provided full protection and all but one treated mouse made a full recovery by the end of the study (21 days) compared to 0% survival in the vehicle control group.

FIG. 2 shows body weight data of Ifnar1−/− mice treated with various dose levels of ZIKV-IG administered at 1 hour pre-exposure or 24 hours post-exposure to a lethal challenge of Zika virus. These results show that body weight of all mice treated with ZIKV-IG increased, while the body weight of all control mice decreased.

FIG. 3 shows clinical scores of Ifnar1−/− mice treated with various dose levels of ZIKV-IG administered at 1 hour pre-exposure or 24 hours post-exposure to a lethal challenge of Zika virus. These results show that the clinical score of control treated mice progressively increased, while the clinical score of all ZIKV-IG treated mice at most reached 3 and declined prior to a full recovery, with the exception of one mouse that developed hydrocephaly from the group treated pre-exposure to ZIKV at 50 mg/kg ZIKV-IG.

FIG. 4 shows survival data of Ifnar1−/− mice treated with the indicated dose levels of ZIKV-IG administered at 24 hours post-exposure to a lethal challenge of Zika virus. These results show that mice treated with 50 mg/kg and 10 mg/kg ZIKV-IG had significantly higher survival compared to control mice.

FIG. 5 shows body weight data of Ifnar1−/− mice treated with the indicated dose levels of ZIKV-IG administered at 24 hours post-exposure to a lethal challenge of Zika virus. These results show that mice treated with 50 mg/kg ZIKV-IG had significant improvement in body weight compared to control mice and indicate a dose-related treatment effect.

FIG. 6 shows clinical health scores of Ifnar1−/− mice treated with the indicated dose levels of ZIKV-IG administered at 24 hours post-exposure to a lethal challenge of Zika virus. These results show that clinical scores at day 8 in the vehicle control (A) and 0.5 mg/kg-treated (E) groups were 3, but clinical scores in the 50 mg/kg ZIKV-IG treated group were less than 2, suggesting no disease progression in this group.

FIG. 7A, FIG. 7B, FIG. 7C, and FIG. 7D show viral load data from ZIKV-infected Ifnar1−/− mice treated with PBS (control group) or 50, 10, 2 and 0.5 mg/kg of ZIKV-IG 24 hours after infection. Following treatment, brain (FIG. 7A and FIG. 7B) and sciatic nerve (FIG. 7C and FIG. 7D) were harvested on days 3 and 7, and viral load was determined using quantitative reverse transcriptase-polymerase chain reaction (qRT-PCR) analysis. Viral RNA levels in these tissues were expressed of log₁₀ genome copies per 18S copies (log₁₀ ZIKV/18S). The dotted line represents the limit of detection (LOD). **=Significant difference (p≤0.05) observed between ZIKV-IG treated groups and control group.

FIG. 8A, FIG. 8B, FIG. 8C, and FIG. 8D show viral load data from ZIKV-infected Ifnar1−/− mice treated with PBS (control group) or 50, 10, 2 and 0.5 mg/kg of ZIKV-IG 24 hours after infection. Following treatment, kidney (FIG. 8A and FIG. 8B) and serum (FIG. 8C and FIG. 8D) were harvested on days 3 and 7, and viral load was determined using qRT-PCR analysis. Viral RNA level in kidney was expressed of logo genome copies per 18S copies (log₁₀ ZIKV/18S). Viral RNA level in serum was expressed as log₁₀ genome copies per mL. The dotted line represents the limit of detection (LOD). **=Significant difference (p≤0.05) observed between ZIKV-IG treated groups and control group.

FIG. 9A, FIG. 9B, FIG. 9C, and FIG. 9D show viral load data from ZIKV-infected Ifnar1−/− mice treated with PBS (control group) or 50, 10, 2 and 0.5 mg/kg of ZIKV-IG 24 hours after infection. Following treatment, spleen (FIG. 9A and FIG. 9B) and liver (FIG. 9C and FIG. 9D) were harvested on days 3 and 7, and viral load was determined using qRT-PCR analysis. Viral RNA level in these tissues were expressed of log₁₀ genome copies per 18S copies (log₁₀ ZIKV/18S). The dotted line represents the limit of detection (LOD). **=Significant difference (p≤0.05) observed between ZIKV-IG treated groups and control group.

FIG. 10A, FIG. 10B, FIG. 10C, and FIG. 10D show viral load data from ZIKV-infected Ifnar1−/− mice treated with PBS (control group) or 50, 10, 2 and 0.5 mg/kg of ZIKV-IG 24 hours after infection. Following treatment, testes (FIG. 10A and FIG. 10B) and ovary (FIG. 10C and FIG. 10D) were harvested on days 3 and 7, and viral load was determined using qRT-PCR analysis. Viral RNA level in these tissues were expressed of log₁₀ genome copies per 18S copies (log₁₀ ZIKV/18S). The dotted line represents the limit of detection (LOD). **=Significant difference (p≤0.05) observed between ZIKV-IG treated groups and control group.

FIG. 11A, FIG. 11B, FIG. 11C, and FIG. 11D show focus forming assay (FFA) analysis of viral load from ZIKV-infected Ifnar1−/− mice treated with PBS (control group) or 50, 10, 2 and 0.5 mg/kg of ZIKV-IG 24 hours after infection. Following treatment, brain (FIG. 11A and FIG. 11B) and sciatic nerve (FIG. 11C and FIG. 11D) were harvested on days 3 and 7, and viral load was determined using FFA. Viral load in brain was reported as log₁₀ focus forming units (FFU)/g of tissue and log₁₀ FFU/tissue for sciatic nerve. The dotted line represents the limit of detection (LOD). **=Significant difference (p≤0.05) observed between ZIKV-IG treated groups and control group.

FIG. 12A, FIG. 12B, FIG. 12C, and FIG. 12D show FFA analysis of viral load from ZIKV-infected Ifnar1−/− mice treated with PBS (control group) or 50, 10, 2 and 0.5 mg/kg of ZIKV-IG 24 hours after infection. Following treatment, kidney (FIG. 12A and FIG. 12B) and serum (FIG. 12C and FIG. 12D) were harvested on days 3 and 7, and viral load was determined using FFA. Viral load in kidney was reported as log₁₀ FFU/g of tissue and log₁₀ FFU/ml for serum. The dotted line represents the limit of detection (LOD). **=Significant difference (p≤0.05) observed between ZIKV-IG treated groups and control group.

FIG. 13A, FIG. 13B, FIG. 13C, and FIG. 13D show FFA analysis of viral load from ZIKV-infected Ifnar1−/− mice treated with PBS (control group) or 50, 10, 2 and 0.5 mg/kg of ZIKV-IG 24 hours after infection. Following treatment, spleen (FIG. 13A and FIG. 13B) and liver (FIG. 13C and FIG. 13D) were harvested on days 3 and 7, and viral load was determined using FFA. Viral load in spleen and liver was reported as log₁₀ FFU/g of tissue. The dotted line represents the limit of detection (LOD). **=Significant difference (p≤0.05) observed between ZIKV-IG treated groups and control group.

FIG. 14A, FIG. 14B, FIG. 14C, and FIG. 14D show FFA analysis of viral load from ZIKV-infected Ifnar1−/− mice treated with PBS (control group) or 50, 10, 2 and 0.5 mg/kg of ZIKV-IG 24 hours after infection. Following treatment, testes (FIG. 14A and FIG. 14B) and ovaries (FIG. 14C and FIG. 14D) were harvested on days 3 and 7, and viral load was determined using FFA. Viral load in testes was reported as log₁₀ FFU/g of tissue and log₁₀ FFU/tissue for ovaries. The dotted line represents the limit of detection (LOD). **=Significant difference (p≤0.05) observed between ZIKV-IG treated groups and control group.

FIG. 15 shows microscopic lesion severity in the brain, liver and spleen of PBS control-treated and 50 mg/kg ZIKV-IG-treated Ifnar1−/− mice.

FIG. 16A, FIG. 16B, and FIG. 16C show the results of immunohistochemistry assays performed on brain and liver tissues collected from 4 groups of mice sacrificed on day 7 and 21. The 4 groups of mice were treated as follows: Study Group A=Control; Study Group C=0.5 mg/kg ZIKV-IG; Study Group B1=50 mg/kg ZIKV-IG (Day 7 harvest); and Study Group B1=50 mg/kg ZIKV-IG (Day 21 harvest). FIG. 16A shows positive cell density results from brain tissue of the mice. FIG. 16B shows positive cell density results from liver tissue of the mice. The data is presented as mean SEM ZIKV positive cell densities in brain and liver tissues. FIG. 16C shows immunohistochemical tissue staining images from brain and liver (LIV) of PBS control-treated and 50 mg/kg ZIKV-IG-treated groups (upper and lower panels, respectively).

FIG. 17A, FIG. 17B, and FIG. 17C show focus forming reduction neutralization (FRNT) test results showing in vitro neutralization potency of pilot and clinical lots of ZIKV-IG against (FIG. 17A) Zika virus (ZIKV), (FIG. 17B) Dengue virus type 2 (DENV2), and (FIG. 17C) Dengue virus type 3 (DENV3). Virus and antibody were incubated at 37° C. for 1 hour then incubated on Vero cells to determine neutralization potential. Data shown are the mean and standard deviation of 2-3 independent experiments completed in duplicate. These results compare the neutralization potency of both ZIKV-IG lots to DENV2 and DENV3.

FIG. 18A and FIG. 18B show viral load measurements over time from ZIKV-infected pregnant female rhesus macaques treated with ZIKV-IG (“ZIKV-Ig”; 279087, 518832, and 581937) compared to viral load measurements from historical controls (“historical controls”; 484880, 795784, and 527453) or placebo controls (“placebo”; 558656, 568603, and 636528). FIG. 18C additionally shows viral load measurements over time from another ZIKV-infected pregnant female rhesus macaques treated with ZIKV-IG (“ZIKV-Ig”; 240385). FIG. 18D shows viral load measurements over time from ZIKV-infected pregnant female rhesus macaques treated with ZIKV-IG (“ZIKV-Ig”; 240385, 518832, 279087, and 581937) compared to viral load measurements from historical controls (“historical”; 244667, 480311, 699597, 810356, 930221, 484880, 795784, and 527453) or placebo controls (“placebo”; 558656, 636528, 568603, and 240973).

FIG. 19A, FIG. 19B, and FIG. 19C show the results of plaque reduction neutralization test (PRNT) assays performed using serum collected from ZIKV-infected pregnant female rhesus macaques treated with ZIKV-IG. The serum was collected at the specified day post infection (dpi), specifically, on day 1 at 1 and 6 hours after the first human immunoglobulin (HIG) injection, days 2-5, day 5 at 1 and 6 hours after the second HIG injection and days 6, 7, 16, 20 and 27. Serum was serially diluted, combined with 200 plaque forming units (PFU) of ZIKV (PRVABC59), and plated with Vero cells. After incubation of the virus and serum with the cells, the cultures were overlaid with agar and stained with Neutral red at day 4 or 5. Plaques were counted daily until they no longer increased in frequency and PRNT₅₀ (EC₅₀) and PRNT₉₀ (EC₉₀) values were assessed. FIG. 19A shows EC90 and 50 for ZIKV-infected pregnant female rhesus macaque (581937) at each time point; and FIG. 19B shows % Plaque Reduction for ZIKV-infected pregnant female rhesus macaque (581937) at 0 dpi; 1 dpi, 1 hour post-treatment; 1 dpi, 6 hours post-treatment; and 1 dpi, pre-treatment compared to positive control. FIG. 19C shows EC90 and 50 for ZIKV-infected pregnant female rhesus macaque (279087) at 1 dpi, 1 hour post-treatment and 1 dpi, 6 hours post-treatment.

FIG. 20 shows % Plaque Reduction for ZIKA-IG compared to placebo and positive control; and preliminary neutralization titer values (PRNT₉₀ and PRNT₅₀) prior to administration of 50 mg/kg ZIKV-IG, placebo or control.

FIG. 21A, FIG. 21B, and FIG. 21C show serum ZIKV-IG titer values (PRNT₉₀ and PRNT₅₀) over time after ZIKV-IG administration from ZIKV-infected pregnant female rhesus macaques. FIG. 21A shows the EC90 and 50 after ZIKV-IG administration to a ZIKV-infected pregnant female rhesus macaque (581937) from serum taken prior to infection (0 dpi), 1 day post-infection prior to pretreatment, 1 day post-infection at 1 and 6 hours after the first injection of 50 mg/kg ZIKV-IG, days 2-5, day 5 at 1 and 6 hours after the second injection of 50 mg/kg ZIKV-IG, and at days 6 and 7. FIG. 21B shows the PRNT₉₀ Titer after ZIKV-IG administration to ZIKV-infected pregnant female rhesus macaques (581937 and 279087) from serum taken 1 day post-infection at 1 and 6 hours after the first injection, days 2-5, day 5 at 1 and 6 hours after the second injection, and days 6, 7, 16, 20, 27, 34, 41, and 55 compared to historical controls (“Historical”) at day 28 post-infection. FIG. 21C shows the PRNT₉₀ Titer after ZIKV-IG administration to ZIKV-infected pregnant female rhesus macaques (581937 and 279087) from serum taken 1 day post-infection at 1 and 6 hours after the first injection, days 2-5, day 5 at 1 and 6 hours after the second injection, and days 6, 7, 16, 20, 27, 34, 41, and 55 compared to ZIKV-infected pregnant female rhesus macaque (240385) and placebo controls (“placebo”; 636528 and 558656) at day 27 post-infection.

FIG. 22A, FIG. 22B, FIG. 22C, FIG. 22D, FIG. 22E, FIG. 22F, FIG. 22G, and FIG. 22H show preliminary results for antibody concentrations measured by ELISA and estimated half-life of human ZIKV-IG. FIG. 22A shows human IG (HIG) concentration in samples from ZIKV-infected pregnant female rhesus macaque treated with ZIKV-IG(581937 and 279087) or non-specific human IG control (636528). FIG. 22B shows human IG concentration in samples from ZIKV-infected pregnant female rhesus macaque treated with ZIKV-IG (581937, 279087, 518832, and 240385) over time post-infection. FIG. 22C shows natural antibody response in ZIKV-infected pregnant female rhesus macaque treated with ZIKV-IG (581937 and 279087) or non-specific human IG control (636528). FIG. 22D, FIG. 22E, FIG. 22F, and FIG. 22G show estimated half-life calculations for peak 1 and peak 2 of human ZIKV-IG in ZIKV-infected pregnant female rhesus macaque treated with ZIKV-IG (581937 and 279087). FIG. 22H shows rhesus IG concentration in samples from ZIKV-infected pregnant female rhesus macaque treated with ZIKV-IG (581937, 279087, 518832, and 240385) over time post-infection.

FIG. 23 shows viral load data summary from ZIKV-infected pregnant female rhesus macaque treated with ZIKV-IG (581937/729723 and 279087/608886) or placebo-IG treated (636528/107099 and 558656/572098). Following treatment, maternal, maternal/fetal, or fetal tissues were harvested and viral load was determined. Viral RNA level in different tissues was expressed as “copies vRNA/mg tissue.”

### DETAILED DESCRIPTION

The present application provides methods of treating, preventing or reducing the risk of a Zika virus infection, and methods for reducing viral load of Zika virus in a subject. The present application also provides methods of passive immunization and eliciting an immune response against a Zika virus and methods for preventing or reducing the severity or risk of Zika virus associated birth defects such as microcephaly, brain damage, and eye damage.

The present disclosure provides a method for treating, preventing or reducing the risk of a Zika virus infection, comprising administering to a subject in need thereof a composition (e.g., a hyperimmune composition) comprising Zika virus neutralizing antibodies. In some embodiments, the antibodies are polyclonal antibodies. In some embodiments, the polyclonal antibodies are from pooled plasma and/or serum from mammalian donors. In some embodiments, the composition is a hyperimmune composition.

The present disclosure also provides a method for reducing viral load of a Zika virus in a bodily fluid, tissue or cell of a subject, comprising administering to the subject an effective amount of a composition (e.g., a hyperimmune composition) comprising Zika virus neutralizing antibodies. In some embodiments, the antibodies are polyclonal antibodies. In some embodiments, the polyclonal antibodies are from pooled plasma and/or serum from mammalian donors. In some embodiments, the composition is a hyperimmune composition.

The present disclosure also provides a method of eliciting an immune response against a Zika virus, comprising administering an effective amount of a composition (e.g., a hyperimmune composition) comprising Zika virus antibodies to a subject. In some embodiments, the antibodies are polyclonal antibodies. In some embodiments, the polyclonal antibodies are from pooled plasma and/or serum from mammalian donors. In some embodiments, the composition is a hyperimmune composition.

The present disclosure also provides a method passive immunization against a Zika virus, comprising administering an effective amount of a composition (e.g., a hyperimmune composition) comprising Zika virus antibodies to a subject. In some embodiments, the antibodies are polyclonal antibodies. In some embodiments, the polyclonal antibodies are from pooled plasma and/or serum from mammalian donors. In some embodiments, the composition is a hyperimmune composition.

The present disclosure also provides a method of preventing or reducing the risk of transmission of a Zika virus infection from a subject to an embryo, fetus or infant, comprising administering to the subject an effective amount of a composition (e.g., a hyperimmune composition) comprising Zika virus neutralizing antibodies. In some embodiments, the antibodies are polyclonal antibodies. In some embodiments, the polyclonal antibodies are from pooled plasma and/or serum from mammalian donors. In some embodiments, the composition is a hyperimmune composition. In some embodiments, the transmission is vertical transmission.

The present disclosure also provides a method of preventing or reducing the risk of transmission of a Zika virus infection from a subject, comprising administering to the subject an effective amount of a composition (e.g., a hyperimmune composition) comprising Zika virus neutralizing antibodies. In some embodiments, the antibodies are polyclonal antibodies. In some embodiments, the polyclonal antibodies are from pooled plasma and/or serum from mammalian donors. In some embodiments, the composition is a hyperimmune composition. In some embodiments, the subject is trying to become pregnant. In some embodiments, the transmission is from a male subject to a female subject. In some embodiments the transmission is from a female subject to a male subject. In some embodiments, the transmission is vertical transmission. In some embodiments, the transmission is from a female subject to an embryo, a fetus, or an infant.

The present disclosure also provides a method of treating, preventing, or reducing the risk of a Zika virus infection in an embryo or a fetus, comprising administering an effective amount of a composition (e.g., a hyperimmune composition) comprising Zika virus neutralizing antibodies to a subject pregnant with the embryo or the fetus. In some embodiments, the antibodies are polyclonal antibodies. In some embodiments, the polyclonal antibodies are from pooled plasma and/or serum from mammalian donors. In some embodiments, the composition is a hyperimmune composition.

The present disclosure also provides a method of preventing or reducing the severity or risk of microcephaly in a fetus, comprising administering to a pregnant subject carrying the fetus an effective amount of a composition (e.g., a hyperimmune composition) comprising Zika virus neutralizing antibodies. In some embodiments, the antibodies are polyclonal antibodies. In some embodiments, the polyclonal antibodies are from pooled plasma and/or serum from mammalian donors. In some embodiments, the composition is a hyperimmune composition.

### Definitions

In order that the present disclosure can be more readily understood, certain terms are first defined. As used in this application, except as otherwise expressly provided herein, each of the following terms shall have the meaning set forth below. Additional definitions are set forth throughout the application.

As used herein, “a” or “an” means one or more unless otherwise specified.

As used herein, “and/or” is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the term “about” is understood as within a range of normal tolerance in the art and not more than 10% of a stated value. By way of example only, about 50 means from 45 to 55 including all values in between. As used herein, the phrase “about” a specific value also includes the specific value, for example, about 50 includes 50.

The terms “antibody”, “antibodies”, “immunoglobulin”, “immune globulin”, “immune globulins”, and “immunoglobulins” can be used interchangeably herein and refer to a molecule with an antigen binding site that specifically binds an antigen. The terms as used herein include whole antibodies and any antigen binding fragments (i.e., “antigen-binding fragments”) or single chains thereof. An “antibody” refers, in one embodiment, to a glycoprotein comprising at least two heavy (H) chains and two light (L) chains inter-connected by disulfide bonds, or an antigen-binding fragment thereof. In another embodiment, an “antibody” refers to a single chain antibody comprising a single variable domain, e.g., VHH domain. Each heavy chain is comprised of a heavy chain variable region (abbreviated herein as VH) and a heavy chain constant region. In certain naturally-occurring antibodies, the heavy chain constant region is comprised of three domains, CH1, CH2 and CH3. In certain naturally-occurring antibodies, each light chain is comprised of a light chain variable region (abbreviated herein as VL) and a light chain constant region. The light chain constant region is comprised of one domain, CL.

As used herein, the term “polyclonal antibodies” refers to a mixture of immunoglobulins secreted by different B cell lineages that react against a specific antigen, but identify different epitopes on the antigen (e.g., a Zika virus antigen).

As used herein, the terms “neutralizing antibody” or “neutralizing antibodies” refer to antibodies that bind a target (e.g., a Zika virus antigen) where such binding results in neutralizing the biological effect of the target.

As used herein, the terms “antigen” and “immunogen” refer to any substance that is capable of inducing an immune response. An antigen may be whole cell (e.g., bacterial cell), virus, fungus, or an antigenic portion or component thereof. Non-limiting examples of antigens for the present disclosure include a Zika virus Envelope protein or a fragment or a variant thereof, or a Zika virus Non-structural 1 protein or a fragment or a variant thereof.

As used herein, the term “epitope” designates a particular molecular surface feature of an antigen, for example a fragment of an antigen, which is capable of being bound by at least one antibody. Antigens usually present several surface features that can act as points of interaction for specific antibodies. Any such distinct molecular feature constitutes an epitope. On a molecular level, an epitope therefore corresponds to a particular molecular surface feature of an antigen (for example a fragment of an antigen) which is recognized and bound by a specific antibody.

As used herein, the term “viral infection” refers to a diseased state in which a virus (e.g., a Zika virus) invades a cell and uses the cell's machinery to multiply or replicate, ultimately resulting in the release of new viral particles. This release results in the infection of other cells by the newly produced particles. Latent infection by certain viruses is also a possible result of viral infection.

As used herein, the term “viral load” refers to the quantity of virus in a given volume. In some embodiments, this term refers to a measurement of the amount of a virus in an organism, typically in the bloodstream, usually stated in virus particles per milliliter.

As used herein, the term “flavivirus” refers to viruses belonging to the genus Flavivirus of the family Flaviviridae. According to virus taxonomy, about 50 viruses including, e.g., Zika, Hepatitis C (HCV), Yellow Fever, Dengue, Japanese Encephalitis, West Nile, and related flaviviruses are members of this genus. The viruses belonging to the genus Flavivirus are referred to herein as flaviviruses. Currently, these viruses are predominantly in East, Southeast and South Asia and Africa, although they may be found in other parts of the world, such as South America.

As used herein, the term “Zika virus” comprises any Zika virus, irrespective of strain or origin. In some embodiments, the term relates to a Zika virus from an African or an Asian lineage. In other embodiments, the term “Zika virus” comprises a Zika virus strain selected from the group consisting of (i) strain PLCal_ZV-Thailand (GenBank Accession No. KF993678); (ii) strain PRVABC59-Puerto Rico (GenBank Accession No. KU501215); (iii) strain IbH_30656-Nigeria (GenBank Accession No. HQ234500); (iv) strain MR 766-Uganda (GenBank Accession No. LC002520); (v) strain FSS13025-Cambodia (GenBank Accession No. JN860885); (vi) strain SPH2015-Brazil (GeneBank Accession No. KU321639); (vii) strain Brazil_ZKV2015 (GenBank Accession No. KU497555); and (viii) strain H/PF/2013-Polynesia (GenBank Accession No. KJ776791).

As used herein, the terms “treat,” “treating,” and “treatment” refer to administering a therapy in an amount, manner, or mode effective to improve a condition, symptom, or parameter associated with a disease or disorder (e.g., Zika virus infection). Thus, “treating” a Zika virus infection means inhibiting or preventing the replication of the virus, inhibiting, or preventing viral transmission, and/or ameliorating, alleviating, or otherwise improving the symptoms of a disease or condition caused by or associated with the virus. In some embodiments, the treatment can be considered therapeutic if there is a reduction in viral load, and/or a decrease in mortality and/or morbidity.

As used herein, the term “reducing the risk of a Zika virus infection” refers to decreasing the likelihood or probability of developing a disease, disorder, or symptom associated with a Zika virus infection in a subject, wherein the subject is, for example a subject who is at risk for developing such a disease, disorder, or symptom.

As used herein, the terms “preventing” and “prevention” as used with the methods of the invention described herein refer to activities designed to protect patients or other members (e.g., individuals) of the public from actual or potential health threats and their harmful consequences.

As used herein, the term “vertical transmission” refers to delivery of a pathogen (e.g., a virus) from a mother to a child. Vertical transmission includes, but is not limited to, delivery from a mother to a fetus, from a mother to an embryo, from a mother to an infant during pregnancy, or from a mother to an infant during childbirth.

As used herein, the term “effective amount” refers to an amount of a therapeutic agent or a composition comprising a therapeutic agent (e.g., a hyperimmune composition), alone or in combination with another therapeutic agent, effective to treat or reduce symptoms, or reduce the risk, potential, possibility or occurrence of a disease or disorder (e.g., a Zika virus infection) in a subject or an embryo, fetus or infant carried by a subject. An effective amount can include an amount of a therapeutic agent or a composition comprising a therapeutic agent (e.g., a hyperimmune composition), alone or in combination with another therapeutic agent, that provides some improvement or benefit to a subject having or at risk of having a Zika virus infection or some improvement or benefit to an embryo, fetus or infant carried by a subject having or at risk of having a Zika virus infection.

As used herein, “administering” refers to the physical introduction of a therapeutic agent or a composition comprising a therapeutic agent (e.g., a hyperimmune composition) to a subject or an embryo, fetus or infant carried by a subject, using any of the various methods and delivery systems known to those skilled in the art. The different routes of administration for antibodies described herein include intravenous, intraperitoneal, intramuscular, subcutaneous, intrauterinal, spinal or other parenteral routes of administration, for example, by injection or infusion. As used herein, the term “parenteral administration” means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcutaneous, subcuticular, intraarticular, subcapsular, subarachnoid, intraventricle, intravitreal, epidural, and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

As used herein, the term “vaccine” refers to a prophylactic or therapeutic material providing at least one antigen, preferably an immunogen. The antigen or immunogen may be derived from any material that is suitable for vaccination. For example, the antigen or immunogen may be derived from a pathogen, such as from bacteria or virus particles etc., or from a tumor or cancerous tissue. The vaccine antigen or immunogen stimulates the body's adaptive immune system to provide an adaptive immune response.

As used herein, the term “immunized” refers to being sufficiently vaccinated to achieve a protective immune response.

As used herein, the term “hyperimmune” refers to a state of having an elevated level of antibodies to a target, e.g., against a Zika virus, compared to a reference level (e.g., level of anti-Zika virus antibodies in normal source donor comprising non-specific antibodies). In some embodiments, the elevated level of antibodies to a target is generated from exposure to the target virus. In another embodiment, the elevated level of antibodies is generated from donor stimulation (e.g., administration of a vaccine to the target). In another embodiment, the elevated level of antibodies is generated by purifying an immunoglobulin source. In some embodiments, the antibodies disclosed herein are immune globulins.

As used herein, the term “hyperimmune composition” (e.g., Zika virus hyperimmune composition) refer to a composition comprising an elevated level of antibodies, e.g., polyclonal antibodies, to one or more specific antigens, which is obtained from plasma and/or serum. In some embodiments, the hyperimmune composition is enriched with antibodies specific to one or more particular epitopes of a Zika virus (e.g., anti-NS1 and/or anti-E-protein antibodies). In some embodiments, the hyperimmune compositions disclosed herein are prepared from plasma and/or serum obtained from an individual (e.g., human, animal or convalescent donor) or pool of individuals (e.g., donors) with elevated levels of anti-Zika virus antibodies. In some embodiments, the individual or pool of individuals disclosed herein have elevated levels of anti-Zika virus antibodies due to previous exposure to a Zika virus antigen (e.g., an individual or pool of individuals previously infected with a Zika virus). In some embodiments, the individual or pool of individuals disclosed herein have elevated levels of anti-Zika virus antibodies due to intentional stimulation of the immune response (e.g., administration of a Zika vaccine). In some embodiments, the hyperimmune composition contains purified immunoglobulins derived from such individuals or pools of individuals. In some embodiments, the antibodies disclosed herein are immune globulins. In some embodiments, the hyperimmune composition comprises IgG antibodies.

As used herein, the term “hyperimmunization” or “hyperimmunized” refer to a state of immunity that is greater than normal (e.g., non-infected subjects, e.g., healthy subjects) and results in a higher titer than normal number of antibodies to an antigen. In some embodiments, hyperimmunization can be the result of a previous infection with the Zika virus, such that the individual or pool of individuals have higher titer of certain antibodies against the Zika virus, e.g., Envelope protein and/or the NS1 protein, compared to an individual or pool of individuals who have never been infected with a Zika virus. In some embodiments, hyperimmunization can involve the repeated administration of a single antigen (e.g., Zika virus Envelope protein or the NS1 protein) or multiple antigens of a given virus (e.g., both Zika virus Envelope and the NS1 proteins) to one or more subjects to generate an enhanced immune response (e.g., higher titer of antibodies against Zika virus Envelope protein and/or NS1 protein compared to a subject not exposed to the antigen).

As used herein, the term “passive immunization” refers to conferral of immunity by the administration, by any route, of exogenously produced immune molecules (e.g., antibodies) into a subject. Passive immunization differs from “active” immunization, where immunity is obtained by introduction of an immunogen into an individual to elicit an immune response.

As used herein, the terms “pooled plasma,” “pooled plasma samples,” and “pooled plasma composition” refer to a mixture of two or more plasma samples from one or more donors and/or a composition prepared from the same (e.g., an immunoglobulin composition). In some embodiments, the plasma samples are obtained from a single donor. In some embodiments, the plasma samples are obtained from multiple donors. Elevated titer of a particular antibody or set of antibodies in pooled plasma reflects the elevated titers of the antibody samples that make up the pooled plasma. For example, plasma samples can be obtained from donors or subjects that have been vaccinated (e.g., with a vaccine) or donors or subjects that have high titers of antibodies to a Zika virus antigen (e.g., after a Zika virus infection) as compared to the antibody level(s) found in a population of subjects never infected with Zika virus or the population as a whole. Upon pooling of the plasma samples, a pooled plasma composition is produced (e.g., that has an elevated titer of antibodies specific to a particular antigen). Pooled plasma compositions can be used to prepare immunoglobulin (e.g., that is subsequently administered to a subject) via methods known in the art (e.g., fractionation, purification, isolation, etc.). The present disclosure provides that pooled plasma compositions, pooled serum compositions, and immunoglobulin compositions prepared from same can be administered to a subject to provide prophylactic and/or therapeutic benefits to the subject or an embryo, fetus or infant carried by a subject. Accordingly, the term pooled plasma composition or pooled serum composition can refer to immunoglobulin prepared from pooled plasma or pooled serum samples, respectively.

As used herein, the terms “subject” or “individual”, used interchangeably herein, refer to any subject, particularly a mammalian subject, particularly humans. Other subjects can include non-human primates, cattle, dogs, cats, guinea pigs, rabbits, rats, mice, horses, goats, sheep, and so on. In some embodiments, the subject can be a pregnant mammal, and in particular embodiments, a pregnant human female. In some embodiments, the subject is male. In some embodiments, the subject is an infant. In some embodiments, the subject is a newborn. In some embodiments, the subject is a patient, for whom prophylaxis or therapy is desired. In some embodiments, the subject is a donor. In some embodiments, the terms “subject” or “individual” can refer to a single subject or individual. In other embodiments, the terms “subject” or “individual” can refer to multiple subjects or individuals.

As used herein, the term “infant” refers to a subject from the age of birth to the age of about 12 months after birth. In some embodiments, the infant is premature, full term, or postmature. As used herein “newborn” refers to an infant from the age of birth to the age of about 2 months after birth.

As used herein, the term “fetus” refers to an unborn offspring, between the embryo stage (the end of about the eighth week after conception, when major structures have formed) until birth.

As used herein, the term “donor” refers to a subject who is a source of a biological material, e.g., blood or blood product. In some embodiments, the donor is a mammal, e.g., a human, a non-human primate, or a horse. In some embodiments, the donor is a plasma and/or serum donor. In some embodiments, the term “donor” can refer to a single donor. In other embodiments, the term “donor” can refer to multiple donors.

As used herein, the terms “at risk for infection” and “at risk for disease” refer to a subject that is predisposed to experiencing a particular infection or disease (e.g., Zika virus infection). This predisposition may be genetic (e.g., a particular genetic tendency to experience the disease, such as heritable disorders), or due to other factors (e.g., immunosuppression, compromised immune system, immunodeficiency, environmental conditions, geography, exposures to detrimental compounds present in the environment, etc.). Thus, it is not intended that the present disclosure be limited to any particular risk (e.g., a subject may be “at risk for disease” simply by being exposed to and interacting with other people).

### Compositions of Zika Virus Neutralizing Antibodies

Certain aspects of the application are directed to compositions (e.g., hyperimmune compositions) comprising antibodies that bind to a Zika virus antigen, e.g., Zika virus neutralizing antibodies.

There are currently no approved treatments or vaccines available for Zika virus infection. Therapeutic efficacy of antibody preparations can be limited due to, for example, low concentrations of specific immunoglobulin directed against the specific target pathogen or impurities. However, the current application overcome these challenges by providing effective compositions (e.g., hyperimmune compositions) of Zika virus neutralizing antibodies for treating, preventing, or reducing the risk of a Zika virus infection, and for other methods disclosed herein. In some embodiments, such compositions, e.g., Zika virus hyperimmune compositions, comprise elevated concentrations of specific immunoglobulins directed to a Zika virus antigen.

In some embodiments, the antibodies of the composition comprise polyclonal antibodies.

In some embodiments, the antibodies of the composition (e.g., polyclonal antibodies) comprise IgG antibodies. In some embodiments, the IgG antibodies of the composition are at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the total protein content of the composition. In some embodiments, the IgG antibodies of the composition are about 50% to 100%, about 60% to 100%, about 70% to 100%, about 80% to 100%, about 90% to 100%, about 95% to 100%, about 50% to about 95%, about 60% to about 95%, about 70% to about 95%, about 80% to about 95%, about 85% to about 95%, about 90% to about 95%, about 50% to about 90%, about 60% to about 90%, about 70% to about 90%, about 80% to about 90%, or about 85% to about 90% of the total protein content of the composition, or any range or value therein.

In some embodiments, the % IgG content can be measured, for example, by chromatography (e.g., size exclusion chromatography) or gel electrophoresis (e.g., SDS-PAGE or agarose gel electrophoresis).

In some embodiments, the antibodies of the composition (e.g., the hyperimmune composition) are from pooled plasma and/or serum samples. In some embodiments, the antibodies are from pooled plasma and/or serum from one or more mammalian donors. In some embodiments, the antibodies are from pooled plasma and/or serum from one or more human donors. In some embodiments, the one or more donors (e.g., mammalian and/or human) were infected with Zika virus prior to pooling plasma and/or serum. In some embodiments, the one or more donors (e.g., mammalian and/or human) were vaccinated with Zika virus vaccine prior to pooling plasma and/or serum. In some embodiments, the one or more donors (e.g., mammalian and/or human) have elevated levels of anti-Zika virus antibodies. In some embodiments, the one or more donors (e.g., mammalian and/or human) have elevated levels of antibodies against a Zika Non-Structural protein 1 (anti-NS1 antibody) and/or a Zika Envelope protein (anti-E-protein antibody).

In some embodiments, the composition (e.g., the hyperimmune composition) comprises antibodies to specific antigens obtained from plasma and/or serum. In some embodiments, the composition (e.g., the hyperimmune composition) is enriched with antibodies specific to one or more particular epitopes of a Zika virus (e.g., anti-NS1 and/or anti-E-protein antibodies). In some embodiments, the composition (e.g., the hyperimmune composition) is prepared from a plasma and/or serum obtained from an individual or pool of individuals with elevated levels of anti-Zika virus antibodies. In some embodiments, the individual or pool of individuals have elevated levels of anti-Zika virus antibodies due to previous exposure to a Zika virus antigen (e.g., an individual or pool of individuals previously infected with a Zika virus). In some embodiments, the individual or pool of individuals have elevated levels of anti-Zika virus antibodies due to intentional stimulation of the immune response (e.g., administration of a Zika vaccine). In some embodiments, the antibodies are immune globulins.

A composition (e.g., a hyperimmune composition) containing Zika virus neutralizing antibodies can be prepared, for example, using the methods described in U.S. Provisional Appl. No. 62/663,972; U.S. Appl. Pub. No. 2017/0336412; U.S. Pat. Nos. 9,107,906; 9,714,283; 9,815,886; Canadian Patent No. 1201063; Int'l Pub. No. WO 98/44948; Int'l Pub. No. WO 2005/069717; Sinclair et al., Biologicals, 36:256-262, 2008; Soluk et al., Am J Therap, 15:435-443, 2008; Schleis, Pharmacother, 25(11 Pt 2):73S-77S, 2005; DiLeo et al., J Chromatog B, 1068-1069:136-148, 2017; Wasserman et al., Exp Rev Clin Immunol, 13(12):1107-1119, 2017; Gerber et al., BioDrugs, 30:441-451, 2016; Williams et al., Chapter 16: Synthetic Ligand Affinity Chromatography Purification, Methods in Molecular Biology, 1129:181-195, 2014; Ma et al., Biologicals, 52:37-43, 2018; Friesen et al., Vox Sanguinis, 48(4):201-212, 1985; and Chapter 14, Price et al., Production of Plasma Proteins for Therapeutic Use by Neil Goss; which are incorporated by reference herein in their entireties.

In some embodiments, a composition comprising Zika virus neutralizing antibodies (e.g., polyclonal antibodies) is a hyperimmune composition.

In some embodiments, the Zika hyperimmune composition neutralizes other flaviviruses (e.g., Dengue).

In some embodiments, the hyperimmune composition is derived from one or more individuals who have been positively diagnosed as being Zika probable (i.e., previously having or likely previously having a Zika virus infection), e.g., using the assays and/or methods disclosed herein or known in the art. In some embodiments, the hyperimmune composition is derived from one or more individuals who have been positively identified to have elevated levels of anti-Zika antibodies, e.g., using the assays and/or methods disclosed herein or known in the art. In other embodiments, the hyperimmune composition is derived from one or more individuals that have been hyper-immunized with one or more Zika virus antigens (e.g., the E-protein, the NS1 protein, and/or whole inactivated or attenuated Zika virus). In some embodiments, at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9%, or at least 10% of the IgG circulating in the Zika virus exposed and/or hyperimmunized individual or individuals are Zika virus specific.

In some embodiments, a hyperimmune composition can be prepared by a method that comprises (a) identifying one or more suitable donors according to the methods disclosed herein, and (b) processing a plasma or serum from the one or more suitable donors to provide the Zika virus hyperimmune composition. In some embodiments, the method further comprises purifying antibodies from the processed plasma or serum. In some embodiments, the method further comprises purifying a Zika virus-specific antibody (including antigen-binding fragments thereof) from the processed plasma or serum. In some embodiments, the purified antibody is an IgG antibody.

In some embodiments, the hyperimmune composition can be prepared, for example, using antibodies from a donor or donors (e.g., a plasma or serum donor or pool of donors) suitable for use in preparing a Zika virus-specific hyperimmune composition. Such a donor or donors can be identified, for example, by a method comprising determining a level of an antibody against a Zika Non-Structural protein 1 (anti-NS1 antibody) and a level of an antibody against a Zika Envelope protein (anti-E-protein antibody) in a biological sample (e.g., a plasma or serum sample or pooled plasma or serum samples) from one or more potential donors; wherein the potential donor is a donor suitable for use in preparing a Zika virus-specific hyperimmune composition if (i) both the anti-Zika-NS1 antibody and the anti-Zika-E-protein antibody are present in the biological sample; and (ii) the ratio of the level of the anti-Zika-NS1 antibody to the level of the anti-Zika-E-protein antibody is greater than a borderline ratio.

Upon identification of one or more donors meeting (i) the ratio criteria or (ii) both the ratio and 20% of the level of the anti-NS1 antibody in a positive control sample criteria, plasma and/or serum from the one or more donors can be collected for preparing the Zika virus hyperimmune composition. In some embodiments, the method for identifying a donor or donors further comprises preparing immunoglobulin from the plasma and/or serum collected from the one or more donors. In some embodiments, the method for identifying a donor or donors further comprises pooling (e.g., from the same or different donors), the collected plasma, collected serum, or prepared immunoglobulin for preparing the Zika virus hyperimmune composition. In some embodiments, the method for identifying a donor or donors further comprises processing the pooled plasma, serum, or immunoglobulin for preparing the Zika virus hyperimmune composition (e.g., IgG purification, viral inactivation and/or removal, microbial inactivation and/or removal, or combinations thereof).

In other embodiments, a donor or donors (e.g., a plasma or serum donor or pool of donors) suitable for use in preparing a hyperimmune composition of the invention can be identified by a method comprising detecting and measuring a particular subset of antibodies (i.e., antibodies that bind to the Zika NS1 and Envelope proteins) from a biological sample of a subject or subjects which can serve as an indicator of a previous Zika virus infection. Such a method can identify, for example, whether a flavivirus-positive biological sample is from a subject or subjects previously infected with a Zika virus or from a subject or subjects previously infected with a non-Zika flavivirus.

In some embodiments, the hyperimmune composition comprises anti-Zika virus neutralizing antibodies which are processed by filtration (e.g., nanofiltration), a separation process (e.g., a two stage separation process), fractionation, chromatography (e.g., ion exchange chromatography, anion exchange chromatography, affinity chromatography, or ligand affinity chromatography), heat treatment, pasteurization, precipitation, removal of procoagulant factors (e.g., Factor XI/Xia), or any combination thereof.

In some embodiments, a composition (e.g., a hyperimmune composition) comprising Zika virus neutralizing antibodies (e.g., polyclonal antibodies) can be prepared, for example, by subjecting a plasma fraction in the form of a dilute aqueous solution containing immunoglobulin to a two stage separation process using two different anionic exchange resins.

In some embodiments, a composition (e.g., a hyperimmune composition) comprising Zika virus neutralizing antibodies (e.g., polyclonal antibodies) can also be prepared, for example, by a manufacturing process incorporating a filtration or nanofiltration step. In some embodiments, the nanofiltration comprises a filter having a pore size of 35 nm or less or 20 nm or less.

In some embodiments, a composition (e.g., a hyperimmune composition) comprising Zika virus neutralizing antibodies (e.g., polyclonal antibodies) can also be prepared, for example, by a manufacturing process incorporating blood plasma fractionation (e.g., Cohn fractionation).

In some embodiments, a composition (e.g., a hyperimmune composition) comprising Zika virus neutralizing antibodies (e.g., polyclonal antibodies) can also be prepared, for example, using anion-exchange chromatography and/or solvent/detergent steps.

In some embodiments, a composition (e.g., a hyperimmune composition) comprising Zika virus neutralizing antibodies (e.g., polyclonal antibodies) contains purified antibodies. Antibody purification can be done by methods known in the art (e.g., chromatography, affinity chromatography, ion exchange chromatography, anion exchange chromatography, ligand affinity chromatography, or other methods described herein).

In some embodiments, a composition (e.g., a hyperimmune composition) comprising Zika virus neutralizing antibodies (e.g., polyclonal antibodies) has been treated to inactivate viruses and/or microbes. Viral and microbial inactivation and/or removal can be done by methods known in the art (e.g., heat treatment, pasteurization, solvent/detergent, low pH solutions, precipitation, chromatography, affinity chromatography, ion exchange chromatography, anion exchange chromatography, filtration, nanofiltration, chemical alteration of viral RNA, treatment with methylene blue, psoralens, riboflavin, and/or caprylate, or other methods described herein and known in the art).

In some embodiments, a composition (e.g., a hyperimmune composition) comprising Zika virus neutralizing antibodies (e.g., polyclonal antibodies) can comprise blood and/or blood product (e.g., plasma, serum, immunoglobulins, or any combination thereof).

In some embodiments, a composition (e.g., a hyperimmune composition) comprising Zika neutralizing antibodies has a percent neutralization of at least 0.25%, at least 0.50%, at least 0.75%, or at least 1%.

### Methods of Use

Certain aspects of the application are directed to methods of using any of the compositions containing Zika virus antibodies disclosed herein (e.g., Zika virus neutralizing antibody composition, e.g., a Zika hyperimmune composition).

There are currently no approved treatments or vaccines available for Zika virus infection. The methods of the current application provide effective compositions of Zika virus neutralizing antibodies (e.g., a Zika hyperimmune composition) for treating, preventing or reducing the risk of a Zika virus infection, for reducing the symptoms associated with a Zika virus infection, for protecting an embryo, fetus and/or infant from a Zika virus infection, and/or for the other methods and uses disclosed herein.

In some embodiments, the present disclosure relates to a method for treating, preventing or reducing the risk of a Zika virus infection. In some embodiments, the Zika virus infection is associated with birth defects or congenital Zika syndrome. In some embodiments, the congenital Zika syndrome includes one or more of the following features: severe microcephaly in which the skull has partially collapsed; decreased brain tissue with a specific pattern of brain damage, including subcortical calcifications; damage to the back of the eye, including macular scarring and focal pigmentary retinal mottling; congenital contractures, such as clubfoot or arthrogryposis; and hypertonia restricting body movement soon after birth. In some embodiments, the congenital Zika syndrome is associated with one or more of the following abnormalities: brain atrophy and asymmetry, abnormally formed or absent brain structures, hydrocephalus, and neuronal migration disorders.

In some embodiments, the method comprises administering to a subject in need thereof an effective amount of a composition comprising Zika virus neutralizing antibodies (e.g., a Zika hyperimmune composition). In some embodiments, the neutralizing antibodies are polyclonal antibodies. In some embodiments, the antibodies (e.g., polyclonal antibodies) are from pooled plasma and/or serum. In some embodiments, the antibodies (e.g., polyclonal antibodies) are from one or more mammalian donors. In some embodiments, the antibodies (e.g., polyclonal antibodies) are from one or more human donors.

In some embodiments, the present disclosure relates to a method for reducing viral load of a Zika virus in a sample of a subject (e.g., bodily fluid, tissue, or cell). In some embodiments, the method comprises administering to the subject an effective amount of a composition comprising Zika virus neutralizing antibodies (e.g., a Zika hyperimmune composition). In some embodiments, the neutralizing antibodies are polyclonal antibodies. In some embodiments, the antibodies (e.g., polyclonal antibodies) are from pooled plasma and/or serum. In some embodiments, the antibodies (e.g., polyclonal antibodies) are from one or more mammalian donors. In some embodiments, the antibodies (e.g., polyclonal antibodies) are from one or more human donors.

In some embodiments, the present disclosure relates to a method of eliciting an immune response against a Zika virus. In some embodiments, the method comprises administering an effective amount of a composition comprising Zika virus neutralizing antibodies (e.g., a Zika hyperimmune composition) to a subject in need thereof. In some embodiments, the neutralizing antibodies are polyclonal antibodies. In some embodiments, the antibodies (e.g., polyclonal antibodies) are from pooled plasma and/or serum. In some embodiments, the antibodies (e.g., polyclonal antibodies) are from one or more mammalian donors. In some embodiments, the antibodies (e.g., polyclonal antibodies) are from one or more human donors.

In some embodiments, the present disclosure relates to a method of passive immunization against a Zika virus. In some embodiments, the method comprises administering an effective amount of a composition comprising Zika virus neutralizing antibodies (e.g., a Zika hyperimmune composition) to a subject in need thereof. In some embodiments, the neutralizing antibodies are polyclonal antibodies. In some embodiments, the antibodies (e.g., polyclonal antibodies) are from pooled plasma and/or serum. In some embodiments, the antibodies (e.g., polyclonal antibodies) are from one or more mammalian donors. In some embodiments, the antibodies (e.g., polyclonal antibodies) are from one or more human donors.

In some embodiments, the present disclosure relates to a method of preventing or reducing the risk of transmission of a Zika virus infection from a subject to an embryo, fetus, or infant. In some embodiments, the method comprises administering to the subject an effective amount of a composition comprising Zika virus neutralizing antibodies (e.g., a Zika hyperimmune composition). In some embodiments, the neutralizing antibodies are polyclonal antibodies. In some embodiments, the antibodies (e.g., polyclonal antibodies) are from pooled plasma and/or serum. In some embodiments, the antibodies (e.g., polyclonal antibodies) are from one or more mammalian donors. In some embodiments, the antibodies (e.g., polyclonal antibodies) are from one or more human donors. In some embodiments, the method prevents or reduces the vertical transmission of a Zika virus infection from a pregnant subject to an embryo, fetus or infant of the subject.

In some embodiments, the present disclosure relates to a method of preventing or reducing the risk of transmission of a Zika virus from a subject. In some embodiments, the transmission is from a male subject to a female subject. In some embodiments, the transmission is from a female subject to a male subject. In some embodiments, the transmission is vertical transmission. In some embodiments, the transmission is from a female subject to an embryo, a fetus, or an infant.

In some embodiments, the method comprises administering to the subject an effective amount of a composition comprising Zika virus neutralizing antibodies (e.g., a Zika hyperimmune composition). In some embodiments, the neutralizing antibodies are polyclonal antibodies. In some embodiments, the antibodies (e.g., polyclonal antibodies) are from pooled plasma and/or serum. In some embodiments, the antibodies (e.g., polyclonal antibodies) are from one or more mammalian donors. In some embodiments, the antibodies (e.g., polyclonal antibodies) are from one or more human donors. In some embodiments, the subject is trying to become pregnant.

In some embodiments, the present disclosure relates to a method of treating, preventing, or reducing the risk of a Zika virus infection in an embryo, a fetus, or an infant. In some embodiments, the method comprises administering an effective amount of a composition comprising Zika virus neutralizing antibodies (e.g., a Zika hyperimmune composition) to a subject pregnant with the embryo or the fetus. In some embodiments, the subject is pregnant and has a Zika virus infection before and/or during the birth of the infant. In some embodiments, the neutralizing antibodies are polyclonal antibodies. In some embodiments, the antibodies (e.g., polyclonal antibodies) are from pooled plasma and/or serum. In some embodiments, the antibodies (e.g., polyclonal antibodies) are from one or more mammalian donors. In some embodiments, the antibodies (e.g., polyclonal antibodies) are from one or more human donors. In some embodiments, the present disclosure relates to a method of preventing or reducing the severity or risk of congenital Zika syndrome. In some embodiments, the present disclosure relates to a method of preventing or reducing the severity or risk of microcephaly (e.g., partial collapse of the skull); brain damage (e.g., subcortical calcifications, brain atrophy and asymmetry, abnormally formed or absent brain structures, hydrocephalus, and neuronal migration disorders); damage to the eye (e.g., macular scarring and focal pigmentary retinal mottling); congenital contractures (e.g., clubfoot or arthrogryposis); hypertonia; and any combination thereof. In some embodiments, the present disclosure relates to a method of preventing or reducing the severity or risk of microcephaly in a fetus. In some embodiments, the method comprises administering to a pregnant subject carrying the fetus an effective amount of a composition comprising Zika virus neutralizing antibodies (e.g., a Zika hyperimmune composition). In some embodiments, the neutralizing antibodies are polyclonal antibodies. In some embodiments, the antibodies (e.g., polyclonal antibodies) are from pooled plasma and/or serum. In some embodiments, the antibodies (e.g., olyclonal antibodies) are from one or more mammalian donors. In some embodiments, the antibodies (e.g., polyclonal antibodies) are from one or more human donors.

In some embodiments of any of the methods disclosed herein, the subject, embryo, fetus and/or infant is a mammal. In some embodiments, the subject, embryo, fetus and/or infant is human. In some embodiments, the subject and fetus are human. In some embodiments, the subject, embryo, fetus and/or infant is male. In some embodiments, the subject, embryo, fetus, and/or infant is female. In some embodiments, the subject is pregnant, suspected of being pregnant, or trying to become pregnant. In some embodiments, the subject is in the first trimester, second trimester, or third trimester of pregnancy. In some embodiments, the subject is in the late stage of the first trimester or early stage of the second trimester of pregnancy. In some embodiments, the subject is pregnant and transmission of Zika virus from the pregnant subject to the embryo or the fetus is prevented, reduced or eliminated. In some embodiments, the subject is pregnant and a Zika virus antigen-specific immune response is produced in the embryo and/or fetus.

In some embodiments of any of the methods disclosed herein, the subject has been bitten by a mosquito suspected of harboring the Zika virus, lives in an area that had or has a Zika virus outbreak, is visiting or has visited an area that had or has a Zika virus outbreak, is immunocompromised, is suspected of having been exposed to a person harboring the Zika virus, has come into physical contact or close physical proximity with an infected individual, is a hospital employee, and/or lives in or is visiting a country or region known to have mosquitoes harboring the Zika virus. In some embodiments, the subject has been diagnosed with having or is suspected of having African lineage Zika virus strain, Asian lineage Zika virus strain, Brazil lineage virus strain, or Puerto Rico lineage virus strain. In some embodiments, the subject has been diagnosed with having or is suspected of having Zika virus strain MR 766, FLR, Brazil-ZKV2015, or PRVABC59.

In some embodiments of any of the methods disclosed herein, an effective amount of a composition comprising Zika virus neutralizing antibodies (e.g., a Zika hyperimmune composition) is used. In some embodiments, the effective amount is sufficient to provide a Zika virus antigen-specific immune response in the subject, embryo, fetus and/or infant. In some embodiments, the effective amount is sufficient to neutralize the Zika virus in the subject, embryo, fetus and/or infant.

In some embodiments of any of the methods disclosed herein, the composition (e.g., a Zika hyperimmune composition) is administered as at least one dose of about 50 mg/kg to about 400 mg/kg, or any range or values therein. In some embodiments, the composition is administered as at least one dose of about 50 mg/kg to about 350 mg/kg, about 50 mg/kg to about 300 mg/kg, about 50 mg/kg to about 250 mg/kg, about 50 mg/kg to about 200 mg/kg, about 50 mg/kg to about 150 mg/kg, about 50 mg/kg to about 100 mg/kg, about 100 mg/kg to about 400 mg/kg, about 100 mg/kg to about 350 mg/kg, about 100 mg/kg to about 300 mg/kg, about 100 mg/kg to about 250 mg/kg, about 100 mg/kg to about 200 mg/kg, about 100 mg/kg to about 150 mg/kg, about 150 mg/kg to about 400 mg/kg, about 150 mg/kg to about 350 mg/kg, about 150 mg/kg to about 300 mg/kg, about 150 mg/kg to about 250 mg/kg, about 150 mg/kg to about 200 mg/kg, about 200 mg/kg to about 400 mg/kg, about 200 mg/kg to about 350 mg/kg, about 200 mg/kg to about 300 mg/kg, about 200 mg/kg to about 250 mg/kg, about 250 mg/kg to about 400 mg/kg, about 250 mg/kg to about 350 mg/kg, about 300 mg/kg to about 400 mg/kg, about 300 mg/kg to about 350 mg/kg, or about 350 mg/kg to about 400 mg/kg. In some embodiments, the composition is administered as at least one dose of about 50 mg/kg, about 100 mg/kg, about 150 mg/kg, about 200 mg/kg, about 250 mg/kg, about 300 mg/kg, about 350 mg/kg, about 400 mg/kg, about 450 mg/kg, or about 500 mg/kg.

In some embodiments of any of the methods disclosed herein, the composition is administered by parenteral administration. In some embodiments, the composition is administered by intravenous, intraperitoneal, intramuscular, subcutaneous, intrauterinal, or spinal administration, for example, by injection or infusion. In some embodiments, the composition by non-parenteral administration. In some embodiments, the composition is administered by topical, epidermal or mucosal administration, for example, intranasally, orally, vaginally, rectally, sublingually or topically.

In some embodiments of any of the methods disclosed herein, administering can be performed once, twice, a plurality of times, and/or over one or more extended periods.

In some embodiments of any of the methods disclosed herein, the method further comprises passive immunization of the fetus.

In some embodiments of any of the methods disclosed herein, the risk of miscarriage and/or stillbirth is reduced.

In some embodiments of any of the methods disclosed herein, the method comprises administration of the composition comprising Zika virus neutralizing antibodies (e.g., a Zika hyperimmune composition) before the subject is infected with the Zika virus, after the subject has been infected with the Zika virus, or after the subject has been exposed to or is suspected of having been exposed to the Zika virus and before the Zika virus infection can be detected.

In some embodiments of any of the methods disclosed herein, the administration treats, prevents or reduces the risk of one or more symptoms associated with Zika virus infection or treats, prevents or reduces the risk of a disease or disorder associated with a Zika virus infection. In some embodiments, the one or more symptoms associated with the Zika virus infection comprise a fever, rash, headache, joint pain, conjunctivitis, muscle pain, lethargy, myalgia, and arthralgia. In some embodiments, a disease or disorders associated with a Zika virus infection includes neurotropic Guillain-Barre syndrome and congenital microcephaly.

In some embodiments of any of the methods disclosed herein, wherein duration of Zika viremia in the subject, fetus, embryo and/or infant is shortened.

In some embodiments of any of the methods disclosed herein, the Zika viral load in the blood and/or a tissue of the subject, fetus, embryo and/or infant is prevented or decreased. In some embodiments, the Zika viral load in the blood and/or tissue is decreased by at least 25%, at least 50%, at least 75%, at least 80%, at least 85%, at least 90%, at least 95%, at least 99%, or 100%. In some embodiments, the Zika viral load is decreased by about 25% to about 100%, or any range or values therein, for example, about 50% to about 100%, about 75% to about 100%, about 80% to about 100%, about 90% to about 100%, about 95% to about 100%, about 25% to about 95%, about 50% to about 95%, about 75% to about 95%, about 80% to about 95%, about 85% to about 95%, about 90% to about 95%, about 25% to about 90%, about 50% to about 90%, about 75% to about 90%, or about 80% to about 90%.

In some embodiments, the tissue is brain, dura mater, spinal cord, sciatic nerve, cochlea, cerebrum, cerebellum, aqueous humor, optic nerve, sclera, cornea, retina, pericardium, heart, aorta, lung, seminal vesicle, prostate/uterus, testis, ovary, articular cartilage, adipose tissue-omentus, epidermis/dermis of abdomen, muscle-quadriceps, bone marrow, tonsil, spleen, thymus, lymph nodes, gastric contents, esophagus, stomach, duodenum, jejunum, ileum, cecum, colon, bile aspirate, liver, meconium, tongue, urinary bladder, kidney, urine, thyroid, adrenal gland, pituitary, pancreas, fetal blood, placental disk, uterus, decidua, amniotic/chorionic membrane, amniotic fluid, umbilical cord, cord blood, or any combination thereof.

In some embodiments of any of the methods disclosed herein, the composition is administered with one or more additional therapeutic agents.

In some embodiments of any of the methods disclosed herein, the composition is administered before or after a Zika virus infection, e.g., within 1, 2, 4, 6, 12, 24, 36, 48, or 60 hours before or after infection or after detection of symptoms, or even at a later time.

In some embodiments of any of the methods disclosed herein, the sample of the subject is a biological sample. In some embodiments, the sample is a body fluid sample such as whole blood, serum, plasma, urine, saliva, seminal fluid, cerebrospinal fluid, or a combination thereof.

In some embodiments, the Zika virus neutralizing antibody composition, e.g., a Zika hyperimmune composition, disclosed herein can also be against other flaviviruses (e.g., Dengue). In some embodiments, the Zika hyperimmune composition neutralizes other flaviviruses (e.g., Dengue). Some aspect of the application are directed to a method for treating, preventing or reducing the risk of a flavivirus virus infection comprising administering the Zika virus neutralizing antibody composition, e.g., a Zika hyperimmune composition, of the disclosure.

The following examples are merely illustrative and should not be construed as limiting the scope of this disclosure in any way as many variations and equivalents will become apparent to those skilled in the art upon reading the present disclosure.

Animal models that can be used experimentally for evaluating Zika virus treatments include, but are not limited to, non-human primates, mice and guinea pigs.

The contents of all references, GenBank entries, patents and published patent applications cited throughout this application are expressly incorporated herein by reference.

### EXAMPLES

### Example 1: Efficacy of ZIKA-IG Polyclonal Antibodies in a Lethal Model of Zika Virus Infection in Ifnar1−/− Mice

To evaluate the efficacy of ZIKV-IG in mice challenged with a lethal dose of ZIKV, the following study was performed.

### Study

Forty-eight interferon-alpha/beta receptor alpha chain knock-out (Ifnar1−/−) mice between the ages of 5-7 weeks were divided into five treatment groups (B through E) and one control group (A). Mice were infected intravenously (IV) with Zika virus (ZIKV; Asian lineage strain FSS13025) at a dose of 1.0×10³ focus forming units (FFU)/mouse. In addition, mice were administered with a single IV dose of ZIKV-IG polyclonal antibodies either (i) 1 hour before infection at two different dose levels (100 mg/kg or 50 mg/kg) or (ii) 24 hours post-infection at three different dose levels (400 mg/kg, 100 mg/kg or 50 mg/kg), or with PBS (vehicle/negative control).

Following infection, animals were monitored daily for body weight change, clinical signs, and survival for up to 21 days. More specifically, all animals were observed for outward signs of disease once daily and scored using a numerical scoring system, as described in Tang et al., Cell Rep., 17(12):3091, 3098, 2016. Mice were weighed on Day 0 pre-infection to establish a baseline, and body weights were recorded every day for a period of 21 days post-infection.

### Results

Survival

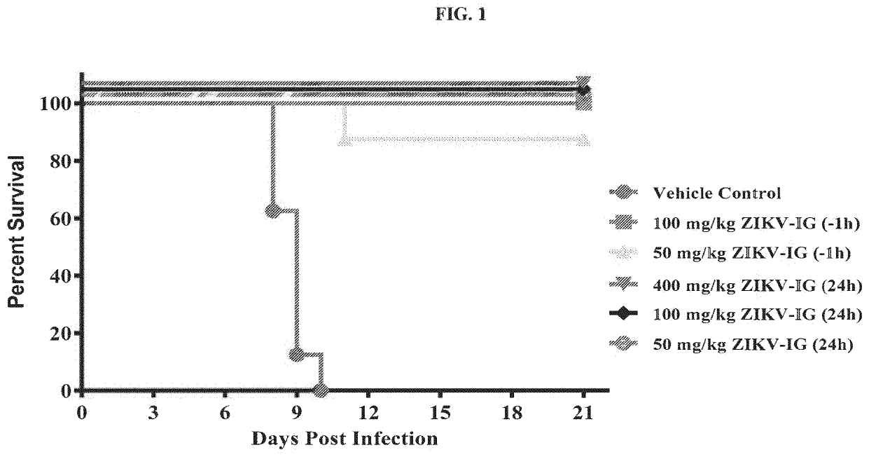

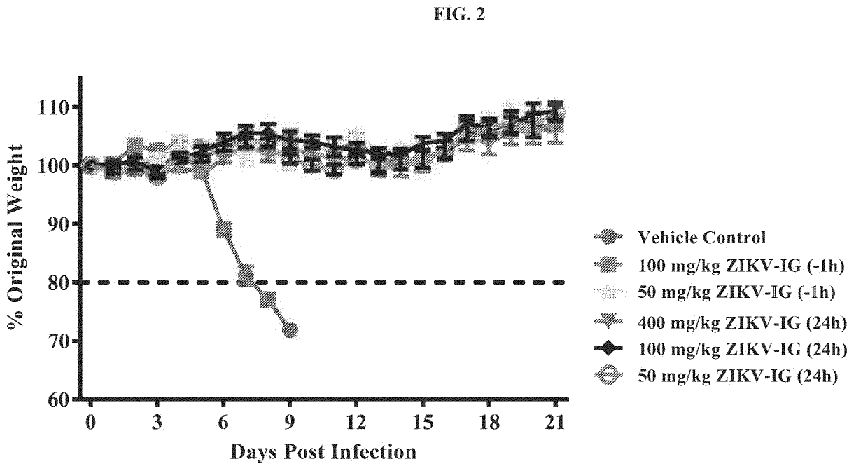

As shown in FIG. 1 and Table 1, all vehicle treated mice (Group A) succumbed to ZIKV infection with 100% mortality by day 10. However, all mice pre-treated with ZIKV-IG at 100 mg/kg and 50 mg/kg (Groups B and C, respectively) survived, except one mouse in Group C (1/8) which died on day 11 with hydrocephaly. The death of this animal did not appear to be related to ZIKV-IG.

TABLE 1
Comparison of treatment and control groups survival rates.

| | Number of | Treatment | | | |
|---|---|---|---|---|---|
| Group | mice treated | (200 μL/dose) | Survival | | P Value* |
| A | 8 | 1x PBS | 0% | (0/8) | Not |
| | | | | | applicable |
| B | 8 | 100 mg/kg (−1 hr) | 100% | (8/8) | <0.0001 |
| C | 8 | 50 mg/kg (−1 hr) | 87.5% | (7/8) | <0.0001 |
| D | 8 | 400 mg/kg (+24 hrs) | 100% | (8/8) | <0.0001 |
| E | 8 | 100 mg/kg (+24 hrs) | 100% | (8/8) | <0.0001 |
| F | 8 | 50 mg/kg (+24 hrs) | 100% | (8/8) | <0.0001 |

*Kaplan-Meier survival curves are analyzed using the Mantel-Cox log-rank test followed by the Gehan-Breslow-Wilcoxon test on Prism software (GraphPad). P < 0.05 indicates statistical significance between vehicle-treated group and indicated comparison group.

All mice treated with ZIKV-IG at 400 mg/kg, 100 mg/kg or 50 mg/kg one day post-exposure (Groups D, E and F, respectively), survived (100%). Overall, all ZIKV-IG treated groups had statistically significant survival compared to control (p<0.0001).

Body Weight

As shown in FIG. 2 and Table 2, mice treated with vehicle (PBS) lost significantly more weight than ZIKV-IG treated mice starting from day 6 post-infection. All control mice lost weight and succumbed to ZIKV infection while ZIKV-IG treated groups gained weight (above baseline) by the end of day 21 post-infection. By comparison, the body weight change (gain) in all treated groups was significantly higher compared to the vehicle/control group (p<0.001) on days 6-8 post-infection.

TABLE 2
Statistical analysis of body weight changes between
treatment and control groups on various study days

| Study | 100 mg/kg | 50 mg/kg | 400 mg/kg | 100 mg/kg | 50 mg/kg |
|---|---|---|---|---|---|
| day | (−1 hr) | (−1 hr) | (+24 hrs) | (+24 hrs) | (+24 hrs) |
| 1 | 0.89679 | 0.112379 | 0.057139 | 0.916073 | 0.147407 |
| 2 | 0.103048 | 0.998171 | 0.171050 | 0.892017 | 0.435119 |
| 3 | 0.020686* | 0.207734 | 0.181691 | 0.449217 | 0.065866 |
| 4 | 0.199439 | 0.174502 | 0.092795 | 0.540157 | 0.986716 |
| 5 | 0.021711* | 0.011902* | 0.655481 | 0.050134 | 0.141316 |
| 6 | <0.001* | <0.001* | <0.001* | <0.001* | <0.001* |
| 7 | <0.001* | <0.001* | <0.001* | <0.001* | <0.001* |
| 8 | <0.001* | <0.001* | <0.001* | <0.001* | <0.001* |

*Results were analyzed by Student t-test and were considered statistically significant.

Clinical Observations

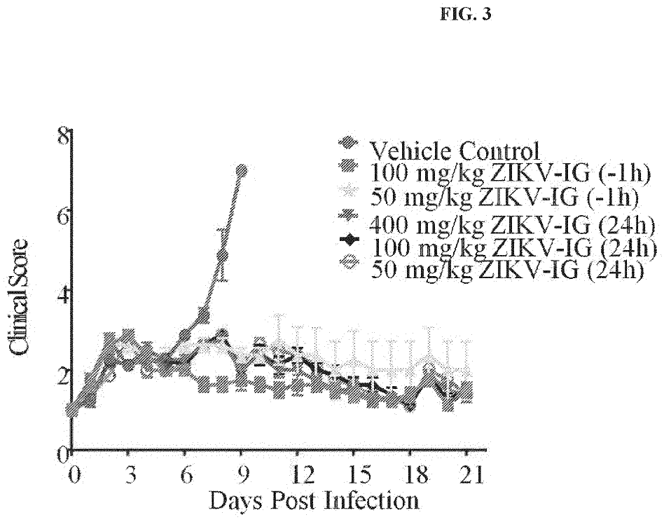

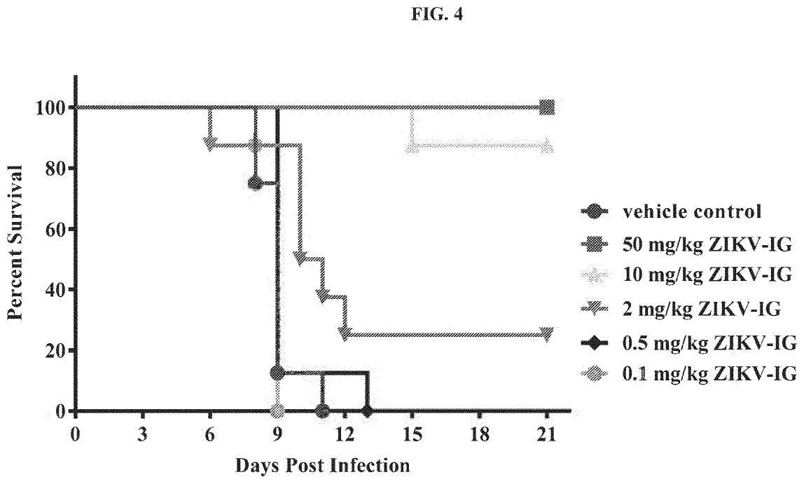

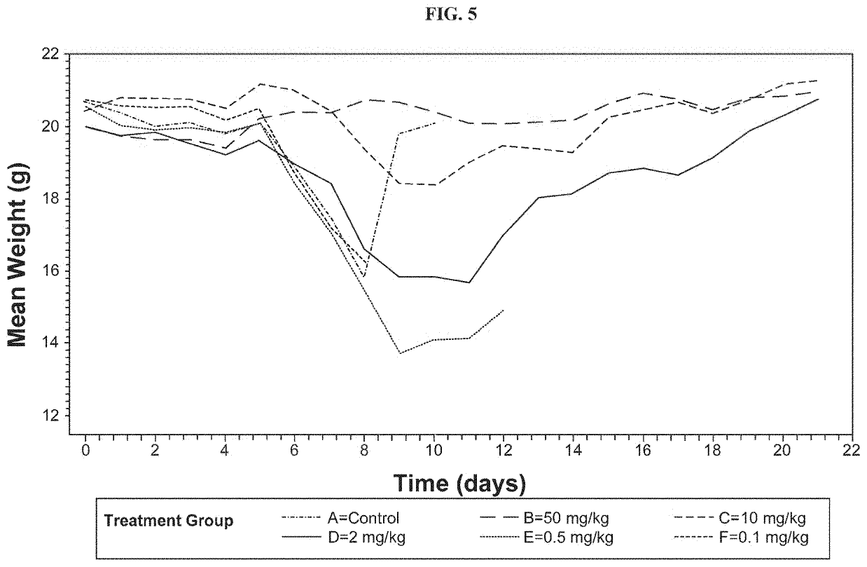

As shown in FIG. 3, the clinical score of control treated mice progressively increased until all mice succumbed to lethal ZIKV disease. In contrast, the clinical score of all ZIKV-IG treated mice reached 3 and declined prior to a full recovery, with the exception of one mouse from the group treated pre-exposure to ZIKV at 50 mg/kg ZIKV-IG that developed hydrocephaly.

### Conclusion

Based on the results obtained, ZIKV-IG administration resulted in significant survival benefit against ZIKV infection when given either pre- or post-ZIKV infection. All control mice succumbed to lethal ZIKV infection and all but one ZIKV-IG treated mice (both pre- and post-exposure) survived. In addition, treated mice completely recovered as demonstrated by the reduced clinical score and body weight gain by the end of the study.

### Example 2: Dose-Ranging Study of ZIKA-IG Polyclonal Antibodies in a Lethal Model of Zika Virus Infection in Ifnar1−/− Mice

The following study analyzed the dose-ranging effect of ZIKV-IG on the overall survival, body weight change, median time to death (MTD) and severity of clinical disease between ZIKV-IG treated and control groups. In addition, the potential of ZIKV-IG to enhance ZIKV infection when administered at lower dose levels in Ifnar1−/− mice was also analyzed.

### Study

Mice were randomized into five treatment and one control group (n=8/group). All mice were challenged via intravenous (IV) route with Zika virus (ZIKV; FSS13025) at a dose of 1.0×10³ FFU/mouse. Following challenge, mice were treated by IV with a vehicle control or one of the five doses of ZIKV-IG at 24 hours after infection. The doses of ZIKV-IG tested were 50 mg/kg, 10 mg/kg, 2 mg/kg, 0.5 mg/kg, and 0.1 mg/kg (based on total protein concentration). Following infection, animals were observed for survival, outward signs of the disease (daily for 21 days), and scored using the same numerical scoring system described in Example 1. Mice were weighed on Day −1 and daily thereafter for body weight assessment for up to 21 days post-infection.

### Results

The results of the study demonstrated a significant dose-related enhancement of survival with ZIKV-IG treatment when compared to control. Additionally, there were no differences between the median time to death (MTD), weight loss or clinical severity of the disease in groups treated with lower dose levels (0.5 and 0.1 mg/kg) compared to the control.

Survival

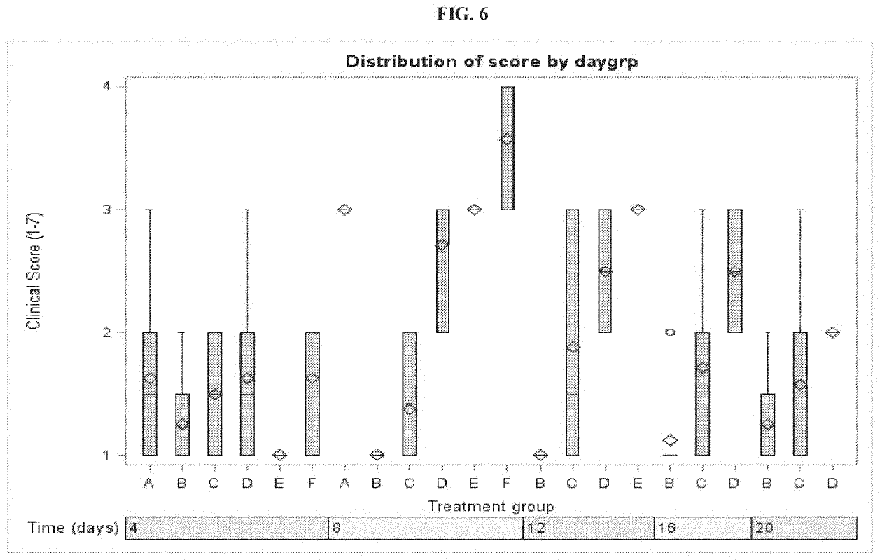

As shown in FIG. 4, none of the animals treated with the vehicle control survived following lethal ZIKV infection. However, 17 of the 40 animals (42.5% treated with ZIKV-IG survived to the end of the study. Survival curves, calculated as percent survival versus days post-infection for these animals are shown in FIG. 4. The groups treated with single doses of ZIKV-IG at 50 mg/kg (B), 10 mg/kg (C), and 2 mg/kg (D) showed survival rates of 100%, 87.5%, and 25% respectively, while the vehicle control group A and ZIKV-IG groups treated at 0.5 mg/kg (E) and 0.1 mg/kg (F) had 0% survival.

Differences in survival rates were statistically significant for the control group versus the 50 mg/kg treated group (Adjusted p=0.0005) and the control group versus the 10 mg/kg treated group (Adjusted p=0.0042). Based on these results, the survival rate in ZIKV-IG treated animals (50 and 10 mg/kg) is higher than the survival rate of controls. In addition, as shown in Table 3, the observed survival is dose-related and increased with increasing dose.

TABLE 3
Statistical analysis of survival rates at 21 days post challenge by treatment group.

| | ZIKV-IG | | | 95% Exact | Fisher's | Bonferroni |
|---|---|---|---|---|---|---|
| | Treatment | Survival | | Binomial | Exact Test | Adjusted |
| | Dose | Rate | | Confidence | p-value vs Group A | p-value vs Group A |
| Group | (mg/kg) | (%) | | Interval | (* = significant) | (* = significant) |
| A | 0 (PBS) | 0/8 | (0) | (0.00, 0.37) | — | — |
| B | 50 | 8/8 | (100) | (0.63, 1.00) | 0.00016* | 0.0005* |
| C | 10 | 7/8 | (88) | (0.47, 1.00) | 0.00140* | 0.0042* |
| D | 2 | 2/8 | (25) | (0.03, 0.65) | 0.467 | 1.0000 |
| E | 0.5 | 0/8 | (0) | (0.00, 0.37) | — | — |
| F | 0.1 | 0/8 | (0) | (0.00, 0.37) | — | — |

Median Time to Death

As shown in Table 4, the median time to death (MTD) at corresponding 95% confidence intervals were calculated for each treatment group using Kaplan-Meier estimates. Statistically significant differences were found in the survival time distributions for control versus 50 mg/kg and control versus 10 mg/kg using Log-rank tests with Bonferroni correction (p=0.0001, p=0.0008). The MTD was between 9-10.5 days for PBS and lower dose groups (2 mg/kg, 0.5 mg/kg, and 0.1 mg/kg) and there was no significant difference in the MTD between these groups.

TABLE 4
Analysis of median time to death of treatment groups.

| | ZIKV-IG | | | | Bonferroni |
|---|---|---|---|---|---|
| | Treatment | Median Time | 95% | Log rank Test | Adjusted |
| | Dose | to Death | Confidence | p-value | p-value |
| Group | (mg/kg) | (days) | Interval | (* = significant) | (* = significant) |
| A | 0 (PBS) | 9.00 | (8.00, 9.00) | — | — |
| B | 50 | — | (—, —) | <0.0001* | 0.0001* |
| C | 10 | — | (15.0, —) | <0.0001* | 0.0008* |
| D | 2 | 10.50 | (6.00, —) | 0.126 | 1.000 |
| E | 0.5 | 9.00 | (—, —) | 0.675 | 1.000 |
| F | 0.1 | 9.00 | (8.00, 9.00) | 0.919 | 1.000 |

Body Weight Changes

As shown in FIG. 5, the vehicle control (A), 0.5 mg/kg (E), and 0.1 mg/kg (F) treated groups continued to lose weight after infection and succumbed to infection and were euthanized prior today 21. The body weights of animals treated with the highest dose (50 mg/kg) of ZIKV-IG remained high (above baseline) throughout the study. Animals treated with 10 mg/kg (C) and 2 mg/kg (B) exhibited weight loss around days 9-11, followed by weight gains for the surviving animals.

The body weight loss in all groups (≥20% body weight) from baseline at any time in the study were analyzed by Fisher's exact test. Similar to the survival rate results, differences in ≥20% body weight change (loss) rates were statistically significant for control versus 50 mg/kg group (Adjusted p=0.007) and near significant for control versus 10 mg/kg dose group C (Adjusted p=0.05). There was a significant difference between high ZIKV-IG dose groups (1/8 in 10 mg/kg group and 0/8 in 50 mg/kg group experienced a ≥20% loss from baseline) and the control (7/8 from control group lost ≥20% from baseline) group. There was no significant difference between low dose (2 mg/kg, 0.5 mg/kg and 1 mg/kg) groups and the control group (p-value=1), since all animals in these groups experienced a ≥20% body weight change (loss). These results indicate a dose-related treatment effect with ZIKV-IG.

Clinical Health Observations

Clinical scores over time for each treatment group are show in FIG. 6 using boxplots for selected study days. The diamond symbol represents the median, the horizontal line represents the mean, the box represents the interquartile range (25th percentile to 75th percentile), and the bars represent the minimum and maximum values. At day 8, all clinical scores in the vehicle control (A) and 0.5 mg/kg (E) treated groups were 3, but all clinical scores in the 50 mg/kg ZIKV-IG treated group were less than 2, suggesting no disease progression in this group.

Clinical scores at day 8 in the 10 mg/kg were 1-2, 2 mg/kg was 2-3, and 0.1 mg/kg (treated with the lowest dose of ZIKV-IG) was 3-4. By day 12/13, all animals in the vehicle control and 0.1 mg/kg groups were deceased or euthanized. The remaining animals showed a trend toward higher clinical scores for lower ZIKV-IG doses at days 12, 16, and 20.

Clinical severity of the disease was also analyzed using Jonckheere Terpstra exact test. These results are shown in Table 5. The Jonckheere-Terpstra test is a rank-based nonparametric test used to determine a statistically significant trend between an ordinal independent variable (dose) and an ordinal dependent variable (clinical score). All the clinical scores recorded for each animal in each group were used in this analysis, without regard to their timing. The ordinal analysis suggested that the clinical scores were linearly higher (indicating the higher severity of the clinical disease) in animals given lower doses of ZIKV-IG, despite attrition by death or euthanasia. There was a statistically significant difference between the control group versus the 50 mg/kg treatment group (Bonferroni Adjusted p<0.0001) and the control group versus the 10 mg/kg treatment group (Bonferroni Adjusted p=0.0286) with respect to severity of the disease. There was no statistically significant difference between the lower dose groups (2 mg/kg to 0.1 mg/kg) and the control group.

TABLE 5
Ordinal analysis of clinical scores of treatment groups

| | | Exact Jonckheere | |
|---|---|---|---|
| | ZIKV-IG | Terpstra Test | Bonferroni Adjusted |
| | Treatment Dose | p-value vs Group A | p-value vs Group A |
| Group | (mg/kg) | (* = significant) | (* = significant) |
| A | 0 (PBS) | — | — |
| B | 50 | <0.0001* | <0.0001* |
| C | 10 | 0.0057* | 0.0286* |
| D | 2 | 0.134 | 0.672 |
| E | 0.5 | 0.057 | 0.283 |
| F | 0.1 | 0.697 | 1.00 |

### Conclusion

Post-exposure prophylaxis ZIKV-IG was highly effective when administered following a lethal exposure to ZIKV. The groups treated with a single dose of ZIKV-IG at 50 mg/kg, 10 mg/kg, or 2 mg/kg dose levels 24 hours after lethal ZIKV infection resulted in a survival rate of 100%, 87.5%, and 25%, respectively. In comparison, there was 100% mortality observed for the control (PBS) group. Overall, the higher doses of ZIKV-IG demonstrated a statistically significant (p<0.005) survival benefit compared to control.

A Kaplan-Meier estimate of median time to death (MTD) was between 9-10.5 days for control and lower dose treatment groups (2 mg/kg to 0.1 mg/kg). The MTD was statistically significant between the 50 mg/kg treatment group (p=0.0001, Log-rank test with Bonferroni correction) and 10 mg/kg treatment group (p=0.0008) compared to the control group. The MTD between the lower dose groups (2, 0.5 and 0.1 mg/kg) and the control group was not statistically different.

The body weight change analysis indicated that the body weight of animals treated with the highest dose (50 mg/kg) of ZIKV-IG remained high throughout the study. There was a significant difference between higher ZIKV-IG dose groups and rest of the groups (lower dose and control groups) with respect to loss of body weight. There was no significant difference between lower dose (2.0 mg/kg to 0.1 mg/kg) groups and the control group (p-value for groups 2 mg/kg and 0.1 mg/kg was 1.00) since most animals in these groups (except 2/8 in 2 mg/kg group) experienced a ≥20% body weight change.

Clinical scores over time for each treatment group were also analyzed. The clinical scores between days 8-12 are relevant for disease severity analysis, as the majority of the controls display severe disease signs and succumb to infection during this period. At day 8 post-infection, all animals treated with higher dose levels showed minimal clinical scores compared to the lower doses of ZIKV-IG. These results showed that there was a reduced severity of the disease in high dose groups compared to the control and low dose groups. The clinical scores were linearly higher (indicating the higher severity of the clinical disease) in animals given lower doses of ZIKV-IG.

The results showed no significant differences between the control group and lower dose treatment groups (ZIKV-IG at 2 mg/kg to 0.1 mg/kg) in terms of MTD, clinical severity scores and body weight loss kinetics under the testing conditions.

### Example 3: Dose-Ranging Study of ZIKA-IG Polyclonal Antibodies in a Lethal Model of Zika Virus Infection in Ifnar1−/− Mice—Viral Load

A study was designed to analyze the dose-ranging effect of ZIKV-IG on the overall viral load in target tissues between treatment and control groups.

### Study

For this analysis, both quantitative reverse transcriptase-polymerase chain reaction (qRT-PCR) and focus-forming assay (FFA) were utilized. The treatment effect on virus neutralization and prevention of the spread of the virus to the key organs and tissues on day 3 and 7 following infection were assessed. In addition, the potential of ZIKV-IG to enhance ZIKV infection when administered at lower dose levels in terms of viral load in key organs and tissues was evaluated.

Mice were randomized into four treatment groups and one control group (n=12/group). All mice were challenged intravenously with Zika virus (ZIKV) at a dose of 1.0×10³ FFU/mouse. Following challenge, mice were treated intravenously with PBS or one of 4 doses of ZIKV-IG at 24 hours post-infection. The doses of ZIKV-IG tested were 50 mg/kg, 10 mg/kg, 2 mg/kg, and 0.5 mg/kg. Following infection, 6 mice from each group (3 females and 3 males/per group) were sacrificed on each of day 3 and day 7 after infection. The following samples were then collected for viral load analysis: serum, ovary, testes, brain, kidneys, liver, sciatic nerve and spleen. Following the collection of tissues, the samples were analyzed for viral RNA and infectious ZIKV particles by qRT-PCR and FFA.

Results of qRT-PCR were expressed as genome copies of ZIKV normalized to 18S (ZIKV/18S). The qRT-PCR results for serum were expressed as genomic copies/mL. The FFA, results were reported as log₁₀ FFU/g for most tissues, log₁₀ FFU per tissue for sciatic nerve and ovaries, and log₁₀ FFU/mL for sera.

### Results

Viral Load

Following ZIKV challenge and ZIKV-IG 24 hours post-exposure treatment, the neutralization ability of ZIKV-IG against ZIKV virus replication and dissemination was determined in the serum, brain, sciatic nerve, kidney, liver, spleen, ovary, and testes. Viral load analysis was compared between treatment group and control group for each tissue type using nonparametric exact Wilcoxon Rank-Sum tests at a significance level of α=0.05 (with Bonferroni adjustment for multiplicity).

The results of the study demonstrate that ZIKV-IG administration significantly reduced viral replication and dissemination to target organs in a dose-dependent manner. The highest dose (50 mg/kg) of ZIKV-IG resulted in a significant reduction in viral load in all of the key tissues/organs (brain, kidney, liver, spleen, serum) tested. Although there was a reduction in viral load with mid-dose (10 mg/kg), the extent of reduction was tissue dependent. Significant reduction was observed in organs such as the brain, sciatic nerve, and spleen. There was also a substantial reduction in other organs (kidney, liver) as well. The lower dose ranges tested (2 mg/kg and 0.5 mg/kg) had viral load comparable to the control in most cases.

qRT-PCR

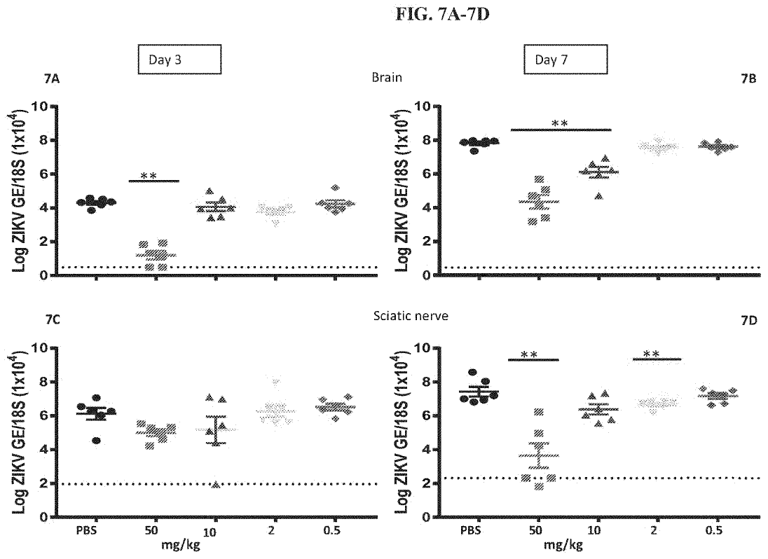

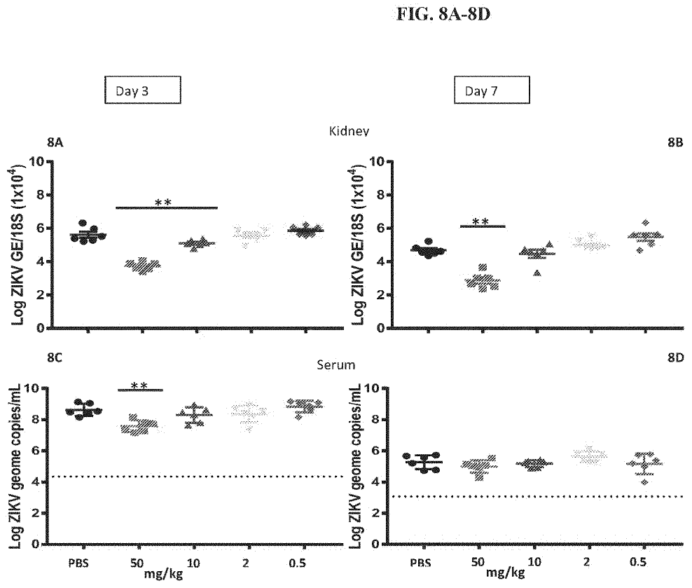

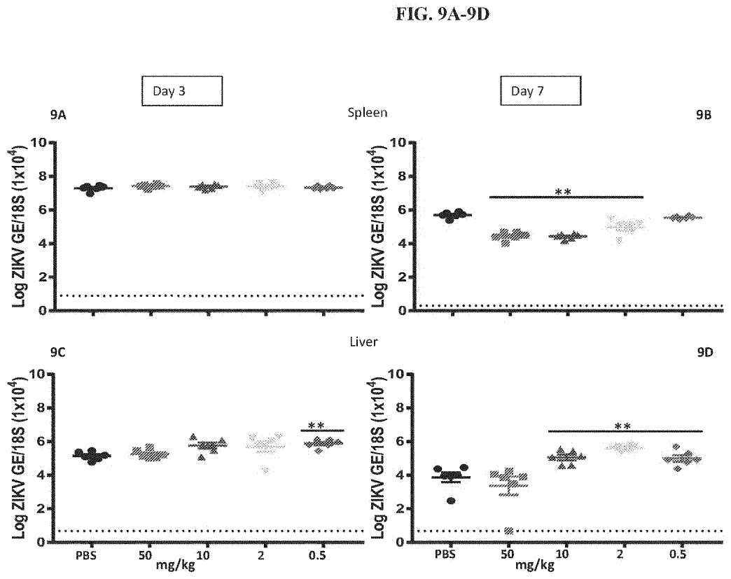

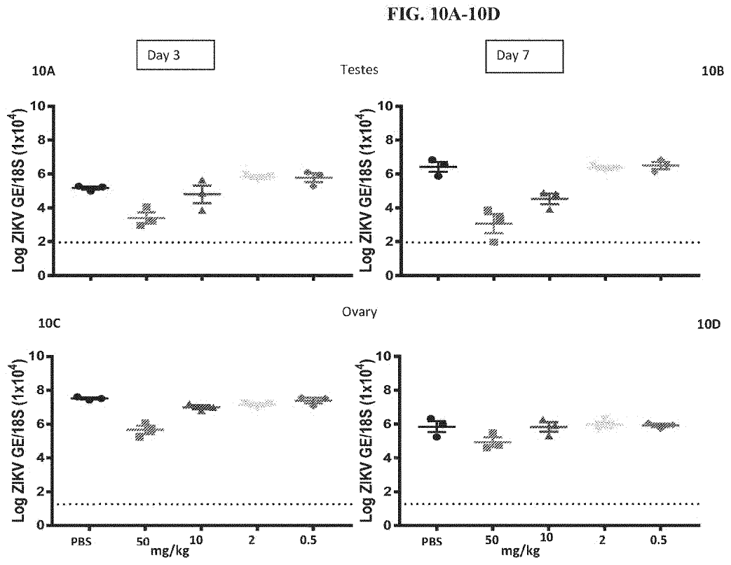

For statistical analysis, genome counts less than the limit of detection were treated as zero values. As shown in FIG. 7A, FIG. 7B, FIG. 7C, FIG. 7D, FIG. 8A, FIG. 8B, FIG. 8C, FIG. 8D, FIG. 9A, FIG. 9B, FIG. 9C, FIG. 9D, FIG. 10A, FIG. 10B, FIG. 10C, and FIG. 10D, the mean viral RNA level was minimal on day 3 and increased by day 7 for the brain, sciatic nerve, and testes, whereas, in tissues such as serum, liver, kidney, and the spleen, the viral RNA levels decreased from day 3 to day 7. Table 6 shows the statistical analysis of these results.

TABLE 6
Statistical Analysis of dose effect of ZIKV-IG versus control
on viral RNA load based on qRT-PCR analysis in tissues.

| | | Day 3 | | Day 7 | |
|---|---|---|---|---|---|
| | | Exact | | Exact | |
| | | Wilcoxon | Bonferroni | Wilcoxon | Bonferroni |
| | | rank-sum p- | adjusted p- | rank-sum p- | adjusted p- |
| | ZIKV-IG | value vs | value vs | value vs | value vs |
| Tissue | Treatment Dose Group | control | control | control | control |
| Brain | ZIKV-IG 50 mg/kg | 0.002* | 0.009* | 0.002* | 0.009* |
| | ZIKV-IG 10 mg/kg | 0.485 | 1.000 | 0.002* | 0.009* |
| | ZIKV-IG 2 mg/kg | 0.015* | 0.061 | 0.093 | 0.372 |
| | ZIKV-IG 0.5 mg/kg | 0.394 | 1.000 | 0.132 | 0.528 |
| Kidney | ZIKV-IG 50 mg/kg | 0.002* | 0.009* | 0.002* | 0.009* |
| | ZIKV-IG 10 mg/kg | 0.009* | 0.035* | 0.937 | 1.000 |
| | ZIKV-IG 2 mg/kg | 0.937 | 1.000 | 0.093 | 0.372 |
| | ZIKV-IG 0.5 mg/kg | 0.240 | 0.961 | 0.015* | 0.061 |
| Liver | ZIKV-IG 50 mg/kg | 0.485 | 1.000 | 0.310 | 1.000 |
| | ZIKV-IG 10 mg/kg | 0.026* | 0.104 | 0.002* | 0.009* |
| | ZIKV-IG 2 mg/kg | 0.065 | 0.260 | 0.002* | 0.009* |
| | ZIKV-IG 0.5 mg/kg | 0.004* | 0.017* | 0.004* | 0.017* |
| Ovaries | ZIKV-IG 50 mg/kg | 0.100 | 0.400 | 0.200 | 0.800 |
| | ZIKV-IG 10 mg/kg | 0.100 | 0.400 | 1.000 | 1.000 |
| | ZIKV-IG 2 mg/kg | 0.100 | 0.400 | 1.000 | 1.000 |
| | ZIKV-IG 0.5 mg/kg | 1.000 | 1.000 | 1.000 | 1.000 |
| Sciatic | ZIKV-IG 50 mg/kg | 0.041* | 0.165 | 0.002* | 0.009* |
| | ZIKV-IG 10 mg/kg | 0.485 | 1.000 | 0.093 | 0.372 |
| | ZIKV-IG 2 mg/kg | 0.937 | 1.000 | 0.009* | 0.035* |
| | ZIKV-IG 0.5 mg/kg | 0.485 | 1.000 | 0.818 | 1.000 |
| Serum | ZIKV-IG 50 mg/kg | 0.004* | 0.017* | 0.310 | 1.000 |
| | ZIKV-IG 10 mg/kg | 0.310 | 1.000 | 0.818 | 1.000 |
| | ZIKV-IG 2 mg/kg | 0.485 | 1.000 | 0.180 | 0.719 |
| | ZIKV-IG 0.5 mg/kg | 0.394 | 1.000 | 1.000 | 1.000 |
| Spleen | ZIKV-IG 50 mg/kg | 0.180 | 0.719 | 0.002* | 0.009* |
| | ZIKV-IG 10 mg/kg | 0.485 | 1.000 | 0.002* | 0.009* |
| | ZIKV-IG 2 mg/kg | 0.310 | 1.000 | 0.004* | 0.017* |
| | ZIKV-IG 0.5 mg/kg | 0.937 | 1.000 | 0.065 | 0.260 |
| Testes | ZIKV-IG 50 mg/kg | 0.100 | 0.400 | 0.100 | 0.400 |
| | ZIKV-IG 10 mg/kg | 0.700 | 1.000 | 0.100 | 0.400 |
| | ZIKV-IG 2 mg/kg | 0.100 | 0.400 | 0.700 | 1.000 |
| | ZIKV-IG 0.5 mg/kg | 0.200 | 0.800 | 1.000 | 1.000 |

*= Significant Wilcoxon rank-sum p-value ≤ 0.05 (Bonferroni adjusted p-value ≤ 0.05)

The viral load analysis for genome copies to 18S was compared between treatment groups and control for each tissue using Bonferroni adjusted nonparametric exact Wilcoxon Rank-Sum tests at a significance level of α=0.05 (Table 6).

The mean viral RNA levels by PCR assay varied with tissue type at the indicated time points in all treatment groups. Serum viremia peaked in all treatment groups on day 3 and declined to low levels by day 7 (FIG. 8). The decline was significant in mice treated with ZIKV-IG 50 mg/kg (group B) compared to the placebo control (Group A) on day 3 (p=0.017; FIG. 8; Table 6). The mean viral RNA load in the control group was low on day 3 but increased by day 7 in the brain, sciatic nerve, and testes (FIG. 7A, FIG. 7B, FIG. 7C, FIG. 7D, FIG. 10A, FIG. 10B, FIG. 10C, and FIG. 10D), while in kidney, spleen and liver, the viral RNA load peaked on day 3 and declined on day 7 (FIGS. 8-9). A significant reduction in viral RNA load was observed in brain from mice treated with 50 mg/kg ZIKV-IG compared to placebo control on days 3 and 7 (p=0.009 and p=0.009, respectively) or 10 mg/kg ZIKV-IG compared to placebo control on day 7 (p=0.009) (FIG. 7, Table 6). Similarly, a reduction in viral RNA load in high dose group (50 mg/kg) in comparison to the placebo was also recorded in other tissues including the sciatic nerve (p=0.009), kidneys (p=0.009 and p=0.009, respectively), and spleen (p=0.009; FIG. 7A, FIG. 7B, FIG. 7C, FIG. 7D, FIG. 8A, FIG. 8B, FIG. 8C, FIG. 8D, FIG. 9A, FIG. 9B, FIG. 9C, and FIG. 9D, Table 6). Mice treated with 50 mg/kg of ZIKV-IG showed a remarkable reduction in the viral RNA load in ovaries and testes on day 3 (FIG. 10).

Mice treated with ZIKV-IG at the 10 mg/kg dose level had a significant reduction in mean viral RNA load when compared to the placebo control in the brain on day 7 (p=0.009), kidneys on day 3 (p=0.035) and spleen on day 7 (p=0.009) (FIG. 7A, FIG. 7B, FIG. 7C, FIG. 7D, FIG. 8A, FIG. 8B, FIG. 8C, FIG. 8D, FIG. 9A, FIG. 9B, FIG. 9C, and FIG. 9D, Table 6).

Mice treated with ZIKV-IG at the 2 mg/kg dose level had a significant reduction in mean viral RNA load in the sciatic nerve (p=0.035), and spleen (p=0.017) on day 7 compared to the placebo control group (FIG. 7A, FIG. 7B, FIG. 7C, FIG. 7D, FIG. 9A, FIG. 9B, FIG. 9C, and FIG. 9D; Table 6). The liver did not show a similar trend of reduction in viral RNA load as observed in other tissue types. Mice treated with ZIKV-IG either at 10 mg/kg (Group C), or 2 mg/kg (Group D) or 0.5 mg/kg (Group E) had significantly increased viral RNA load in liver compared to the placebo control group on day 7 (p=0.009, p=0.009 and p=0.017, respectively (FIG. 9, Table 6).

Focus Forming Assay Analysis

To further characterize viral replication and dissemination into target tissues, focus forming assay (FFA) analysis was used for quantitation of infectious virus particles in target tissues and the data expressed as focus forming units (FFU)/gram of tissue (brain, kidney, spleen, testes and liver), FFU/tissue (ovaries and sciatic nerve) and FFU/mL (serum). Viremia was compared between treatment group and control group for each tissue type using Bonferroni adjusted nonparametric exact Wilcoxon Rank-Sum tests at a significance level of α=0.05.

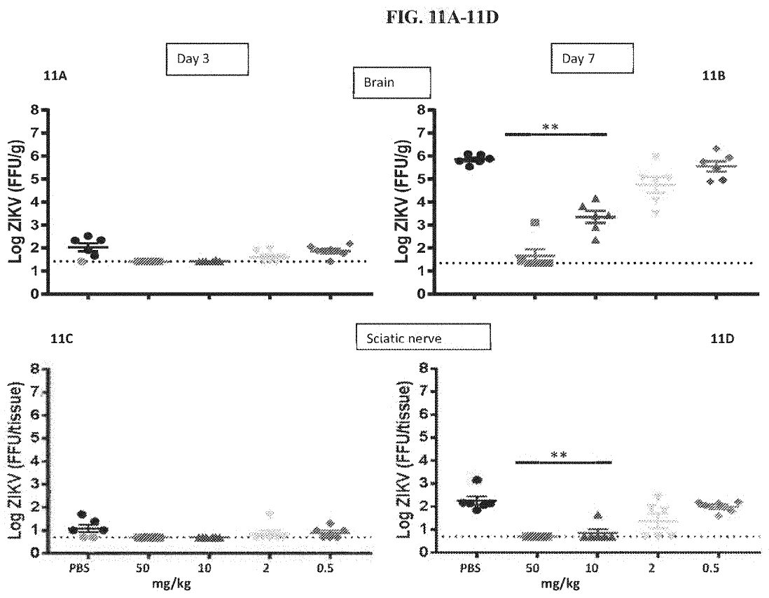

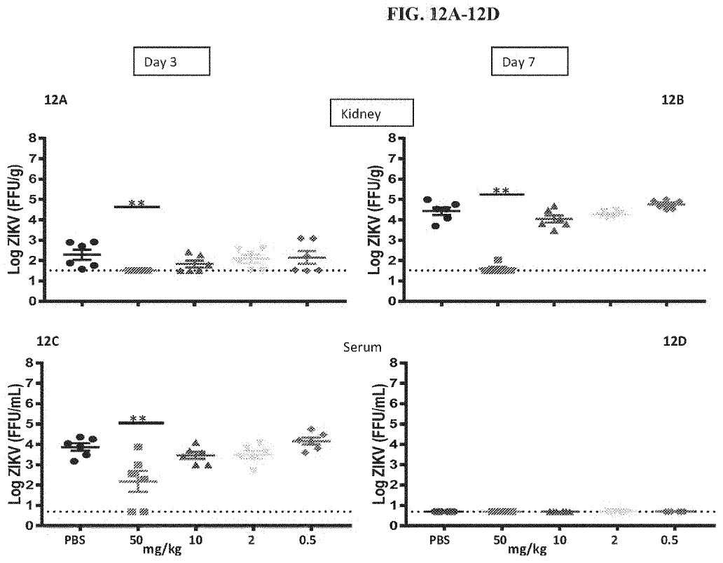

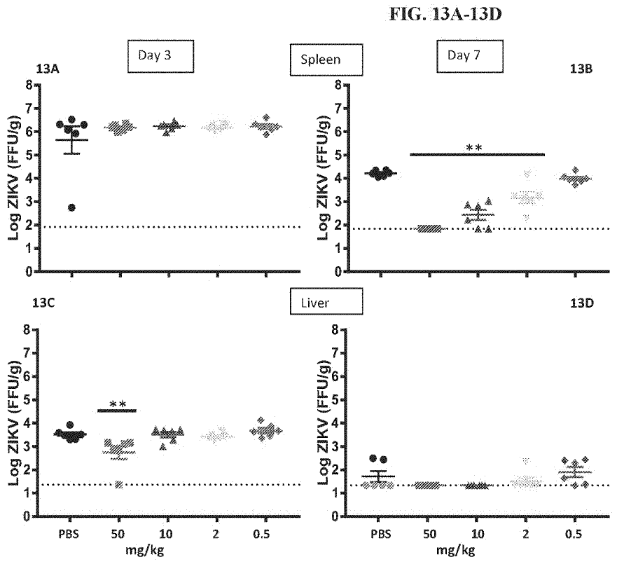

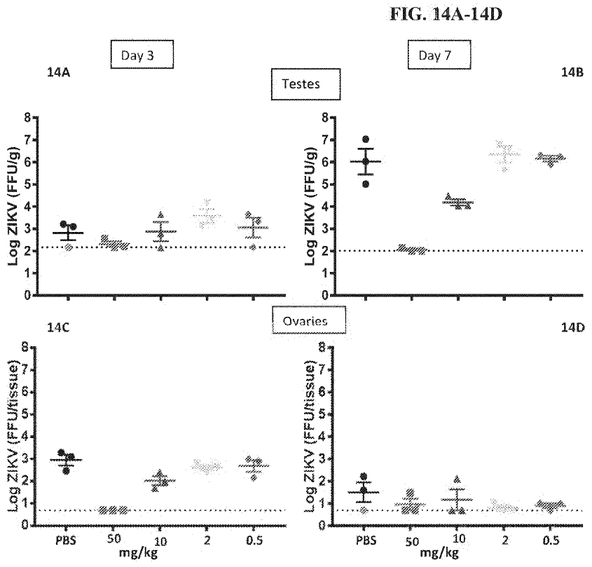

As shown in FIG. 11A, FIG. 11B, FIG. 11C, FIG. 11D, FIG. 12A, FIG. 12B, FIG. 12C, FIG. 12D, FIG. 13A, FIG. 13B, FIG. 13C, FIG. 13D, FIG. 14A, FIG. 14B, FIG. 14C, and FIG. 14D, the viral load in the serum peaked on day 3 in all treatment groups and declined to undetectable levels on day 7. Table 7 shows the statistical analysis of these results. There was a significant reduction in serum viral load (p=0.043) for the group treated with 50 mg/kg of ZIKV-IG on day 3 compared to the placebo group (FIG. 12A, FIG. 12B, FIG. 12C, and FIG. 12D, Table 7). The 50 mg/kg treated mice also had a significant reduction in the brain on day 7 (p=0.009), sciatic on day 7 (p=0.009), kidneys on day 3 (p=0.009) and day 7 (p=0.009) compared to the placebo group (FIG. 11A, FIG. 11B, FIG. 11C, FIG. 11D, FIG. 12A, FIG. 12B, FIG. 12C, and FIG. 12D, Table 7). In addition, there was a significant reduction in the spleen on day 7 (p=0.009) and liver day 3 (p=0.009) compared to the placebo control group (FIG. 13A, FIG. 13B, FIG. 13C, and FIG. 13D, Table 7).

TABLE 7
Statistical analysis of dose effect of ZIKV-IG versus
control based on FFA viral load analysis in tissues.

| | | Day 3 | | Day 7 | |
|---|---|---|---|---|---|
| | | Exact | | Exact | |
| | | Wilcoxon | Bonferroni | Wilcoxon | Bonferroni |
| | | rank-sum p- | adjusted p- | rank-sum p- | adjusted p- |
| | ZIKV-IG | value vs | value vs | value vs | value vs |
| Tissue | Treatment Dose Group | control | control | control | control |
| Brain | ZIKV-IG 50 mg/kg | 0.015* | 0.061 | 0.002* | 0.009* |
| | ZIKV-IG 10 mg/kg | 0.015* | 0.061 | 0.002* | 0.009* |
| | ZIKV-IG 2 mg/kg | 0.102 | 0.407 | 0.026* | 0.104 |
| | ZIKV-IG 0.5 mg/kg | 0.515 | 1.000 | 0.310 | 1.000 |
| Kidney | ZIKV-IG 50 mg/kg | 0.002* | 0.009* | 0.002* | 0.009* |
| | ZIKV-IG 10 mg/kg | 0.180 | 0.719 | 0.128 | 0.511 |
| | ZIKV-IG 2 mg/kg | 0.394 | 1.000 | 0.370 | 1.000 |
| | ZIKV-IG 0.5 mg/kg | 0.240 | 0.961 | 0.660 | 1.000 |
| Liver | ZIKV-IG 50 mg/kg | 0.002* | 0.009* | 0.455 | 1.000 |
| | ZIKV-IG 10 mg/kg | 0.818 | 1.000 | 0.455 | 1.000 |
| | ZIKV-IG 2 mg/kg | 0.589 | 1.000 | 0.455 | 1.000 |
| | ZIKV-IG 0.5 mg/kg | 0.310 | 1.000 | 0.275 | 1.000 |
| Ovaries | ZIKV-IG 50 mg/kg | 0.100 | 0.400 | 0.400 | 1.000 |
| | ZIKV-IG 10 mg/kg | 0.100 | 0.400 | 0.700 | 1.000 |
| | ZIKV-IG 2 mg/kg | 0.400 | 1.000 | 0.400 | 1.000 |
| | ZIKV-IG 0.5 mg/kg | 0.400 | 1.000 | 0.500 | 1.000 |
| Sciatic | ZIKV-IG 50 mg/kg | 0.061 | 0.242 | 0.002* | 0.009* |
| | ZIKV-IG 10 mg/kg | 0.061 | 0.242 | 0.002* | 0.009* |
| | ZIKV-IG 2 mg/kg | 0.197 | 0.788 | 0.054 | 0.216 |
| | ZIKV-IG 0.5 mg/kg | 0.448 | 1.000 | 0.584 | 1.000 |
| Serum | ZIKV-IG 50 mg/kg | 0.011* | 0.043* | 1.000 | 1.000 |
| | ZIKV-IG 10 mg/kg | 0.169 | 0.675 | 1.000 | 1.000 |
| | ZIKV-IG 2 mg/kg | 0.240 | 0.961 | 1.000 | 1.000 |
| | ZIKV-IG 0.5 mg/kg | 0.394 | 1.000 | 1.000 | 1.000 |
| Spleen | ZIKV-IG 50 mg/kg | 1.000 | 1.000 | 0.002* | 0.009* |
| | ZIKV-IG 10 mg/kg | 0.937 | 1.000 | 0.002* | 0.009* |
| | ZIKV-IG 2 mg/kg | 0.937 | 1.000 | 0.009* | 0.035* |
| | ZIKV-IG 0.5 mg/kg | 0.699 | 1.000 | 0.065 | 0.260 |
| Testes | ZIKV-IG 50 mg/kg | 0.500 | 1.000 | 0.100 | 0.400 |
| | ZIKV-IG 10 mg/kg | 1.000 | 1.000 | 0.100 | 0.400 |
| | ZIKV-IG 2 mg/kg | 0.200 | 0.800 | 1.000 | 1.000 |
| | ZIKV-IG 0.5 mg/kg | 0.500 | 1.000 | 1.000 | 1.000 |

*= Significant Wilcoxon rank-sum p-value ≤ 0.05 (Bonferroni adjusted p-value ≤ 0.05)

The reduction in tissue viral load was also observed with the ZIKV-IG at 10 mg/kg dose level. A significant reduction was observed compared to the placebo control on day 7 in the brain (p=0.009), sciatic nerve (p=0.009) and spleen (p=0.009; FIG. 11A, FIG. 1B, FIG. 1C, FIG. 111, FIG. 13A, FIG. 13B, FIG. 13C, and FIG. 131D; Table 7). In addition, the ZKV-IG at 2 mg/kg dose level showed significant reduction in viral load in the spleen on day 7(p=0.035; FIG. 13A, FIG. 13B, FIG. 13C, and FIG. 131D; Table 7).

Finally, viral load in testes at day 7 was reduced in both ZIKV-IG 50 mg/kg and 10 mg/kg treated mice groups compared to control. Similarly, viral load in ovaries at day 3 was reduced in the ZIKV-IG 50 mg/kg group in the viral load in the ovaries on day 3 compared to control (FIG. 14A, FIG. 14B, FIG. 14C, and FIG. 14D; Table 7).

### Conclusion

These results indicate a significant treatment benefit with ZIKV-IG in terms of controlling viral replication in target tissues. At 50 mg/kg dose levels, ZIKV-IG was able to reduce viral RNA levels significantly in most tissues including the brain, kidney, serum, sciatic nerve and spleen. At 10 mg/kg dose level, significant reduction in comparison to control was noted for the following tissues: the brain, kidney, and spleen.

Similar to qRT-PCR analysis, the reduction in infectious virus levels as measured by FFA was dose-dependent. In most tissues tested (brain, kidney, liver, sciatic nerve, serum, and spleen), the viral load reduction was statistically significant with 50 mg/kg dose in comparison to control. The extent of reduction varied with the tissues (0.77 log to 4.2 log reduction), and there was a remarkable reduction in both ovaries and testes. Mice treated with the 10 mg/kg dose also exhibited a significant reduction in viral load in comparison to control for the following tissues: the brain, sciatic nerve, and spleen. Furthermore, qRT-PCR analysis demonstrated a higher viral RNA load in the liver at 10, 2, and 0.5 mg/kg when compared to the control group. However, the infectious viral load analysis of the liver showed no significant difference in the viral load between lower doses (2 to 0.5 mg/kg) and the control group by FFA.

By both analyses, there was a noticeable decrease in viral load in the immune privilege tissues such as the brain, and testes, especially with the 50 mg/kg dose. These results demonstrate that the ZIKV-IG treatment prevents virus dissemination and persistence.

Collectively, these results demonstrated virus neutralization by ZIKV-IG in a dose-related fashion consistent with its mechanism of action. The 50 mg/kg dose was highly effective in improving survival, controlling viral replication and dissemination in target tissues. These results support the use of ZIKV-IG as a therapeutic candidate for Zika infection in humans.

### Example 4: Dose-Ranging Study of ZIKV-IG Polyclonal Antibodies in a Lethal Model of Zika Virus Infection in Ifnar1−/− Mice—Pathological Analysis

A study was designed to analyze the effect of ZIKV-IG on virus-induced tissue pathology in target tissues.

### Study

This study was designed to evaluate the efficacy of ZIKV-IG in controlling virus-induced tissue damage and persistence in target tissues from Ifnar1−/− mice infected with a lethal amount of ZIKV. ZIKV-IG is an immunoglobulin G (IgG) product containing neutralizing antibody to ZIKV which was formulated as a single-use vial in sterile liquid stabilized with 10% Maltose and 0.03% polysorbate 80 (pH between 5.0 and 6.5) and free of preservatives.

Mice were bred and maintained in an approved facility following protocols approved by an Animal Care and Use Committee. Mice were separated based on sex and randomized by weights, and blinded into 4 dose/treatment study groups (A, B1, B2 and C) including the control (Table 8).

Mice were challenged via IV route (retro-orbital injection) inoculation with a lethal dose (1.0×10³ FFU/mouse) of Asian ZIKV lineage FSS13025 within +/−2 hours after preparation of the challenge virus. Each group was treated 24+/−2 hours after ZIKV infection with one of 2 different doses of ZIKV-IG (50 mg/kg and 0.5 mg/kg). The control group was treated with PBS. The 50 mg/kg treatment group was tested at 7 and 21 days post-infection and the 0.5 mg/kg treatment group was tested at 7 days post-infection. Necropsy was performed and the brain, kidney, liver, spleen, spinal cord, sciatic nerve, ovary and testes were harvested for histopathology at 7 and 21 days post-infection. In addition, immunohistochemistry was conducted on selected tissues that included the brain, liver, ovaries and testes on days 7 and 21. Immunohistochemistry data for ZIKV antigen detection for each tissue type were summarized using descriptive statistics for each animal and each tissue assessed.

TABLE 8
Treatment Groups and Clinical Parameter to Monitor ZIKV Challenge in Ifnar1^{-/-} Mice Model

| | | POST-INFECTION TREATMENT GROUP | | | | | |
|---|---|---|---|---|---|---|---|
| | | | ZIKV-IG | | | | |
| | | | (54 mg/niL) | Volume | Final | | |
| | | ZIKV | Treatment Doses | ZIKV-IG/ | Dilution | Harvest | Clinical |
| | | Infection | (IV, 24 hrs | Mouse at | Scheme for | Date | Parameters |
| Group | Gender | (IV Route) | Post-Infection) | 54 mg/mL | Product | (Post-Infection) | Assessed |
| A | 4 Female | ZIKV | PBS | — | — | 7 | The following |
| | 4 Male | infection with | | | | | tissues will be |
| B1 | 4 Female | strain | 50 mg/kg | 18.5 gL | 1:5 | 7 | harvested - |
| | 4 Male | FSS13025, at | | | (Solution 1) | | Histopathology: |
| B2 | 4 Female | dose of 1.0 × | 50 mg/kg | 18.5 gL | 1:5 | 21 | Brain, Kidney, |
| | 4 Male | 10³ | | | (Solution 1) | | Liver, Spleen, |
| C | 4 Female | FFU/animal. | 0.5 mg/kg | 0.19 gL | 1:500 | 7 | Spinal cord, |
| | 4 Male | Administered | | | (Solution 2) | | Sciatic nerve, |
| | | intravenously | | | | | Ovaries and |
| | | (Retro-orbital | | | | | Testis. |
| | | injection) | | | | | Immunohisto- |
| | | | | | | | chemistry: |
| | | | | | | | Brain, Liver, |
| | | | | | | | Ovaries and |
| | | | | | | | Testis. |

### Results

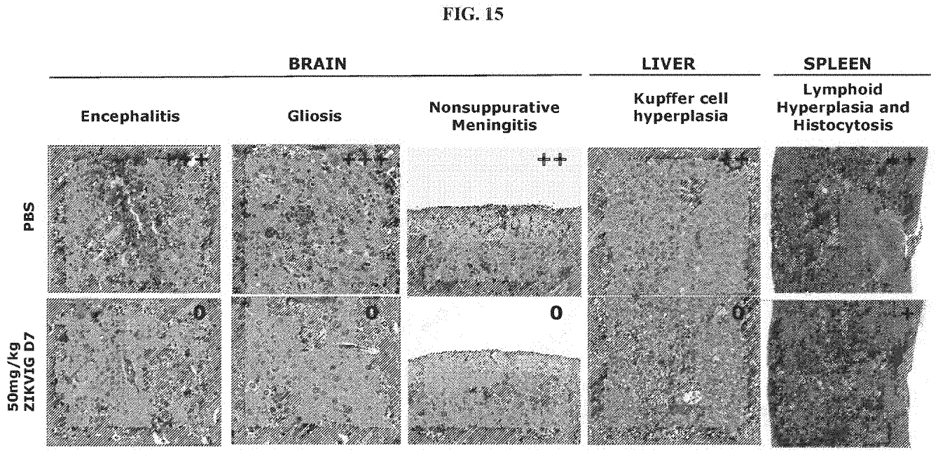

Tissue harvest and histopathology was performed on a total of 31 (4 females+4 males/group) surviving animals euthanized on days 7 or 21. Harvested tissues were examined by board certified pathologist and the presence of microscopic findings were graded semi-quantitatively for each lesion. The grading scheme used was: Grade 0: No lesion; Grade+: Low or light lesion; Grade++: Moderate or slightly discernible lesion; and Grade+++: Severe or discernible lesion affecting a large area of the tissue. Semi-quantitative histopathology scores (0, +, ++ and +++) were converted to numeric scores (0, 1, 2 and 3, respectively) and averaged to present a mean score for each tissue type, treatment group and tissue harvest date post-virus challenge (Table 9). For tissues where microscopic lesions were observed, lesion severity tended to be markedly lower in the high dose (50 mg/kg) ZIKV-IG treatment groups (B1 and B2) compared to control animals (group A). Slight microscopic lesion severity was observed in the brain and liver of high dose animals (group B2) sacrificed on 21 days compared to high dose animals (group B1) sacrificed 7 days after virus challenge. This could be due to the impaired ability of the Ifnar1^{-/-} mouse strain to mount a proper immune response. Microscopic lesion severity was similar in the tissues analyzed in low dose (group C) ZIKV-IG treated mice compared to controls (FIG. 15).

TABLE 9
Summary of Day 7 and Day 21 histopathology findings for tissues of Treatment Groups

| | | Most Frequently Observed Severity (Average Severity) | | | | | | | |
|---|---|---|---|---|---|---|---|---|---|
| | | | | | | Group B1 | | Group B2 | |
| | | Group A | | Group C | | ZIKVIG | | ZIKVIG | |
| | | Control | | ZIKVIG | | (50 mg/kg) | | (50 mg/kg) | |
| Tissue | Lesion Type | (PBS) | | (0.5 mg/kg) | | Day 7 | | Day 21 | |
| Brain | Nonsuppurative | ++ | (2.43) | ++ | (2.13) | 0 | (0.38) | ++ | (1.75) |
| | encephalitis | | | | | | | | |
| | Gliosis | ++ | (2.00) | +/++ | (1.50) | 0/+ | (0.50) | + | (0.75) |
| | Nonsuppurative meningitis | ++ | (1.86) | + | (1.13) | 0 | (0.13) | + | (1.25) |
| | Neuronal Necrosis | + | (1.43) | ++ | (1.25) | 0 | (0.38) | 0 | (0.38) |
| | Malacia | + | (0.71) | + | (1.25) | 0 | (0.13) | 0 | (0.13) |
| | Hemorrhage | 0 | (0.29) | 0 | (0.00) | 0 | (0.00) | 0 | (0.00) |
| Liver | Kupffer Cell Hyperplasia | + | (1.29) | +++ | (2.63) | 0 | (0.25) | + | (0.75) |
| | Microvesicular | 0 | (0.43) | 0 | (0.13) | + | (1.00) | + | (1.38) |
| | Vacuolization | | | | | | | | |
| | Minimal multifocal | 0 | (0.00) | 0 | (0.00) | 0 | (0.00) | 0 | (0.38) |
| | necrotizing hepatitis | | | | | | | | |
| Spleen | Lymphoid Hyperplasia | + | (1.29) | + | (1.13) | + | (1.00) | 0 | (0.63) |
| | Red/white histiocytosis | + | (1.29) | + | (1.13) | + | (1.00) | 0 | (0.63) |
| Spinal | Nonsuppurative cervical | 0 | (0.29) | 0/+ | (0.50) | 0 | (0.13) | + | (0.63) |
| Cord | myelitis | | | | | | | | |
| | Nonsuppurative thoracic | 0 | (0.29) | 0 | (0.13) | 0 | (0.13) | 0 | (0.25) |
| | myelitis | | | | | | | | |
| | Nonsuppurative lumbar | 0 | (0.29) | 0 | (0.38) | 0 | (0.00) | 0 | (0.00) |
| | myelitis | | | | | | | | |
| Testes | Neutrophilic Orchitis | 0 | (0.00) | 0 | (0.00) | 0 | (0.00) | 0 | (0.50)* |

*Single animal in B2 group scored ‘+’

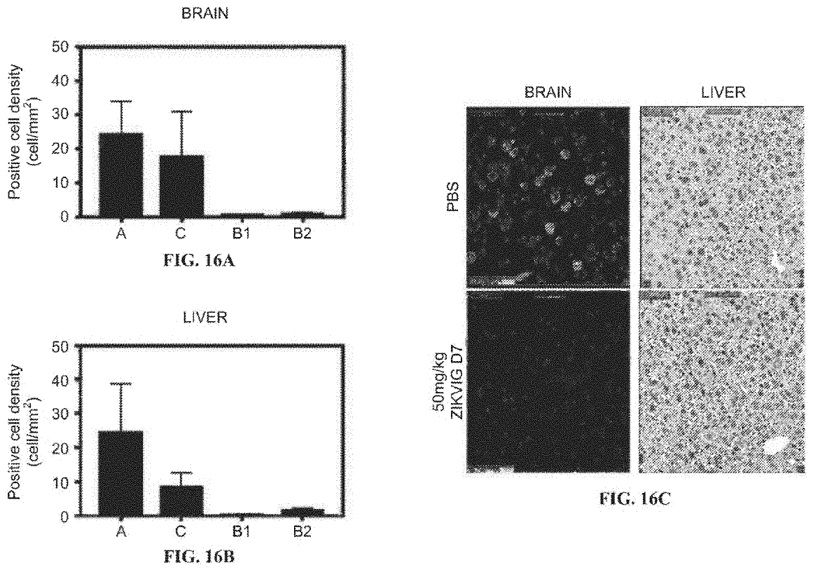

The brain, liver, ovaries and testes of treatment groups were harvested at indicated time points for fluorescence immunohistochemistry. However, the data from ovaries and testes was uninterpretable due to auto-fluorescence attributed to ovarian and testicular proteins and dense secretions. As such, no true positive immunoreactivity for ZIKV antigens was detected in these two tissue sections. Positive immunoreactivity was detected in the anterior forceps region of the brain and in the liver in sinusoidal endothelial cells. Quantitative analysis indicates a marked reduction in immunoreactivity in both tissues at the higher dose level (group B1 and B2) in terms of mean positive cell density compared to control (group A). The lower dose level (group C) showed a more moderate reduction in mean positive cell density compared to control (group A). See FIG. 16A and FIG. 16B.

This study demonstrates the benefit of ZIKV-IG post-exposure prophylactic treatment in terms of controlling virus-induced tissue damage and persistence in target tissues. A 50 mg/kg dose conferred the greatest benefit while the low dose (0.5 mg/kg) showed moderate reduced virus-induced tissue damage and ZIKV persistence in target tissues in a post-exposure prophylaxis scenario compared to controls.

### Example 5: ZIKV-IG Antibody-Dependent Enhancement of ZIKV in Human Primary Macrophages

To determine whether or not ZIKV-IG can induce ADE in ZIKV infected monocyte-derived macrophages (MDMs), the following study was performed.

### Study

ZIKV (strain FSS13025) was pre-incubated at a multiplicity of infection (MOI) of 1 with either PBS (placebo control), human dengue immune serum (ARAVINDA serum, positive control), or with serial dilutions of either Gamunex (negative control) or ZIKV-IG product (Table 10).

TABLE 10
Summary of test and control article concentrations

| Treatments | Final Antibody concentrations | | | | | |
|---|---|---|---|---|---|---|
| ZIKV-IG | 0.00032 | 0.0016 | 0.008 | 0.04 | 0.2 | 1 |
| | mg/mL | mg/mL | mg/mL | mg/mL | mg/mL | mg/mL |
| Gamunex | 0.0016 | 0.008 | 0.04 | 0.2 | 1 | 5 |
| | mg/mL | mg/mL | mg/mL | mg/mL | mg/mL | mg/mL |

| | |
|---|---|
| ARAVINDA | 0.6% human dengue serum |
| serum | |
| PBS | Not applicable |

Test Material for all treatments was ZIKV (strain (FSS13025) at a MOI1.
Test System for all treatments were monocyte derived macrophages (4 × 10⁵ cells/well)

To determine if ZIKV-IG can induce ADE of ZIKV infection in MDMs, blood was collected from three healthy human donors and peripheral blood mononuclear cells (PBMCs) were isolated. Monocytes were subsequently isolated from the PBMCs using MACS magnetic beads, cultured and differentiated into macrophages.

ZIKV strain FSS13025 (MOI=1) was incubated at 37° C. for 30 minutes with either PBS (placebo control), 0.6% human dengue immune serum at single dilution (ARAVINDA serum, positive control), one of five-fold serial dilution concentrations of Gamunex (negative control, Lot #26NC421) or one of five-fold serial dilution concentrations of ZIKV-IG (Table 10). Prior to addition to MDMs (4×10⁵ cells/well) at a MOI=1.

After incubation with virus, either virus/PBS, virus/dengue immune serum, virus/Gamunex or virus/ZIKV-IG preparations were added on MDMs and incubated 2 hours at 37° C. with rocking. After intensive washes with 1×PBS, and incubation for an additional 22 hours, cells were washed with FACS buffer (1×PBS w/3% FBS (GEMINI BIO-PRODUCT, Cat #100-0106)+1 mM EDTA (Invitrogen, Cat #15575-038)) and fixed/permeabilized using cytofix/cytoperm solution (BD Bioscience, Cat #51-2090ZK) at 4° C. for 30 minutess. Cells were then washed three-times in 1× Perm/Wash buffer solution (10×Perm/Wash Buffer (BD Bioscience, Cat #51-2091KZ) diluted in Molecular grade water) and stained with a fluorescein isothiocyanate (FITC) labeled pan-flavivirus specific 4G2 mAb (Clone D1-4G2-4-14, hybridoma from ATCC, antibody made by BIOXCELL, Lot #630816D1) at 4° C. for 20 minutes. After staining, cells were washed two times with 1×Perm/Wash buffer solution and another one-time wash with FACS buffer. Thereafter, cells were resuspended in 200 μL FACS buffer and analyzed on a BD LSR II Flow Cytometer (BD Bioscience Clontech, USA). The percentage of positively stained cells with 4G2 was determined using FlowJo FACS analysis software (FlowJo LLC version 10.02, USA).

### Results

Cells were collected and assayed in two groups of three patients each. For both groups of patients, exposure of cells to ZKV-G enhanced ZKV infection compared to those cells exposed to Gamunex. Response peaked in five of six samples at the 0.008 mg/mL ZIKV-IG and at 0.00016 mg/mL ZIKV-G in the sixth sample (Tables 1 and 12). Peak enhancement compared to the PBS controls was substantially higher in the first group of three patients evaluated (Table 11) compared to the second group (Table 12). The Gamunex isotype control antibody did not enhance ZIKV infection compared to PBS control at the concentrations tested (0.00032 mg/mL to mg/mL). ARAVINDA serum (positive control) demonstrated enhancement of ZIKV infection in all donors for which it was tested.

This study demonstrates that pre-treatment of MDMs with ZIKV-IG in a defined dose range can enhance subsequent ZIKV infection.

TABLE 11
ZIKV Uptake Measured by FACS and Expressed as Fold Increase Over PBS Control
in Monocyte Derived Macrophages Obtained from Peripheral Blood Mononuclear
Cell Samples of Three Donors (EB54-138, EB55-198 and EB56-159)

| | | Gamunex | | | | | | | | |
|---|---|---|---|---|---|---|---|---|---|---|
| Antibody/Human | | (Isotype Control) | | | ARAVINDA Serum | | | ZIKV-IG | | |
| Dengue Serum | | EB54- | EB55- | EB56- | EB54- | EB55- | EB56- | EB54- | EB55- | EB56- |
| Concentration | | 138 | 198 | 159 | 138 | 198 | 159 | 138 | 198 | 159 |
| 0.6% | | — | — | — | — | 4.54 | 19.37 | — | — | — |
| 1 | mg/mL | 0.63 | 0.41 | 0.62 | — | — | — | 0.74 | 0.51 | 0.83 |
| 0.2 | mg/mL | 0.72 | 0.59 | 0.90 | — | — | — | 0.48 | 0.71 | 00.77 |
| 0.04 | mg/mL | 0.62 | 0.52 | 1.07 | — | — | — | 2.03 | 1.22 | 3.12 |
| 0.008 | mg/mL | 0.79 | 0.39 | 1.48 | — | — | — | 13.11 | 11.75 | 27.24 |
| 0.0016 | mg/mL | 1.05 | 0.36 | 0.96 | — | — | — | 10.20 | 13.11 | 24.76 |
| 0.00032 | mg/mL | 0.84 | 0.37 | 0.96 | — | — | — | 6.02 | 3.41 | 11.49 |

TABLE 12
ZIKV Uptake Measured by FACS and Expressed as Fold Increase Over PBS Control
in Monocyte Derived Macrophages Obtained from Peripheral Blood Mononuclear
Cell Samples of Three Donors (EB66-227, EB67-216 and EB68-176)

| | | Gamunex | | | | | | | | |
|---|---|---|---|---|---|---|---|---|---|---|
| Antibody/Human | | (Isotype Control) | | | ARAVINDA Serum | | | ZIKV-IG | | |
| Dengue Serum | | EB66- | EB67- | EB68- | EB66- | EB67- | EB68- | EB66- | EB67- | EB68- |
| Concentration | | 227 | 216 | 176 | 227 | 216 | 176 | 227 | 216 | 176 |
| 0.6% | | — | — | — | 2.99 | 3.83 | 5.35 | — | — | — |
| 5 | mg/mL | 0.82 | 0.90 | 2.76 | — | — | — | — | — | — |
| 1 | mg/mL | 0.61 | 0.42 | 1.60 | — | — | — | 0.82 | 2.13 | 0.65 |
| 0.2 | mg/mL | 0.45 | 0.52 | 1.19 | — | — | — | 0.58 | 0.91 | 0.87 |
| 0.04 | mg/mL | 0.64 | 0.52 | 1.46 | — | — | — | 1.53 | 2.28 | 5.27 |
| 0.008 | mg/mL | 0.49 | 0.49 | 1.03 | — | — | — | 3.63 | 6.37 | 5.60 |
| 0.0016 | mg/mL | 0.38 | 0.28 | 0.63 | — | — | — | 0.81 | 2.05 | 3.41 |
| 0.00032 | mg/mL | — | — | — | — | — | — | 0.53 | 0.93 | 1.44 |

### Example 6: Efficacy of Zika Polyclonal Antibodies in the ZIKV Pregnancy Infection Model in Ifnar1−/− Mice

To evaluate the efficacy of ZIKV-IG in ZIKV infected pregnant Ifnar1−/− mice in a pre-exposure prophylaxis setting, the following study is performed.

### Study

This study was designed as a randomized study to evaluate the prophylactic efficacy of ZIKV-IG at preventing vertical transmission of ZIKV in pregnant Ifnar1^{-/-} female mice mated to wild type males. Preliminary determination of pregnancy was done by identification of a vaginal plug. For purposes of treatment, challenge and sacrifice the date of identification of a vaginal plug was defined as being embryonic day 0.5 (E0.5) for that animal. Treatment was at E6.5 and challenge with ZIKV strain PRVABC59 was at E7.5. Outcome measures included fetal size and weight, fetal viral RNA load (fetal brain or head), and maternal viral RNA load (serum and select tissues). Technicians assessing fetal size and weight were blinded.

Fifty-five Ifnar1−/− female mice were mated with 20 C57BL/6J WT male mice. The first 36 females with vaginal plug detection were separated from male mice and randomized into treatment groups based on the order of observation of the vaginal plug. At E6.5 pregnant mice were administered ZIKV-IG at either 25 mg/kg (n=11) or 100 mg/kg, (n=7) or with PBS for control animals (n=11) in order to ensure a total of six confirmed pregnant animals were in each study group (Table 13). Test and Control Article administration was by the i.v. (retro-orbital route). At E7.5 pregnant mice were challenged with 1.0×10⁴ FFU of ZIKV strain PRVABC59 in 200 μL of PBS with 10% FBS i.v. (retro-orbital) within 2 hrs of preparation of the challenge virus. Pregnant mice were sacrificed at E14.5 and pregnancy confirmed by observation of embryonic development or residual placenta if fetal reabsorption had occurred. Only the first six confirmed pregnant mice per group were used for the study. Upon confirmation of pregnancy fetuses were harvested for fetal outcome measurements including fetal size, fetal weight, and viral RNA load in fetal head and fetal body.

TABLE 13
Treatment Groups and Clinical Parameters Assessed at Time of Fetal Harvest

| | Animal Number | Treatment | Virus challenge | Clinical parameter |
|---|---|---|---|---|
| Group | and Gender | at E6.5^{a} | at E7.5^{a} | assessments at E14.5 |
| A | 6 (all | PBS (200 μL) | ZIKV strain | Fetal size and weight |
| | females) | | PRVABC59 | Fetal head and body |
| B1 | 6 (all | ZIKV-IG (25 | (1.0 × 10⁴ FFU) | viral load (by qRT-PCR) |
| | females) | mg/kg) | | Maternal serum, brain, |
| B2 | 6 (all | ZIKV-IG (100 | | and spleen viral load |
| | females) | mg/kg) | | (by qRT-PCR) |
| | | | | Placenta with decidua |
| | | | | viral load (by qRT- |
| | | | | PCR) |

^{a}Test Article, Control Article and Virus Challenge were all administered i.v., (retro-orbital route) in a final volume of 200 μL.

### Results

Fetal size in the 100 mg/kg ZIKV-G treatment group compared to PBS controls was the primary endpoint for this study and was significantly greater in the 100 mg/kg ZIKV-IG treated group compared to PBS controls (Table 14, Table 15, p 0.001) indicating that this dose of ZIKV-IG administered as a pre-exposure prophylactic is effective at improving fetal outcomes in this model.

TABLE 14
Descriptive statistics of fetal size (mm)
at E14.5 by pre-exposure treatment group

| | | | Median (mm) | |
|---|---|---|---|---|
| Treatment | Number of | | (25th-75th | Min, |
| Group | Fetuses | Mean (SD) (mm) | percentile) | Max (mm) |
| Group A | 42 | 5.28 (0.80) | 5.3 (4.7, 5.8) | 3.2, 7.1 |
| Group B1 | 39 | 5.58 (1.07) | 5.5 (5.0, 6.0) | 3.3, 9.5 |
| Group B2 | 45 | 7.24 (2.66) | 6.3 (5.6, 7.3) | 4.2, 13.3 |

TABLE 15
Two-sample Wilcoxon Rank-Sum Test
to compare fetal size at E14.5

| Treatment Group | p-value compared to Group A^{a} |
|---|---|
| Group B1 | 0.220 |
| Group B2 | <0.001* |

^{a}Wilcoxon Rank-Sum Test with normal approximation.
*= Statistically significant result at α = 0.05

Exploratory analysis to assess fetal weight as another metric of fetal outcomes showed that ZIKV-IG improved fetal outcomes compared to PBS controls at both the 100 mg/kg and 25 mg/kg ZIKV-IG doses tested (p<0.001 and p=0.007, respectively Table 16 and Table 17). In contrast, fetal size was not significantly different in the 25 mg/kg ZIKV-IG treatment group compared to controls. Despite this, overall fetal size and weight data suggest that ZIKV-IG improves fetal outcomes when administered at a sufficiently high dose.

TABLE 16
Descriptive statistics of fetal weight
(g) by pre-exposure treatment group

| | | | Median (g) | |
|---|---|---|---|---|
| Treatment | Number of | | (25th-75th | Min, |
| Group | Fetuses | Mean (SD) (g) | percentile) | Max (g) |
| Group A | 42 | 0.082 (0.025) | 0.08 (0.07, 0.10) | 0.02, 0.18 |
| Group B1 | 39 | 0.102 (0.035) | 0.09 (0.08, 0.12) | 0.05, 0.22 |
| Group B2 | 45 | 0.119 (0.069) | 0.11 (0.09, 0.13) | 0.01, 0.30 |

TABLE 17
Two-sample Wilcoxon Rank-Sum Test
to compare fetal weight at E14.5

| Treatment Group | p-value compared to Group A^{a} |
|---|---|
| Group B1 | 0.007* |
| Group B2 | <0.001* |

^{a}Wilcoxon Rank-Sum Test with normal approximation.
*= Statistically significant result at α = 0.05

Additional analysis of fetal phenotypes was conducted to provide further support for the efficacy of ZIKV-IG at improving fetal outcomes and to demonstrate that these correspond to the improvements in fetal size and weight seen with ZIKV-IG treatment, which in the case of reabsorbed fetuses were actually the size and weight of the placental and any residual fetus. This fetal phenotype analysis was consistent with fetal outcomes as assessed by fetal size and weight measurements, demonstrating significant improvement (p≤0.012, Tables 18, 19, 20 and 21) in fetal outcomes associated with the 100 mg/kg ZIKV-IG treatment group. Significant differences in fetal phenotypes were not observed between the 25 mg/kg ZIKV-IG treatment group and controls.

TABLE 18
Analysis of fetal phenotype by treatment group - all 4 phenotypes

| | Number (%) of Fetuses | | | | | p-value |
|---|---|---|---|---|---|---|
| | | Total | Intermediate | | | Compared |
| | | Growth | Growth | | | to Group |
| Group | Reabsorbed | Restricted | Restricted | Normal | Total | A^{a} |
| Group A | 42 (100) | 0 (0) | 0 (0) | 0 (0) | 42 | — |
| Group B1 | 38 (97) | 0 (0) | 1 (3) | 0 (0) | 39 | 0.482 |
| Group B2 | 33 (73) | 3 (7) | 2 (4) | 7 (16) | 45 | <0.001 |

^{a}Two-sided Fisher's exact test, unadjusted for multiplicity

TABLE 19
Analysis of fetal phenotype by treatment group -
combining 2 growth restricted phenotypes

| | Number (%) of Fetuses | | | | p-value |
|---|---|---|---|---|---|
| | | “Growth | | | Compared to |
| Group | Reabsorbed | Restricted” | Normal | Total | Group A^{a} |
| Group A | 42 (100) | 0 (0) | 0 (0) | 42 | — |
| Group B1 | 38 (97) | 1 (3) | 0 (0) | 39 | 0.482 |
| Group B2 | 33 (73) | 5 (11) | 7 (16) | 45 | <0.001 |

^{a}Two-sided Fisher's exact test, unadjusted for multiplicity

TABLE 20
Analysis of fetal phenotype by treatment group -
reabsorbed versus 3 other phenotypes

| | Number (%) of Fetuses | | | p-value |
|---|---|---|---|---|
| | | “Non- | | Compared to |
| Group | Reabsorbed | reabsorbed” | Total | Group A^{a} |
| Group A | 42 (100) | 0 (0) | 42 | — |
| Group B1 | 38 (97) | 1 (3) | 39 | 0.482 |
| Group B2 | 33 (73) | 12 (27) | 45 | <0.001 |

^{a}Two-sided Fisher's exact test, unadjusted for multiplicity.

TABLE 21
Analysis of fetal phenotype by treatment
group - normal versus 3 other phenotypes

| | | | | p-value |
|---|---|---|---|---|
| | Number (%) of Fetuses | | | Compared to |
| Group | Normal | “Non-normal” | Total | Group A^{a} |
| Group A | 0 (0) | 42 (100) | 42 | — |
| Group B1 | 0 (0) | 39 (100) | 39 | N/A |
| Group B2 | 7 (16) | 38 (84) | 45 | 0.012 |

^{a}Two-sided Fisher's exact test, unadjusted for multiplicity.

Viral RNA load in placenta/decidua was significantly reduced by both the 100 mg/kg and 25 mg/kg ZIKV-IG doses tested compared to controls (p<0.001 Table 22 and Table 23), as was maternal viral RNA load in spleen and brain (p=0.005, Tables 24, 25, and 27). The significant reduction of placenta/decidua and maternal tissue viral RNA load in the 25 mg/kg ZIKV-IG group was not reflected in improvements in fetal outcomes. Maternal serum viral RNA load was not significantly different in ZIKV-IG treated animals compared to controls, despite no samples in the 100 mg/kg ZIKV-IG group having detectable virus (Table 26 and Table 27). These patterns are consistent with migration of ZIKV infection from serum to immune privileged tissues over time, and the ability of ZIKV-IG to neutralize virus as demonstrated in previous work (LJI-EB26). Statistical analysis of fetal viral RNA load could not be done due to the complete absence of fetuses in the control group.

TABLE 22
The overall summary statistics of placenta plus decidua viral
RNA load (expressed as log ZIKV genome copies per 18S (log10
ZIKV/18S)) by pre-exposure treatment group at E14.5

| | | | Median | |
|---|---|---|---|---|
| | | Mean (SD) | (log₁₀ ZIKV/ | Min, Max |
| Treatment | Number of | (log₁₀ ZIKV/ | 18S) (25th-75th | (log₁₀ ZIKV/ |
| Group | Fetuses | 18S) | percentile) | 18S) |
| Group A | 42 | 6.478 (0.472) | 6.62 (6.45, 6.73) | 4.42, 7.08 |
| Group B1 | 39 | 5.935 (0.473) | 5.98 (5.79, 6.14) | 4.42, 7.40 |
| Group B2 | 45 | 5.252 (0.713) | 5.45 (5.10, 5.61) | 3.78, 6.81 |

TABLE 23
Two-sample Wilcoxon Rank-Sum Test to compare viral RNA load
in placenta plus decidua of pre-exposure treatment groups

| Treatment Group | p-value compared to Group A^{a} |
|---|---|
| Group B1 | <0.001* |
| Group B2 | <0.001* |

^{a}Wilcoxon Rank-Sum Test with normal approximation.
*= Statistically significant result

TABLE 24
Descriptive statistics of maternal viral RNA
load in spleen at E14.5 by treatment group

| | | | Median | |
|---|---|---|---|---|
| | | Mean (SD) | (log₁₀ ZIKV/ | Min, Max |
| Treatment | Number of | (log₁₀ ZIKV/ | 18S (25th-75th | (log₁₀ ZIKV/ |
| Group | Animals | 18S) | percentile) | 18S) |
| Group A | 6 | 4.235 (0.163) | 4.20 (4.13, 4.26) | 4.09, 4.54 |
| Group B1 | 6 | 3.058 (0.206) | 3.05 (2.97, 3.13) | 2.77, 3.39 |
| Group B2 | 6 | 2.777 (0.678) | 2.83 (2.38, 3.42) | 1.69, 3.51 |

TABLE 25
Descriptive statistics of maternal viral RNA
load in brain at E14.5 by treatment group

| | | | Median | |
|---|---|---|---|---|
| | | Mean (SD) | (log₁₀ ZIKV/ | Min, Max |
| Treatment | Number of | (log₁₀ ZIKV/ | 18S (25th-75th | (log₁₀ ZIKV/ |
| Group | Animals | 18S) | percentile) | 18S) |
| Group A | 6 | 7.635 (0.419) | 7.57 (7.25, 7.89) | 7.21, 8.32 |
| Group B1 | 6 | 6.532 (0.314) | 6.56 (6.25, 6.77) | 6.16, 6.89 |
| Group B2 | 6 | 4.375 (1.650) | 4.47 (3.80, 5.72) | 1.65, 6.15 |

TABLE 26
Descriptive statistics of maternal viral RNA load in
serum (expressed as log₁₀ ZIKV genome copies per milliliters
(ZIKV/mL)) at E14.5 by treatment group

| | | | Median (log₁₀ | |
|---|---|---|---|---|
| | Number | Mean (SD) | ZIKV/mL) | Min, Max |
| Treatment | of | (log₁₀ | (25th-75th | (log₁₀ |
| Group | Animals | ZIKV/mL) | percentile) | ZIKV/mL) |
| Group A | 6 | 3.153 (0.648) | 2.99 (2.59, 3.77) | 2.59, 4.00 |
| Group B1 | 6 | 3.365 (1.450) | 2.59 (2.59, 3.63) | 2.59, 6.20 |
| Group B2 | 6 | 2.590 (0.000) | 2.59 (2.59, 2.59) | 2.59, 2.59 |

TABLE 27
Two-sample Wilcoxon Rank-Sum Test to compare
maternal viral RNA load in spleen at E14.5

| | | p-value compared |
|---|---|---|
| Tissue | Treatment Group | to Group A^{a} |
| Spleen | Group B1 | 0.005* |
| | Group B2 | 0.005* |
| Brain | Group B1 | 0.005* |
| | Group B2 | 0.005* |
| Serum | Group B1 | 0.789 |
| | Group B2 | 0.074 |

^{a}Wilcoxon Rank-Sum Test with normal approximation.
*= Statistically significant result at α = 0.05

Fetal head and body viral load by qRT-PCR were determined where fetuses with discernible heads and bodies were harvested. For those fetuses which had growth restrictions and it was not possible to differentiate the fetal head and body, the entire fetus was considered to be the body. Fetal viral RNA load was not determined for reabsorbed fetuses. As all fetuses harvested from the control group (Group A) were reabsorbed, only descriptive statistics were generated (Table 28 and Table 29). Statistical comparisons for fetal head and body viral load were not conducted.

TABLE 28
Descriptive statistics of fetal body viral RNA load at E14.5 by treatment group

| | | Number | Mean (SD) | | Median (25th-75th | |
|---|---|---|---|---|---|---|
| Treatment | Animal | of | (log₁₀ | | percentile) | Min, Max |
| Group | ID | Fetuses | ZIKV/18S) | | (log₁₀ ZIKV/18S) | (log₁₀ ZIKV/18S) |
| Group B1 | 146 | 1 | 4.230 | (—) | 4.23 (4.23, 4.23) | 4.23, 4.23 |
| Group B2 | 114 | 7 | 3.670 | (0.000) | 3.67 (3.67, 3.67) | 3.67, 3.67 |
| | 139 | 1 | 3.670 | (—) | 3.67 (3.67, 3.67) | 3.67, 3.67 |
| | 143 | 1 | 5.050 | (—) | 5.05 (5.05, 5.05) | 5.05, 5.05 |
| | 146 | 1 | 4.230 | (—) | 4.23 (4.23, 4.23) | 4.23, 4.23 |

TABLE 29
Descriptive statistics of fetal head viral RNA load at E14.5 by treatment group

| | | Number | Mean (SD) | | Median (25th-75th | |
|---|---|---|---|---|---|---|
| Treatment | Animal | of | (log₁₀ | | percentile) | Min, Max |
| Group | ID | Fetuses | ZIKV/18S) | | (log₁₀ ZIKV/18S) | (log₁₀ ZIKV/18S) |
| Group B1 | 146 | 1 | 4.630 | (—) | 4.63 (4.63, 4.63) | 4.63, 4.63 |
| Group B2 | 114 | 7 | 3.620 | (0.000) | 3.62 (3.62, 3.62) | 3.62, 3.62 |
| | 137 | 1 | 5.050 | (—) | 5.05 (5.05, 5.05) | 5.05, 5.05 |
| | 139 | 2 | 3.770 | (0.212) | 3.77 (3.62, 3.92) | 3.62, 3.92 |

### Example 7: Neutralization Potency and Antibody Dependent Enhancement of Dengue Virus Type 2 and Dengue Virus Type 3 by ZIKV-IG

The neutralization potency and antibody dependent enhancement (ADE) of Dengue Virus Type 2 and Dengue Virus Type 3 by ZIKV-IG was assessed in the following study.

### Focus Forming Reduction Neutralization Test (FRNT₅—Vero Cell Line

Focus forming reduction neutralization (FRNT) assays were performed in a similar manner to those described in Sukupolvi-Petty et al., J. Virol., 87:8826-8842, 2013 and were used to determine the 50% effective neutralizing concentration (FRNT₅₀ or EC₅₀ of immunoglobulins against Zika virus (ZIKV) PRVABC59, Dengue virus type 2 (DENV2), and Dengue virus type 3 (DENV3). Two-fold serial dilutions of ZIKV-IG antibody (2.5 mg/mL to 19 ng/mL) were mixed with ˜80 focus forming units (FFU) of virus, incubated at 37° C. for 1 hour, then added to Vero cell monolayers in 96 well plates for 1 hour at 37° C. to allow virus adsorption. Non-specific Ig antibody was used as a negative control. Cells were then overlaid with 1% methylcellulose and incubated for two days. Monolayers were fixed for 1 hour at room temperature, washed and permeabilized. Infected cell foci were stained by incubating cells with 500 ng/ml of flavivirus cross-reactive MAb 4G2 for 1 hour at 4° C., washed and detected by incubating cells with a 1:5,000 dilution of horseradish peroxidase conjugated goat anti-mouse IgG for 1 hour. After washing, staining was visualized by addition of TrueBlue detection reagent. Infected foci were enumerated using a CTL-Immunospot S6 (Cellular Technology Limited).

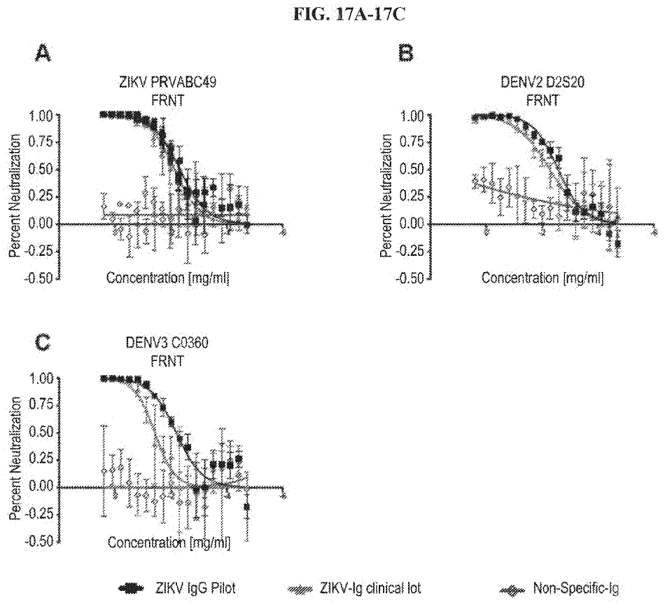

To evaluate neutralization potential, different concentrations of immune globulin were incubated with either ZIKV, DENV2 or DENV3 for 1 hour at 37° C. then incubated on Vero cells. The FRNT₅₀ value for ZIKV-IG pilot (potency titer of 1:18480) and ZIKV-IG clinical lot (potency titer of 1:11673) for ZIKV PRVABC59 is 4.8 μg/mL and 7.4 μg/mL respectively (FIG. 17A and Table 30). The FRNT₅₀ value for ZIKV-IG pilot and ZIKV-IG clinical lot for DENV2 is 2.5 μg/mL and 5.2 μg/mL respectively (FIG. 17B and Table 30). The FRNT₅₀ value for ZIKV-IG pilot and ZIKV-IG clinical lot for DENV3 is 6.4 μg/mL and 38.3 μg/mL respectively (FIG. 17C and Table 30).

TABLE 30
Half maximum effective concentration of the two ZIKV-IG lots.

| | ZIKV-IG pilot lot | ZIKV-IG clinical lot |
|---|---|---|
| | EC₅₀ mg/mL | EC₅₀ mg/mL |
| ZIKV | 0.0048 | 0.0074 |
| DENV2 | 0.0025 | 0.0052 |
| DENV3 | 0.0064 | 0.0383 |

### Antibody Dependent Enhancement—K562 Cell Line

Antibody dependent enhancement assays (ADE) are performed and analyzed using procedures described in Bardina et al., Science, 356(6634):175-180, 2017. ADE assays are used to quantify the enhancement potential of immunoglobulin specific for ZIKV in comparison to two immunoglobulin controls (GAMUNEX and GAMMAGARD) and a non-Zika specific immunoglobulin control (Isotype Control). Serial 4-fold dilutions of antibody (2.5 mg/mL to 2.4 ng/mL) are mixed with ZIKV multiplicity of infection (MOI) of 1 and then incubated at 37° C. for 1 hour to allow the formation of immune complexes. Immune complexes are added to K562 cells that express the Fc-gamma receptor CD32A and incubated for 2-3 days. Cells are fixed, washed, permeabilized and stained with flavivirus cross-reactive MAb 4G2. Cells are stained with A647 conjugated goat anti-mouse IgG secondary antibody, washed and analyzed on an Attune flow cytometer.

### Antibody Dependent Enhancement—In Vivo Assessment in Immunocompromised Mice

The in vitro work described above was expanded upon in vivo using Ifnar1^{-/-} immunocompromised mice using the pilot lot of ZIKV-IG. Animals were treated with either 0.05 mg ZIKV-IG/animal, or 2 mg Gamunex/animal (a hyperimmune IVIG control), or 0.02 mg monoclonal antibody 4G2/animal (a pan-flavivirus reactive antibody control), or 0.02 mg IgG2a/animal (monoclonal antibody isotype control) 24 hours prior to infection with either 1×10⁴ FFU Dengue virus serotype 2 (DENV2) strain D2S20 or 1×10⁷ FFU Dengue virus serotype 3 (DENV3) strain C0360/94. Animals were monitored for survival, body weights and clinical scores for 21 days.

### In Vivo Assessment Results—DENV2.

All mice, independent of treatment, showed signs of DENV2 infection, including weight loss and clinical signs of disease. Mortality rates (Table 31), weight loss (Table 32) and severity of clinical signs of disease (Table 33) were consistently significantly greater in ZIKV-IG treated animals than isotype (polyclonal antibody) negative controls and were similar to those observed in 4G2 (positive) controls. There were no significant differences between treatment with 4G2 and ZIKV-IG.

TABLE 31
Summary Statistics of Mice Surviving to Day 21 When
Infected with DENV2 After ZIKV-IG Treatment

| | | Proportion | | |
|---|---|---|---|---|
| | | of Mice | | |
| | | Survived | | |
| | | to Day 21 | | |
| | Number | (95% Exact | | Bonferroni |
| | of Mice | Binomial | Fisher's Exact | Adjusted |
| Treatment | Survived | Confidence | Test p-value | p-value vs |
| Group | to Day 21 | Interval) | vs 4G2 Group | 4G2 Group |
| 4G2 | 3/10 | 0.30 (0.07, 0.65) | | |
| Gamunex | 9/10 | 0.90 (0.55, 1.00) | 0.02 | 0.06 |
| Isotype | 10/10 | 1.00 (0.69, 1.00) | 0.003 | 0.01* |
| ZIKV-IG | 5/10 | 0.50 (0.19, 0.81) | 0.65 | 1 |

*= Statistically significant result.

TABLE 32
Analysis of Changes in Body Weight at Day 5 Relative to
Day 0 In Mice Infected with DENV2 After ZIKV-IG Treatment

| Pairwise Comparison | p-value^{a} |
|---|---|
| 4G2 vs. Gamunex | 0.0022* |
| 4G2 vs. Isotype | 0.0022* |
| 4G2 vs. ZIKV-IG | 0.8395 |
| Gamunex vs. Isotype | 0.5242 |
| Gamunex vs. ZIKV-IG | 0.0105* |
| Isotype vs. ZIKV-IG | 0.0063* |

^{a}DSCF adjusted p-value for multiple comparisons
*= Statistically significant result

TABLE 33
Kruskal-Wallis Test for Clinical Score at Day 5 In
Mice Infected with DENV2 After ZIKV-IG Treatment

| Pairwise Comparison | p-value^{a} |
|---|---|
| 4G2 vs. Gamunex | 0.0075* |
| 4G2 vs. Isotype | 0.0005* |
| 4G2 vs. ZIKV-IG | 0.4662 |
| Gamunex vs. Isotype | 0.6958 |
| Gamunex vs. ZIKV-IG | 0.1258 |
| Isotype vs. ZIKV-IG | 0.0116* |

^{a}DSCF adjusted p-value for multiple comparisons
*= Statistically significant result

### In Vivo Assessment Results—DENV3

All Ifnar1−/− mice, independent of treatment, showed signs of DENV3 infection as assessed by weight loss. Administration of a sub-neutralizing dose of ZIKV-IG (50 μg) to Ifnar1−/− mice one day prior to a sub-lethal infection with DENV3 resulted in a significantly lower body average weight compared to the isotype (negative) control on day 4 post infection and was not significantly different from the 4G2 (positive) control group (Table 35). Enhanced mortality was not observed in the ZIKV-IG treated group as only one of 18 mice succumbed to infection following ZIKV-IG treatment compared to 11 of 16 mice for the 4G2 treated control group (Table 34). No significant differences in clinical scores were observed (Table 36). This suggests that ZIKV-IG treatment contributes to the enhancement of morbidity but not mortality associated with ADE during a DENV3 infection.

TABLE 34
Summary Statistics of Mice Surviving to Day 21 When
Infected with DENV3 After ZIKV-IG Treatment

| | | Proportion | | |
|---|---|---|---|---|
| | | of Mice | | |
| | | Survived | | |
| | | to Day 21 | | |
| | Number | (95% Exact | | Bonferroni |
| | of Mice | Binomial | Fisher's Exact | Adjusted |
| Treatment | Survived | Confidence | Test p-value | p-value vs |
| Group | to Day 21 | Interval) | vs 4G2 Group | 4G2 Group |
| 4G2 | 5/16 | 0.31 (0.11, 0.59) | | |
| Gamunex | 4/17 | 0.24 (0.07, 0.50) | 0.708 | 1 |
| Isotype | 19/19 | 1.00 (0.82, 1.00) | <0.001* | <0.001* |
| ZIKV-IG | 17/18 | 0.94 (0.73, 1.00) | <0.001* | <0.001* |

*= Statistically significant result.

TABLE 35
Analysis of change in body weight at day 4 relative to day
0 in mice Infected with DENV3 After ZIKV-IG Treatment

| Pairwise Comparison | p-value^{a} |
|---|---|
| 4G2 vs. Gamunex | 0.688 |
| 4G2 vs. Isotype | 0.001* |
| 4G2 vs. ZIKV-IG | 0.069 |
| Gamunex vs. Isotype | 0.016* |
| Gamunex vs. ZIKV-IG | 0.165 |
| Isotype vs. ZIKV-IG | <0.001* |

^{a}DSCF adjusted p-value for multiple comparisons.
*= Statistically significant result.

TABLE 36
Kruskal-Wallis test for clinical score at day 4 in
mice Infected with DENV3 After ZIKV-IG Treatment

| Pairwise Comparison | p-value |
|---|---|
| 4G2 vs. Gamunex | 1.0000 |
| 4G2 vs. Isotype | 1.0000 |
| 4G2 vs. ZIKV-IG | 1.0000 |
| Gamunex vs. Isotype | 1.0000 |
| Gamunex vs. ZIKV-IG | 1.0000 |
| Isotype vs. ZIKV-IG | 1.0000 |

### Conclusion

Both the pilot lot and clinical lot of ZIKV-IG exhibited in vitro neutralization potency of ZIKV (PRVABC59), Dengue Type 2 virus, and Dengue Type 3 virus strains. Neutralization potency of the ZIKV-IG lots to Dengue Type 2 and Dengue Type 3 virus strains is at similar levels to that observed against ZIKV.

The pilot lot of ZIKV-IG exhibited characteristics of ADE when administered at a sub-neutralizing dose 24 hours before infection of Ifnar−/− mice with DENV2 or DENV3, including mortality, morbidity (weight loss) and clinical symptoms of disease.

### Example 8: Antibody Dependent Enhancement (ADE) of Zika Infection by ZIKV-IG in Ifnar−/− Mice

Study

As explained in Example 7, two lots of ZIKV-IG were tested for neutralization and will be tested for enhancement in Vero and human K562 cell lines. Neutralization assay results indicated that ZIKV-IG was highly potent and had an IC₅₀ value between 4.8 and 7.4 μg/mL.

To determine the relationship between disease outcome in vivo and the amount of antibody administered for ADE assessment, various dose levels of ZIKV-IG will be tested in Ifnar1−/− mice in efficacy studies in a post-exposure prophylaxis setting. Dose levels ranging from 0.1 mg/kg to 50 mg/kg of ZIKV-IG were used in a survival study and 0.5-50 mg/kg were used in a viral load analysis study, using the methodology described in the other examples. For the ADE analysis, endpoints including the median time to death (MTD), clinical severity, body weight change and viral load in target tissues will be considered, using the methodology described in the other examples.

### Results

When there is ADE enhancement of Zika infection, a faster death of antibody-treated animals is anticipated compared to control treated animals (PBS treated). This can be measured by MTD analysis.

The clinical severity of disease at various critical times during the study period was expressed as mean, median and range values for comparison (FIG. 6). Clinical score data was analyzed using the Jonckheere-Terpstra test to determine if there is a statistically significant trend between an ordinal independent variable (dose) and an ordinal dependent variable (clinical score). All clinical scores recorded for each animal in each group were used in this analysis, without regard to their timing. The ordinal analysis suggests that the clinical scores were linearly higher (indicating the higher severity of the clinical disease) in animals given lower doses of ZIKV-IG, despite attrition by death or euthanasia. In addition, there was a statistically significant difference in clinical scores between the control treated group versus the 50 mg/kg-treated group (Bonferroni Adjusted p<0.0001) and the control treated group versus the 10 mg/kg-treated group (Bonferroni Adjusted p=0.0286) with respect to severity of the disease. There was no statistically significant difference in clinical scores between the lower dose-treated groups (2.0-0.1 mg/kg) and the control treated group.

Moreover, mice treated with ZIKV-IG at lower doses (2 and 0.5 mg/kg) had a significantly higher liver viral RNA load (Table 37) by quantitative reverse transcriptase-polymerase chain reaction (qRT-PCR) analysis when compared to the control group on day 3 and 7 (p=0.009 and p=0.017, respectively). There was no significant difference between lower dose groups and the control for the other tissues tested.

TABLE 37
Viral load by qRT-PCR (log ZIKV copies/18S mean +
SD) viral load in key tissues- for low dose groups.

| | ZIKV-IG | | |
|---|---|---|---|
| Tissue | Treatment Dose | Day 3 Viral Load | Day 7 Viral Load |
| Brain | PBS | 4.30 (0.26) | 7.82 (0.24) |
| | 2.0 mg/kg | 3.77 (0.37) | 7.56 (0.24) |
| | 0.5 mg/kg | 4.24 (0.51) | 7.62 (0.23) |
| Serum | PBS | 8.63 (0.39) | 5.28 (0.45) |
| | 2.0 mg/kg | 8.36 (0.55) | 5.61 (0.33) |
| | 0.5 mg/kg | 8.84 (0.37) | 5.17 (0.65) |
| Testes | PBS | 5.17 (0.16) | 6.42 (0.50) |
| | 2.0 mg/kg | 5.82 (0.16) | 6.36 (0.16) |
| | 0.5 mg/kg | 5.77 (0.45) | 6.50 (0.37) |
| Ovaries | PBS | 7.52 (0.09) | 5.84 (0.56) |
| | 2.0 mg/kg | 7.14 (0.17) | 5.95 (0.30) |
| | 0.5 mg/kg | 7.40 (0.29) | 5.91 (0.16) |
| Sciatic | PBS | 6.12 (0.85) | 7.43 (0.71) |
| nerve | 2.0 mg/kg | 6.28 (0.90) | 6.65 (0.26) |
| | 0.5 mg/kg | 6.52 (0.48) | 7.17 (0.41) |
| Liver | PBS | 5.15 (0.25) | 3.87 (0.71) |
| | 2.0 mg/kg | 5.70 (0.75) | 5.60 (0.18) * |
| | 0.5 mg/kg | 5.88 (0.25) * | 5.00 (0.45) * |
| Spleen | PBS | 7.30 (0.17) | 5.70 (0.18) |
| | 2.0 mg/kg | 7.41 (0.19) | 4.98 (0.45) |
| | 0.5 mg/kg | 7.34 (0.10) | 5.56 (0.08) |
| Kidney | PBS | 5.67 (0.39) | 4.68 (0.31) |
| | 2.0 mg/kg | 5.56 (0.33) | 5.00 (0.27) |
| | 0.5 mg/kg | 5.83 (0.27) | 5.47 (0.57) |

* Significant difference compared to control.

These results were confirmed by focus form assay (FFA) analysis using the methodology described in the other examples. Results from the FFA analysis are shown in Table 38.

TABLE 38
Viral load by FFU ((log ZIKV/mL mean + SD))
viral load in key tissues for lower dose groups.

| | ZIKV-IG | | |
|---|---|---|---|
| Tissue | Treatment Dose | Day 3 Viral Load* | Day 7 Viral Load* |
| Brain | PBS | 2.03 (0.44) | 5.86 (0.20) |
| | 2.0 mg/kg | 1.62 (0.25) | 4.76 (0.85) |
| | 0.5 mg/kg | 1.87 (0.27) | 5.55 (0.56) |
| Serum | PBS | 3.87 (0.46) | 0.70 (0.00) |
| | 2.0 mg/kg | 3.49 (0.48) | 0.70 (0.00) |
| | 0.5 mg/kg | 4.16 (0.42) | 0.70 (0.00) |
| Testes | PBS | 2.83 (0.57) | 6.03 (1.00) |
| | 2.0 mg/kg | 3.58 (0.54) | 6.35 (0.63) |
| | 0.5 mg/kg | 3.05 (0.78) | 6.16 (0.23) |
| Ovaries | PBS | 2.95 (0.42) | 1.51 (0.76) |
| | 2.0 mg/kg | 2.61 (0.19) | 0.80 (0.17) |
| | 0.5 mg/kg | 2.69 (0.45) | 0.90 (0.17) |
| Sciatic | PBS | 1.08 (0.40) | 2.25 (0.46) |
| nerve | 2.0 mg/kg | 0.86 (0.39) | 1.37 (0.76) |
| | 0.5 mg/kg | 0.90 (0.25) | 2.00 (0.24) |
| Liver | PBS | 3.52 (0.23) | 1.71 (0.58) |
| | 2.0 mg/kg | 3.42 (0.14) | 1.50 (0.41) |
| | 0.5 mg/kg | 3.68 (0.28) | 1.91 (0.52) |
| Spleen | PBS | 5.65 (1.43) | 4.22 (0.12) |
| | 2.0 mg/kg | 6.19 (0.14) | 3.18 (0.59) |
| | 0.5 mg/kg | 6.23 (0.24) | 3.99 (0.21) |
| Kidney | PBS | 4.43 (0.47) | 2.29 (0.61) |
| | 2.0 mg/kg | 4.27 (0.15) | 2.07 (0.49) |
| | 0.5 mg/kg | 4.75 (0.22) | 2.16 (0.77) |

*Serum, sciatic nerve and ovaries are expressed as FFU/mL of tissue, all other tissues are expressed as FFU/g tissue.

Unlike the qRT-PCR analysis results, the liver from lower dose groups (2 mgand 0.5 mg/kg) did not show any significant (p=1.0) increase in viral load compared to the control group. The virus titer results in all of the tissues tested from low dose groups were similar to control groups.

### Antibody Dependent Enhancement—In Vivo Assessment in Immunocompromised Mice in a Pre-Exposure Prophylaxis Setting

The in vitro and in vivo work described above was expanded upon in vivo using Ifnar1^{-/-} immunocompromised mice using the pilot lot of ZKV-IG administered prior to infection. Animals were treated with either 0.05 mg ZIKV-IG/animal, or 2 mg Gamunex/animal (a hyperimmune IVIG control), or 0.02 mg monoclonal antibody 4G2/animal (a pan-flavivirus reactive antibody control), or 0.02 mg IgG2a/animal (monoclonal antibody isotype control) 24 hours prior to infection with 1×10⁵ FFU Zika (ZIKV) strain PRVABC59. Animals were monitored for survival, body weights and clinical scores for 21 days.

In a follow-on study Ifnar1^{-/-} immunocompromised mice were administered either 0.5 mg, 0.05 mg or 0.01 mg of the pilot lot of ZIKV-IG per animal 24 hours prior to infection with ZIKV strain PRVABC59. Animals were sacrificed at either Day 3 or Day 7 and selected tissues (brain, kidney, spleen, liver) harvested and assayed for live virus by focus-forming assay (FFA) and viral RNA by quantitative reverse transcriptase PCR (qRT-PCR). Blood samples were also collected at time of sacrifice for processing into serum and plasma. Plasma was used for analysis of viral RNA by qRT-PCR and hematological parameters while serum was used for assay of live virus by FFA analysis and assay of clinical chemistry parameters.

### In Vivo Assessment Results—ZIKV

In the initial study all mice independent of treatment lost weight, with peak weight loss occurring three days post infection. This weight loss was indicative of infection. Mice that received ZIKV-IG exhibited a statistically significant decrease in body weight on study days 7 and 14 following ZIKV infection based upon mean body weight change from baseline compared to Isotype control (Table 39). This increase in weight loss appeared to be more pronounced male mice compared to females. No statistically significant differences in clinical scores were observed between the ZIKV-IG treated group and the Isotype controls (Table 40). No animals from any study group succumbed to infection.

In the follow-on study the 0.05 mg/animal ZIKV-IG dose significantly increased live virus titers over that of PBS controls at Day 3 in serum and kidney, while the 0.01 mg/animal ZIKV-IG dose significantly increased live virus titers in serum at Day 7 (Table 41). The 0.01 mg/animal ZIKV-IG dose significantly increased viral RNA levels in brain at Day 3 and kidney and liver at Day 7. The 0.05 mg/animal ZIKV-IG dose also significantly increased viral RNA in liver at Day 7. In contrast, the 0.05 mg/animal ZIKV-IG dose significantly reduced viral RNA levels in both kidney and serum at Day 3 (Table 42).

TABLE 39
Analysis of Mean Body Weight Change from Baseline
to Study Day by Treatment Group and Sex

| | | | | Dunnett's Test |
|---|---|---|---|---|
| | | | Difference | Adjusted p-value |
| | | | Between | vs Isotype |
| Study Day | Comparison | Sex | Means | (* = significant) |
| Day 7 | 4G2 - | Female | −0.105 | 0.660 |
| | ISOTYPE | Male | −0.102 | 0.661 |
| | | Overall | −0.122 | 0.603 |
| | GAMUNEX - | Female | 0.272 | 0.914 |
| | ISOTYPE | Male | 0.319 | 0.918 |
| | | Overall | 0.259 | 0.936 |
| | ZIKVIG - | Female | −0.603 | 0.200 |
| | ISOTYPE | Male | −0.809 | 0.118 |
| | | Overall | −0.725 | 0.047* |
| Day 14 | 4G2 - | Female | 0.114 | 0.870 |
| | ISOTYPE | Male | 0.021 | 0.763 |
| | | Overall | 0.064 | 0.826 |
| | GAMUNEX - | Female | 0.285 | 0.961 |
| | ISOTYPE | Male | 0.268 | 0.929 |
| | | Overall | 0.269 | 0.964 |
| | ZIKVIG - | Female | −0.196 | 0.469 |
| | ISOTYPE | Male | −1.700 | 0.000* |
| | | Overall | −0.951 | 0.001* |
| Day 21 | 4G2 - | Female | 0.124 | 0.905 |
| | ISOTYPE | Male | 0.497 | 0.969 |
| | | Overall | 0.304 | 0.974 |
| | GAMUNEX - | Female | 0.396 | 0.995 |
| | ISOTYPE | Male | 0.052 | 0.782 |
| | | Overall | 0.202 | 0.934 |
| | ZIKVIG - | Female | 0.150 | 0.925 |
| | ISOTYPE | Male | −0.832 | 0.090 |
| | | Overall | −0.347 | 0.219 |

TABLE 40
Kruskal-Wallis Analysis for Differences in Median Clinical
Score Between Study Groups on Selected Study Days in
Mice Infected with ZIKV After ZIKV-IG Treatment

| Study Day | p-value |
|---|---|
| Day 7 | 0.9202 |
| Day 14 | 0.5092 |
| Day 21 | 1.000 |

TABLE 41
Analysis of Live Virus in Tissues of Animals
Treated with ZIKV-IG Compared to PBS Controls

| | |
|---|---|
| | Bonferroni-adjusted Wilcoxon |
| | rank sum p-value vs. |
| Treatment | PBS controls |

| Tissue | Group | Day 3 | Day 7 |
|---|---|---|---|
| Brain | 0.50 mg ZIKV-IG | 0.226 | 1 |
| | 0.05 mg ZIKV-IG | 0.374 | 0.517 |
| | 0.01 mg ZIKV-IG | 0.547 | 0.158 |
| Kidney | 0.50 mg ZIKV-IG | 0.092 | 1 |
| | 0.05 mg ZIKV-IG | 0.022** | 1 |
| | 0.01 mg ZIKV-IG | 0.030** | 1 |
| Liver | 0.50 mg ZIKV-IG | 1 | 1 |
| | 0.05 mg ZIKV-IG | 1 | 1 |
| | 0.01 mg ZIKV-IG | 1 | 1 |
| Serum | 0.50 mg ZIKV-IG | 0.106 | 0.703 |
| | 0.05 mg ZIKV-IG | 0.004** | 0.703 |
| | 0.01 mg ZIKV-IG | 0.715 | 0.015** |
| Spleen | 0.50 mg ZIKV-IG | 1 | 0.922 |
| | 0.05 mg ZIKV-IG | 1 | 0.819 |
| | 0.01 mg ZIKV-IG | 1 | 0.07 |

**= Significant Wilcoxon rank sum p-value (<= 0.05)
Arrows indicate if ZIKV-IG treatment groups has elevated or reduced levels of live virus compared to PBS controls.

TABLE 42
Analysis of Viral RNA in Tissues of Animals
Treated with ZIKV-IG Compared to PBS Controls

| | |
|---|---|
| | Bonferroni-adjusted Wilcoxon |
| | rank sum p-value vs. |
| Treatment | PBS controls |

| Tissue | Group | Day 3 | Day 7 |
|---|---|---|---|
| Brain | 0.50 mg ZIKV-IG | 0.267 | 0.113 |
| | 0.05 mg ZIKV-IG | 0.558 | 0.312 |
| | 0.01 mg ZIKV-IG | 0.003** | 0.052 |
| Kidney | 0.50 mg ZIKV-IG | 0.034** | 1 |
| | 0.05 mg ZIKV-IG | 0.637 | 0.227 |
| | 0 01 mg ZIKV-IG | 0.135 | 0.004** |
| Liver | 0.50 mg ZIKV-IG | 0.312 | 0.052 |
| | 0.05 mg ZIKV-IG | 0.094 | 0.034** |
| | 0.01 mg ZIKV-IG | 1 | 0.014** |
| Serum | 0.50 mg ZIKV-IG | 0.038** | 0.192 |
| | 0.05 mg ZIKV-IG | 0.267 | 0.113 |
| | 0.01 mg ZIKV-IG | 0.558 | 0.312 |
| Spleen | 0.50 mg ZIKV-IG | 0.003** | 0.052 |
| | 0.05 mg ZIKV-IG | 0.034** | 1 |
| | 0.01 mg ZIKV-IG | 0.637 | 0.227 |

**= Significant Wilcoxon rank sum p-value (<= 0.05)
Arrows indicate if ZIKV-IG treatment groups has elevated or reduced levels of live virus compared to PBS controls.

### Conclusion

Based on the results of these studies we can conclude that, while Ifnar1−/− mice exhibit signs of ZIKV associated infection independent of treatment, administration of a sub-neutralizing dose of ZIKV-IG (0.050 mg/animal) 24 hours prior to a sub-lethal infection contributes to increased levels of viral infection and disease-associated morbidity which are indicative of ADE.

### Example 9: Evaluation of ZIKV-IG in Pregnant Rhesus Macaques Exposed to Zika Virus

To evaluate the efficacy of ZIKV-IG in pregnant non-human primates infected with ZIKV in a post-exposure prophylaxis (PEP) setting and to determine pharmacokinetic (PK) profiles of ZIKV-IG in pregnant monkeys, the following study was performed. More specifically, this study evaluated ZIKV-IG for neutralization of virus in pregnant rhesus macaques infected with ZIKV around 41-49 days of gestation (the end of the first trimester and the beginning of the second trimester). The endpoint measures include viral load in the mother and fetus and histopathological evaluation of fetuses. The outcome measures include a reduction of infection in mothers, prevention of viral persistence, prevention of transfer of infection to the fetus and fetal growth in treated groups in comparison to control. Pharmacokinetic analysis was also conducted to calculate PK parameters including AUC₀₋ₜ, AUC_{0-inf}, Cₘₐₓ, Tₘₐₓ, Kₑₗ, t_{1/2}, Cl and V_{d}.

### Animals, Treatments, and Sample Collection

Eight naïve pregnant female rhesus macaques between 5-9 kg were obtained from a pathogen-free colony at the Wisconsin National Primate Research Center (WNPRC, University of Wisconsin-Madison). All macaques were free of Macacine herpesvirus 1, simian retrovirus type D (SRV), simian T-lymphotropic virus type 1 (STLV) and simian immunodeficiency virus and had no prior exposure history of flaviviruses or flavivirus-based vaccines. Animals were in good health, free of malformations and exhibited no signs of any clinical disease and were randomized into one of two treatment groups. The macaques were subcutaneously (SC) exposed to 1.0×10⁴ plaque-forming units (PFU) of Zika virus (ZIKV; Strain PRVABC59; GenBank: KU501215) and intravenously treated with a dose of ZIKV-IG or placebo control (50 mg/kg for both) at 24 hours post-infection and re-dosed at 5 days post-infection. Human immunoglobulin with a low anti-ZIKV E protein titer was used as the placebo control and was administrated to the control group at the designated time points after infection.

Animals were infected with virus on day 45±4 of gestation via subcutaneous (SC) route inoculation as shown in Table 43.

TABLE 43
Study Design

| | | | Dose Level | | Dose Level | |
|---|---|---|---|---|---|---|
| Group | Number of | Challenge | (PFU) | | (total protein | Observations and |
| Number | monkeys | Material | and route | Treatment | mg/kg) and route | Tests |
| 1 | 4 | ZIKV | 10⁴, SC | ZIKV-IG | ~50, IV | Everyday clinical |
| 2 | 4 | ZIKV | 10⁴, SC | Placebo | ~50, IV | signs |
| | | | | | | Body weight, |
| | | | | | | Temperatures |
| | | | | | | Ultrasound for |
| | | | | | | development |
| | | | | | | Plasma viremia |
| | | | | | | PK |
| | | | | | | Tissue viral load |
| | | | | | | and histopathology |

SC: subcutaneous; IV: intravenous

Following infection, each group was intravenously (IV) administered either 50 mg/kg ZIKV-IG or placebo IG at 24 (+/−2) hours and re-dosed at 5 days post-infection. Blood was collected at designated times before and after infusion for serum ZIKV-IG pharmacokinetic (PK) and plasma viral load tests. Following the collection of blood and serum separation, serum aliquots of at least four vials per sample were stored at −80° C. ZIKV-IG titer was determined in serum samples by ELISA. Viral load in plasma and tissue samples was determined by qRT-PCR and plaque assay (Plaque Forming Unit, PFU). Clinical signs, body weight, and body temperature were recorded frequently. Fetus development was monitored by ultrasound once a week and heartbeat by ultrasound twice a week. Cesarean section was performed on gestational day 155±4, and fetectomy for tissue viral load quantification and histopathology was performed.

Following infection, clinical signs were assessed. The schedule of sample collection and clinical observations is shown in Table 44.

TABLE 44
Sampling days/analysis/outcome measures.

| Schedule | Specimen Collection | Analyses |
|---|---|---|
| Pre-exposure | Blood (plasma and serum) | Serum ZIKV-IG, baseline for PK; |
| (~day −8 and/or −1) | | Clinical sign baseline/plasma viremia |
| | | baseline (qRT-PCR/PFU)/bodyweight, |
| | | temperature baseline |
| Infection (Day 0) | Blood (plasma and serum) | Additional baseline samples stored for |
| | | future use in assays |
| Treatment (Day 1) | Blood (plasma and serum) at day | Plasma viremia, serum ZIKV-IG |
| | 1, serum only 1 and 6 hours post | (ELISA) and PRNT for PK |
| | IgG infection. | |
| Treatment (Day 5) | Blood (plasma and serum) at day | Plasma viremia, serum ZIKV-IG |
| | 5, serum only 1 and 6 hours post | (ELISA) and PRNT for PK |
| | IgG infection. | |
| Post-infection | Blood (plasma, serum), collection | Plasma viremia, serum ZIKV-IG |
| | as indicated in Section 8.4, 8.5 | (ELISA) and PRNT for PK |
| | (blood collection days/volume are | |
| | limited due to max volume | |
| | limitations) | |
| Terminal (Day 110 | Blood (plasma and serum) | Final bleed for all analysis: plasma |
| post infection, Day | | viremia, serum antibody concentration |
| 155 of gestation) | | (ELISA, PRNT) |
| | Necropsy for tissue collection | Maternal/fetal interface and ~60 fetal |
| | (see Section 8.5.3) | tissues for persistent of virus analysis |
| | | (see 8.5.3); |
| | | Histopathology of both maternal/fetal |
| | | interface and fetal tissues; |

PRNT = plaque reduction neutralization test;
ELISA = enzyme-linked immunosorbent assay

As shown in Table 44, blood was collected from all animals prior to challenge (baseline, Day-8 and −1), infection day (day −1 relative to first human immunoglobulin (HIG) injection), 0 hours (prior to first HIG injection), 1, 6, 24, and 48, and 72 hours (+/−2 hours) and on 4 (0, 1, and 6 hours post-second-injection), 5, 6, 8, 12, 15, 19, 22, 26, 29, 33, 36, 40, 43, days post-first-injection, twice/week until maternal viremia is undetectable at two consecutive time points, and then once/week until the termination of pregnancy. Plasma was used to measure Zika viral load at each time point and serum was used in PRNT assays and PK analysis using whole virion binding ELISA assays. Caesarean section was performed on day 110 (±4) post-infection (day 155 (±4) of gestation) and maternal and fetal blood samples were collected for viral titer and anti-ZIKV antibody titer. In addition, tissues samples were obtained from fetuses and processed for viral titer (qRT-PCR) and histopathology analysis.

### Viral Load Analysis

Viral load assays were performed on plasma samples collected at each time point. Briefly, vRNA was isolated from plasma samples and quantified by a one-step qRT-PCR using Zika virus-specific primers and a probe on a LightCycler 480. Replication competent virus was quantified by plaque assay by adding serum serial dilutions onto Vero cell cultures that were then overlaid with oxoid agar and stained upon plaque formation. Plaques were counted each day until no significant increase in plaques are observed.

For viral load analyses from either qRT-PCR, genome copies mg or mL of tissue (respectively) at each time point was summarized and compared between control and ZIKV-IG treated groups using the descriptive statistics.

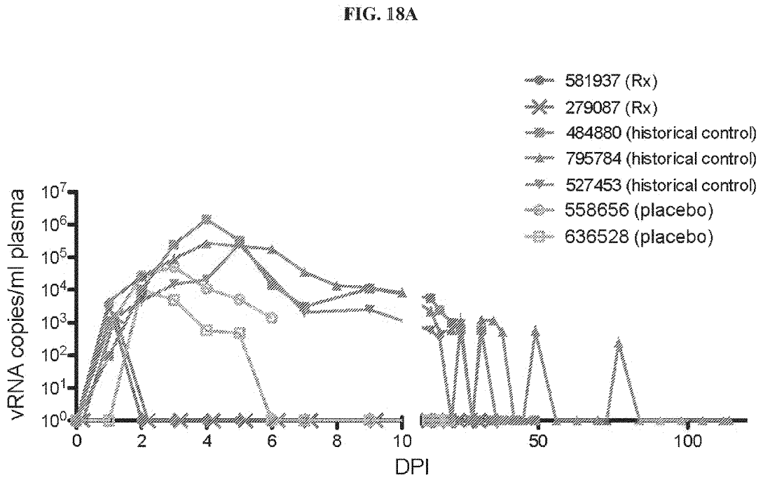

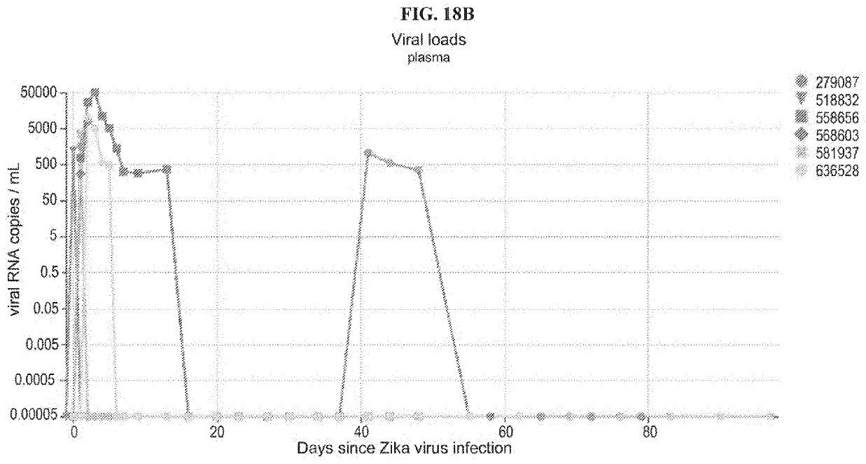

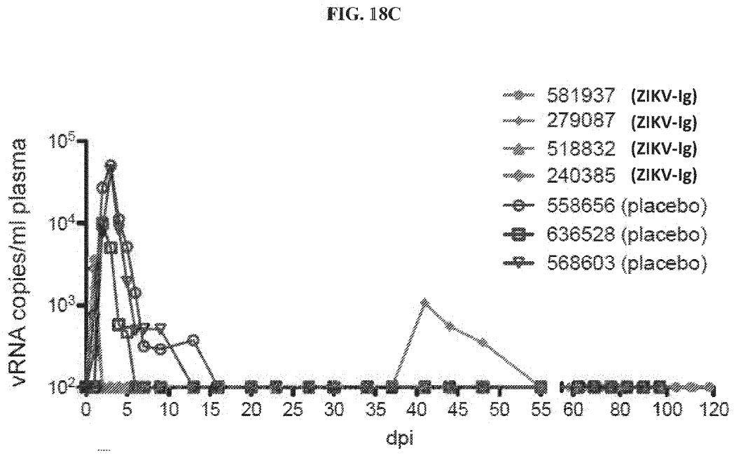

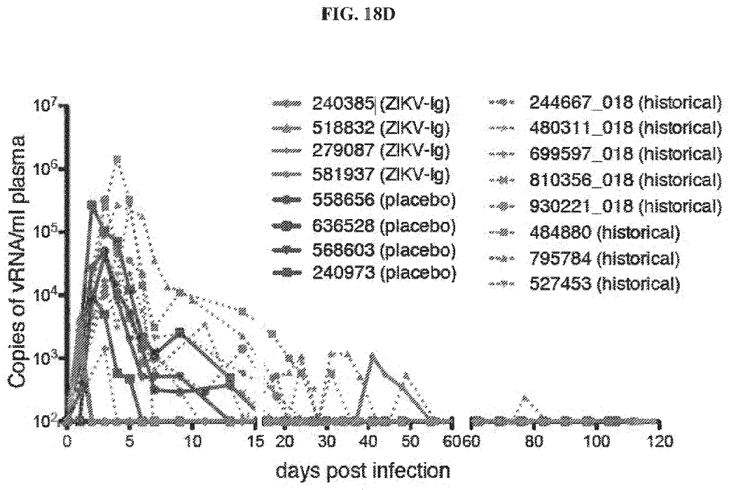

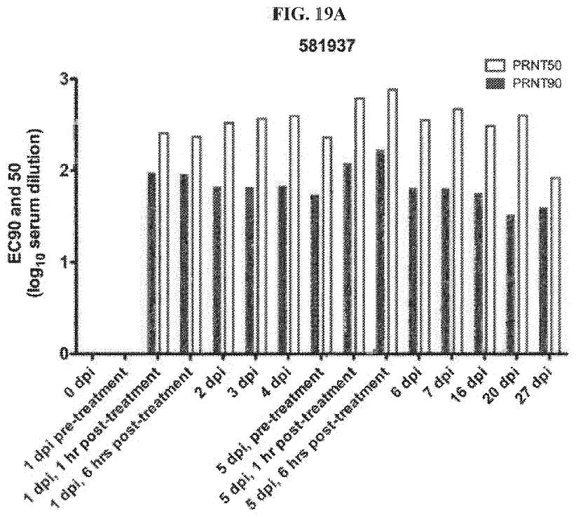

As shown in FIG. 18A, FIG. 18B, FIG. 18C, and FIG. 18D, viral loads in the serum of ZIKV-IG treated groups (ZIKV-Ig) were significantly decreased over time compared to the viral loads of historical controls and placebo groups. FIG. 18C and FIG. 18D each show elimination of plasma viremia by day 2 post infection for ZIKV-IG treated group animals (581937, 279087, 518832, and 240385) FIG. 18D also shows that the placebo groups (558656, 636528, 568603, and 240973) exhibit overall shorter duration of viremia than historical controls; however, ZIKV-IG treated animals had a lower viral load and shorter duration of viremia compared to both placebo and historical controls.

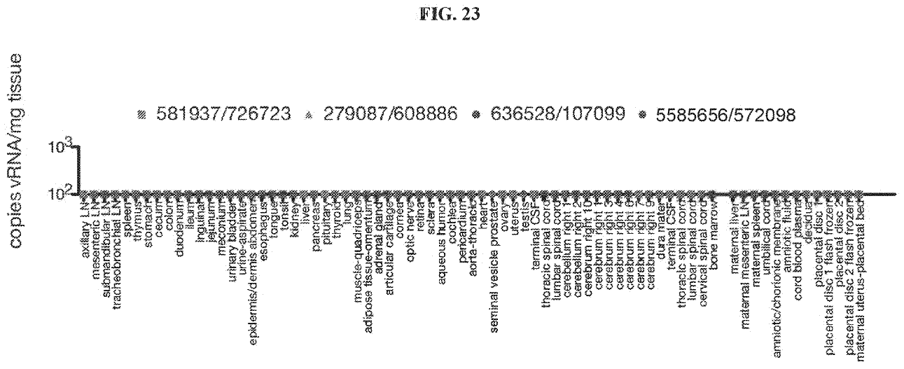

ZIKV-infected pregnant female rhesus macaque were treated with ZKV-IG (581937/729723 and 279087/608886) or placebo-IG (636528/107099 and 558656/572098). Following treatment, maternal, maternal/fetal, or fetal tissues were harvested and viral load was determined. There was no detectable virus in ZIKV-IG and placebo-IG treated maternal, maternal/fetal interface, or fetal tissues (FIG. 23). Similarly, no maternal, maternal/fetal interface or fetal tissues tested positive for viral RNA, mouse brain was used as positive control and water was used as negative control. A list of tissues/samples tested is shown in Tables 45-55.

TABLE 45
Viral Load Analysis in Tissues/Samples

| | Average | |
|---|---|---|
| | Viral | |
| | Load | |
| Subject Id | (“VL”) | Source Material |
| r18049 (or | 0 | liver |
| r18057) | | |
| r18049 (or | 0 | mesenteric LN |
| r18057) | | |
| r18049 (or | 0 | spleen |
| r18057) | | |
| 636528 | 0 | amniotic/chorionic membrane |
| 636528 | 0 | decidua - removed from placenta |
| 636528 | 0 | liver bx |
| 636528 | 0 | mesenteric LN |
| 636528 | 0 | placental disc 1 |
| 636528 | 0 | placental disc 1 flash frozen |
| 636528 | 0 | placental disc 2 |
| 636528 | 0 | placental disc 2 flash frozen |
| 636528 | 0 | spleen bx |
| 636528 | 0 | umbilical cord |
| 636528 | 0 | uterus-placental bed |
| 636528F | 0 | aorta-thoracic |
| 636528F | 0 | cervical spinal cord |
| 636528F | 0 | cochlea inside bony labyrinth - only |
| | | fixed |
| 636528F | 0 | cornea |
| 636528F | 0 | dura mater |
| 636528F | 0 | heart full thickness section |
| 636528F | 0 | lumbar spinal cord |
| 636528F | 0 | lung |
| 636528F | 0 | optic nerve |
| 636528F | 0 | pericardium |
| 636528F | 0 | retina |
| 636528F | 0 | sclera |
| 636528F | 0 | seminal vesicle-prostate/uterus |
| 636528F | 0 | thoracic spinal cord |
| water | 0. | negative control |
| mouse brain | 4.28E+10 | positive control |

* ZIKV-infected pregnant female rhesus macaque treated with non-specific human IG control (636528)
* 636528F-fetus of the corresponding mother

TABLE 46
Viral Load Analysis in Tissues

| | Average | |
|---|---|---|
| Subject Id | VL | Source Material |
| 581937F | 0. | axillary LN |
| 581937F | 0. | cerebellum 1 right |
| 581937F | 0. | cerebellum 2 right |
| 581937F | 0. | cerebrum 1 right |
| 581937F | 0. | cerebrum 10 right |
| 581937F | 0. | cerebrum 3 right |
| 581937F | 0. | cerebrum 4 right |
| 581937F | 0. | cerebrum 6 right |
| 581937F | 0. | cerebrum 7 right |
| 581937F | 0. | cerebrum 9 right |
| 581937F | 0. | inguinal LN |
| 581937F | 0. | kidney |
| 581937F | 0. | mesenteric LN |
| 581937F | 0. | muscle-quadriceps |
| 581937F | 0. | pituitary |
| 581937F | 0. | submandibular LN |
| 581937F | 0. | thymus |
| 581937F | 0. | thyroid |
| 581937F | 0. | tracheobroncial LN |
| 581937F | 0. | urinary bladder |
| 581937 | 0. | amniotic/chorionic membrane |
| 581937 | 0. | decidua |
| 581937 | 0. | placental disc 1 |
| 581937 | 0. | placental disc 1 - flash frozen |
| 581937 | 0. | placental disc 2 |
| 581937 | 0. | placental disc 2 - flash frozen |
| 581937 | 0. | umbilical cord |
| 581937 | 0. | uterus-placental bed |
| water | 0. | negative control |
| mouse brain | 2.36E+10 | positive control |

* ZIKV-infected pregnant female rhesus macaque treated with ZIKV-IG (581937)
* 581937F- fetus of the corresponding mother

TABLE 47
Viral Load Analysis in Tissues

| | Average | |
|---|---|---|
| Subject Id | VL | Source Material |
| 558656 | 0. | amniotic/chorionic membrane |
| 558656 | 0. | decidua |
| 558656 | 0. | placental disc 1 flash frozen |
| 558656 | 0. | placental disc 2 flash frozen |
| 558656 | 0. | umbilical cord |
| 558656 | 0. | uterus - placental bed |
| 558656F | 0. | adrenal gland |
| 558656F | 0. | bone marrow |
| 558656F | 0. | cecum |
| 558656F | 0. | cerebellum 1 right |
| 558656F | 0. | cerebellum 2 right |
| 558656F | 0. | cerebrum 1 right |
| 558656F | 0. | cerebrum 10 right |
| 558656F | 0. | cerebrum 3 right |
| 558656F | 0. | cerebrum 4 right |
| 558656F | 0. | cerebrum 6 right |
| 558656F | 0. | cerebrum 7 right |
| 558656F | 0. | cerebrum 9 right |
| 558656F | 0. | duodenum |
| 558656F | 0. | esophagus |
| 558656F | 0. | ileum |
| 558656F | 0. | meconium |
| 558656F | 0. | tongue |
| 558656F | 0. | tonsil |
| water | 0. | negative control |
| mouse brain | 6.75E+10 | positive control |

TABLE 48
Viral Load Analysis in Tissues

| | Average | |
|---|---|---|
| Subject Id | VL | Source Material |
| 636528F | 0. | adipose tissue - omentum |
| 636528F | 0. | articular cartilage |
| 636528F | 0. | bone marrow |
| 636528F | 0. | colon |
| 636528F | 0. | epidermis/dermis of abdomen |
| 636528F | 0. | jejunum |
| 636528F | 0. | liver |
| 636528F | 0. | pancreas |
| 636528F | 0. | spleen |
| 636528F | 0. | stomach |
| 636528F | 0. | submandibular LN |
| 636528F | 0. | testis |
| 636528F | 0. | thymus |
| 636528F | 0. | tracheobroncial LN |
| water | 0 | negative control |
| mouse brain | 3.85E+10 | positive control |

* ZIKV-infected pregnant female rhesus macaque treated with non-specific human IG control (636528)
* 636528F-fetus of the corresponding mother

TABLE 49
Viral Load Analysis in Tissues

| | Average | |
|---|---|---|
| Subject Id | VL | Source Material |
| 608886 (279087F) | 0. | aorta-thoracic |
| 608886 (279087F) | 0. | articular cartilage |
| 608886 (279087F) | 0. | bone marrow |
| 608886 (279087F) | 0. | cervical spinal cord |
| 608886 (279087F) | 0. | cochlea |
| 608886 (279087F) | 0. | cornea |
| 608886 (279087F) | 0. | dura mater |
| 608886 (279087F) | 0. | heart |
| 608886 (279087F) | 0. | lumbar spinal cord |
| 608886 (279087F) | 0. | lung |
| 608886 (279087F) | 0. | optic nerve |
| 608886 (279087F) | 0. | ovary |
| 608886 (279087F) | 0. | pericardium |
| 608886 (279087F) | 0. | retina |
| 608886 (279087F) | 0. | sclera |
| 608886 (279087F) | 0. | thoracic spinal cord |
| 608886 (279087F) | 0. | uterus |
| 726723 (581937F) | 0. | adrenal gland |
| 726723 (581937F) | 0. | cecum |
| 726723 (581937F) | 0. | duodenum |
| 726723 (581937F) | 0. | esophagus |
| 726723 (581937F) | 0. | ileum |
| 726723 (581937F) | 0. | meconium |
| 726723 (581937F) | 0. | tongue |
| 726723 (581937F) | 0. | tonsil |
| 279087 | 0. | liver |
| 279087 | 0. | mesenteric LN |
| 279087 | 0. | spleen bx |
| water | 0. | negative control |
| mouse brain | 3.59E+10 | positive control |

* ZIKV-infected pregnant female rhesus macaque treated with ZIKV-IG (581937 and 279087)
* 581937F and 279087F- fetus of the corresponding mother

TABLE 50
Viral Load Analysis in Tissues

| | Average | |
|---|---|---|
| Subject Id | VL | Source Material |
| r09055 | 0. | liver |
| r09055 | 0. | mesenteric LN |
| r09055 | 0. | spleen |
| 636528F | 0. | adrenal gland |
| 636528F | 0. | cecum |
| 636528F | 0. | cerebellum 1 right |
| 636528F | 0. | cerebellum 2 right |
| 636528F | 0. | cerebrum 1 right |
| 636528F | 0. | cerebrum 10 right |
| 636528F | 0. | cerebrum 3 right |
| 636528F | 0. | cerebrum 4 right |
| 636528F | 0. | cerebrum 6 right |
| 636528F | 0. | cerebrum 7 right |
| 636528F | 0. | cerebrum 9 right |
| 636528F | 0. | duodenum |
| 636528F | 0. | esophagus |
| 636528F | 0. | ileum |
| 636528F | 0. | kidney |
| 636528F | 0. | meconium |
| 636528F | 0. | pituitary |
| 636528F | 0. | thyroid |
| 636528F | 0. | tongue |
| 636528F | 0. | urinary bladder |
| 636528F | 0. | tonsil |

* ZIKV-infected pregnant female rhesus macaque treated with non-specific human IG control (636528)
* 636528F-fetus of the corresponding mother

TABLE 51
Viral Load Analysis in Tissues

| | | |
|---|---|---|
| | Average | |
| Subject Id | VL | Source Material |

tissues - in portal/slack

| | | |
|---|---|---|
| 608886 (279087F) | 0. | adipose tissue omentum |
| 608886 (279087F) | 0. | axillary LN |
| 608886 (279087F) | 0. | cerebrum 1 right |
| 608886 (279087F) | 0. | colon |
| 608886 (279087F) | 0. | epidermis/dermis abdomen |
| 608886 (279087F) | 0. | inguinal LN |
| 608886 (279087F) | 0. | jejunum |
| 608886 (279087F) | 0. | kidney |
| 608886 (279087F) | 0. | liver |
| 608886 (279087F) | 0. | mesenteric LN |
| 608886 (279087F) | 0. | muscle-quadriceps |
| 608886 (279087F) | 0. | pancreas |
| 608886 (279087F) | 0. | pituitary |
| 608886 (279087F) | 0. | spleen |
| 608886 (279087F) | 0. | stomach |
| 608886 (279087F) | 0. | submandibular LN |
| 608886 (279087F) | 0. | thymus |
| 608886 (279087F) | 0. | thyroid |
| 608886 (279087F) | 0. | tracheobronchial LN |
| 608886 (279087F) | 0. | urinary bladder |
| 279087 | 0. | amniotic/chorionic membrane |
| 279087 | 0. | decidua |
| 279087 | 0. | placental disc 1 |
| 279087 | 0. | placental disc 1 F |
| 279087 | 0. | placental disc 2 |
| 279087 | 0. | placental disc 2 F |
| 279087 | 0. | umbilical cord |
| 279087 | 0. | uterus-placental bed |
| water | 0. | negative control |
| mouse brain | 4.72E+10 | positive control |

* ZIKV-infected pregnant female rhesus macaque treated with ZIKV-IG (279087)
* 279087F- fetus of the corresponding mother

TABLE 52
Viral Load Analysis in Tissues

| | Average | |
|---|---|---|
| Subject Id | VL | Source Material |
| 636528F | 0. | axillary LN |
| 636528F | 0. | inguinal LN |
| 636528F | 0. | mesenteric LN |
| 636528F | 0. | muscle-quadriceps |
| 636528F | 0. | tonsil |
| mouse brain | 1.86E+10 | 16.0600 |
| Water** | 1.58E+02 | 39.5000 |

* ZIKV-infected pregnant female rhesus macaque treated with non-specific human IG control (636528)
* 636528F-fetus of the corresponding mother
**some contamination in water from tissue extraction noted

TABLE 53
Viral Load Analysis in Tissues

| | Average | |
|---|---|---|
| Subject Id | VL | Source Material |
| 279087F | 0. | adrenal gland |
| 279087F | 0. | cecum |
| 279087F | 0. | cerebellum 1 right |
| 279087F | 0. | cerebellum 2 right |
| 279087F | 0. | cerebrum 10 right |
| 279087F | 0. | cerebrum 3 right |
| 279087F | 0. | cerebrum 4 right |
| 279087F | 0. | cerebrum 6 right |
| 279087F | 0. | cerebrum 7 right |
| 279087F | 0. | cerebrum 9 right |
| 279087F | 0. | duodenum |
| 279087F | 0. | esophagus |
| 279087F | 0. | ileum |
| 279087F | 0. | meconium |
| 279087F | 0. | tongue |
| 279087F | 0. | tonsil |
| 558656 | 0. | liver |
| 558656 | 0. | mesenteric LN |
| 558656 | 0. | spleen bx |
| 558656F | 0. | cervical spinal cord |
| 558656F | 0. | cochlea |
| 558656F | 0. | cornea |
| 558656F | 0. | dura mater |
| 558656F | 0. | lumbar spinal cord |
| 558656F | 0. | optic nerve |
| 558656F | 0. | retina |
| 558656F | 0. | sclera |
| 558656F | 0. | thoracic spinal cord |
| water | 42.12 | negative control |
| mouse brain | 4.84E+10 | positive control |

* ZIKV-infected pregnant female rhesus macaque treated with ZIKV-IG (279087)
* 279087F- fetus of the corresponding mother

TABLE 54
Viral Load Analysis in Tissues

| | Average | |
|---|---|---|
| Subject Id | VL | Source Material |
| 581937 | 0. | liver |
| 581937 | 0. | mesenteric LN |
| 581937 | 0. | spleen bx |
| 581937F | 0. | adipose tissue-omentum |
| 581937F | 0. | aorta-thoracic |
| 581937F | 0. | articular cartilage |
| 581937F | 0. | bone marrow |
| 581937F | 0. | cervical spinal cord |
| 581937F | 0. | cochlea |
| 581937F | 0. | colon |
| 581937F | 0. | cornea |
| 581937F | 0. | dura mater |
| 581937F | 0. | epidermis/dermis abdomen |
| 581937F | 0. | heart |
| 581937F | 0. | jejunum |
| 581937F | 0. | liver |
| 581937F | 0. | lumbar spinal cord |
| 581937F | 0. | lung |
| 581937F | 0. | optic nerve |
| 581937F | 0. | ovary |
| 581937F | 0. | pancreas |
| 581937F | 0. | pericardium |
| 581937F | 0. | retina |
| 581937F | 0. | sclera |
| 581937F | 0. | spleen |
| 581937F | 0. | stomach |
| 581937F | 0. | thoracic spinal cord |
| 581937F | 0. | uterus |
| water | 0. | negative control |
| mouse brain | 7.83E+10 | positive control |

* ZIKV-infected pregnant female rhesus macaque treated with ZIKV-IG (581937)
* 581937F- fetus of the corresoondins mother

TABLE 55
Viral Load Analysis in Tissues

| | Average | |
|---|---|---|
| Subject Id | VL | Source Material |
| 558656 | 0. | placental disc 1 |
| 558656 | 0. | placental disc 2 |
| 558656F | 0. | adipose tissue - omentum |
| 558656F | 0. | aorta-thoracic |
| 558656F | 0. | articular cartilage |
| 558656F | 0. | axillary LN |
| 558656F | 0. | colon |
| 558656F | 0. | epidermis/dermis abdomen |
| 558656F | 0. | heart |
| 558656F | 0. | inguinal LN |
| 558656F | 0. | jejunum |
| 558656F | 0. | kidney |
| 558656F | 0. | liver |
| 558656F | 0. | lung |
| 558656F | 0. | mesenteric LN |
| 558656F | 0. | muscle-quadriceps |
| 558656F | 0. | ovary |
| 558656F | 0. | pancreas |
| 558656F | 0. | pericardium |
| 558656F | 0. | pituitary gland |
| 558656F | 0. | spleen |
| 558656F | 0. | stomach |
| 558656F | 0. | submandibular LN |
| 558656F | 0. | thymus |
| 558656F | 0. | thyroid |
| 558656F | 0. | tracheobronchial LN |
| 558656F | 0. | urinary bladder |
| 558656F | 0. | uterus |
| water | 0. | Negative control |
| mouse brain | 6.73E+10 | positive control CTL |

### Serum ZKV-IG PK Analysis

Binding antibody titers were quantitated using whole virion ZIKV ELISA. Briefly, high-binding ELISA plates were coated with 30 ng of 4G2 antibody (clone D1-4G2-4-15) overnight at 40° C. After plate blocking, whole Zika virus virions were captured by the antibodies for 1 hour. Serum from macaques or stock ZIKV-IG as a standard were serially diluted and incubated on the plates. Serum IgG were tagged with horseradish peroxidase-conjugated anti-monkey IgG antibody and/or anti-human IgG antibody and optical densities were detected at 450 nm. The log₁₀ 50% effective dilution (ED₅₀) was calculated for IgG binding responses against the whole virion. Optical densities were converted to concentration using the known concentration of the ZIKV-IG standard. Table 56 shows the ELISA results of serum human IgG.

TABLE 56
ELISA results of serum human IgG

| | | | | | Concen- |
|---|---|---|---|---|---|
| | Days Post | Days Post | Hours Post | Hours Post | tration |
| Animal | Infection | Infusion | Infection | Infusion | (ug/mL) |
| 581937 | 0 | −1 | 0 | −24 | 47.60 |
| 581937 | 1 | 0 | 24 | 0 | 47.60 |
| 581937 | 1.042 | 0.042 | 25 | 1 | 1560.24 |
| 581937 | 1.25 | 0.25 | 30 | 6 | 787.05 |
| 581937 | 2 | 1 | 48 | 24 | 680.03 |
| 581937 | 3 | 2 | 72 | 48 | 633.98 |
| 581937 | 4 | 3 | 96 | 72 | 425.57 |
| 581937 | 5 | 4 | 120 | 96 | 371.56 |
| 581937 | 5.042 | 4.042 | 121 | 97 | 1114.91 |
| 581937 | 5.25 | 4.25 | 126 | 102 | 1598.88 |
| 581937 | 6 | 5 | 144 | 120 | 1287.68 |
| 581937 | 7 | 6 | 168 | 144 | 1822.66 |
| 581937 | 9 | 8 | 216 | 192 | 373.60 |
| 581937 | 13 | 12 | 312 | 288 | 827.06 |
| 581937 | 16 | 15 | 384 | 360 | 642.50 |
| 581937 | 20 | 19 | 480 | 456 | 303.30 |
| 581937 | 23 | 22 | 552 | 528 | 260.01 |
| 581937 | 27 | 26 | 648 | 624 | 222.26 |
| 581937 | 30 | 29 | 720 | 696 | 244.75 |
| 581937 | 34 | 33 | 816 | 792 | 169.72 |
| 581937 | 37 | 36 | 888 | 864 | 143.06 |
| 581937 | 41 | 40 | 984 | 960 | 52.39 |
| 581937 | 44 | 43 | 1056 | 1032 | 47.60 |
| 581937 | 48 | 47 | 1152 | 1128 | 47.60 |
| 581937 | 55 | 54 | 1320 | 1296 | 47.60 |
| 581937 | 62 | 61 | 1488 | 1464 | 47.60 |
| 279087 | 0 | −1 | 0 | −24 | 47.60 |
| 279087 | 1 | 0 | 24 | 0 | 47.60 |
| 279087 | 1.042 | 0.042 | 25 | 1 | 639.51 |
| 279087 | 1.25 | 0.25 | 30 | 6 | 1034.03 |
| 279087 | 2 | 1 | 48 | 24 | 812.91 |
| 279087 | 3 | 2 | 72 | 48 | 702.29 |
| 279087 | 4 | 3 | 96 | 72 | 429.97 |
| 279087 | 5 | 4 | 120 | 96 | 384.32 |
| 279087 | 5.042 | 4.042 | 121 | 97 | 581.54 |
| 279087 | 5.25 | 4.25 | 126 | 102 | 1101.31 |
| 279087 | 6 | 5 | 144 | 120 | 1112.86 |
| 279087 | 7 | 6 | 168 | 144 | 1166.86 |
| 279087 | 9 | 8 | 216 | 192 | 1273.33 |
| 279087 | 13 | 12 | 312 | 288 | 711.82 |
| 279087 | 16 | 15 | 384 | 360 | 604.91 |
| 279087 | 20 | 19 | 480 | 456 | 90.70 |
| 279087 | 23 | 22 | 552 | 528 | 80.61 |
| 279087 | 27 | 26 | 648 | 624 | 51.31 |
| 279087 | 30 | 29 | 720 | 696 | 47.60 |
| 279087 | 34 | 33 | 816 | 792 | 47.60 |
| 279087 | 37 | 36 | 888 | 864 | 47.60 |
| 279087 | 41 | 40 | 984 | 960 | 47.60 |
| 279087 | 44 | 43 | 1056 | 1032 | 47.60 |
| 279087 | 48 | 47 | 1152 | 1128 | 47.60 |
| 279087 | 55 | 54 | 1320 | 1296 | 47.60 |

### Serum Anti-ZIKV Antibody Titer Analysis

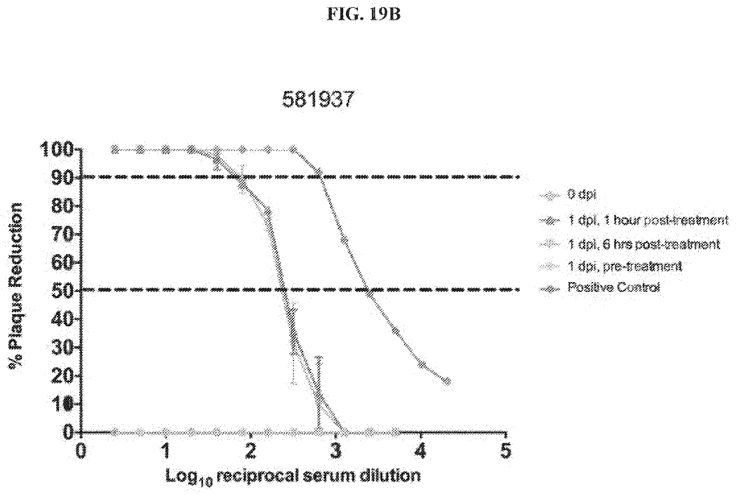

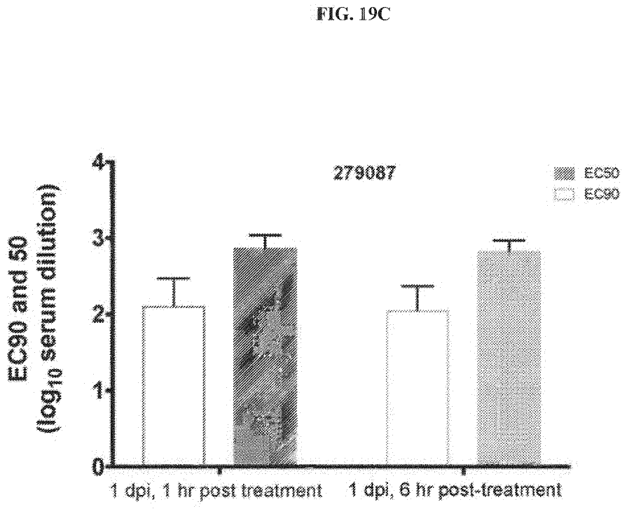

PRNT assays were performed using serum collected at various time points. These time points included 1 and 6 hours after the first human immunoglobulin (HIG) injection, days 2-5, day 5 at 1 and 6 hours after the second HIG injection and days 6 and 7. For the assays, serum was serially diluted, combined with 200 PFU of PRVABC59, and plated with Vero cells. After incubation of the virus and serum with the cells, the cultures were overlaid with agar and stained with Neutral red at day 4 or 5. Plaques were counted daily until they no longer increased infrequency. PRNT₅₀ (EC₅₀) and PRNT₉₀(EC₉₀) values were assessed. PRNT₉ is the highest dilution of serum that results in a 90% reduction of plaques compared to aback-titrated virus control. And PRNT₅₀ is the highest dilution of serum that results in a 50% reduction of plaques compared to aback-titrated virus control. Results are shown in FIG. 19A, FIG. 19B, and FIG. 19C and the PRNT₅ and PRNT₉₀ values obtained for data up to Day 7 are shown in Table 57.

TABLE 57
PRNT₅₀ and PRNT₉₀ values for ZIKV-IG treated animals

| | Days Post | Days Post | Hours Post | Hours Post | PRNT90 | PRNT50 |
|---|---|---|---|---|---|---|
| Animal | Infection | Infusion | Infection | Infusion | (log10) | (log10) |
| 581937 | 1.042 | 0.042 | 25 | 1 | 1.98 | 2.406 |
| 581937 | 1.25 | 0.25 | 30 | 6 | 1.96 | 2.368 |
| 581937 | 2 | 1 | 48 | 24 | 1.83 | 2.519 |
| 581937 | 3 | 2 | 72 | 48 | 1.82 | 2.563 |
| 581937 | 4 | 3 | 96 | 72 | 1.83 | 2.596 |
| 581937 | 5 | 4 | 120 | 96 | 1.74 | 2.36 |
| 581937 | 5.042 | 4.042 | 121 | 97 | 2.08 | 2.787 |
| 581937 | 5.25 | 4.25 | 126 | 102 | 2.23 | 2.884 |
| 581937 | 6 | 5 | 144 | 120 | 1.81 | 2.549 |
| 581937 | 7 | 6 | 168 | 144 | 1.81 | 2.671 |
| 581937 | 9 | 8 | 216 | 192 | 1.87 | 2.58 |
| 581937 | 13 | 12 | 312 | 288 | 1.79 | 2.6 |
| 581937 | 16 | 15 | 384 | 360 | 1.64 | 2.549 |
| 581937 | 20 | 19 | 480 | 456 | 1.41 | 2.671 |
| 581937 | 27 | 26 | 648 | 624 | 1.48 | 2.024 |
| 581937 | 34 | 33 | 816 | 792 | 0.93 | 1.19 |
| 581937 | 41 | 40 | 984 | 960 | 1.17 | 1.353 |
| 581937 | 55 | 54 | 1320 | 1296 | 1.65 | 1.94 |
| 581937 | 62 | 61 | 1488 | 1464 | 1.80 | 2.085 |
| 279087 | 1.042 | 0.042 | 25 | 1 | 2.11 | 2.874 |
| 279087 | 1.25 | 0.25 | 30 | 6 | 2.05 | 2.826 |
| 279087 | 2 | 1 | 48 | 24 | 1.43 | 1.923 |
| 279087 | 3 | 2 | 72 | 48 | 1.17 | 1.558 |
| 279087 | 4 | 3 | 96 | 72 | 1.24 | 1.689 |
| 279087 | 5 | 4 | 120 | 96 | 1.43 | 2.531 |
| 279087 | 5.042 | 4.042 | 121 | 97 | 1.60 | 2.55 |
| 279087 | 5.25 | 4.25 | 126 | 102 | 1.84 | 2.69 |
| 279087 | 6 | 5 | 144 | 120 | 1.64 | 2.025 |
| 279087 | 7 | 6 | 168 | 144 | 1.27 | 1.625 |
| 279087 | 9 | 8 | 216 | 192 | 1.34 | 1.95 |
| 279087 | 13 | 12 | 312 | 288 | 1.24 | 2.031 |
| 279087 | 16 | 15 | 384 | 360 | 1.04 | 1.312 |
| 279087 | 20 | 19 | 480 | 456 | 0.86 | 1.183 |
| 279087 | 27 | 26 | 648 | 624 | 1.18 | 1.889 |
| 279087 | 34 | 33 | 816 | 792 | 0.00 | 0.783 |
| 279087 | 41 | 40 | 984 | 960 | 0.49 | 0.8482 |
| 279087 | 55 | 54 | 1320 | 1296 | 2.32 | 2.688 |
| 279087 | 62 | 61 | 1488 | 1464 | 2.51 | 3.465 |
| 518832 | 1.042 | 0.042 | 25 | 1 | 1.6 | 2.019 |
| 518832 | 1.25 | 0.25 | 30 | 6 | 1.5 | 1.973 |
| 518832 | 2 | 1 | 48 | 24 | 1.3 | 1.97 |
| 518832 | 3 | 2 | 72 | 48 | 1.3 | 2.005 |
| 518832 | 4 | 3 | 96 | 72 | 1.2 | 1.881 |
| 518832 | 5 | 4 | 120 | 96 | 1.2 | 1.876 |
| 518832 | 5.042 | 4.042 | 121 | 97 | 1.9 | 2.63 |
| 518832 | 5.25 | 4.25 | 126 | 102 | 1.6 | 2.096 |
| 518832 | 6 | 5 | 144 | 120 | 1.6 | 2.028 |
| 518832 | 7 | 6 | 168 | 144 | 1.6 | 2.099 |
| 518832 | 9 | 8 | 216 | 192 | 1.5 | 2.234 |
| 518832 | 13 | 12 | 312 | 288 | 1.4 | 2.001 |
| 518832 | 16 | 15 | 384 | 360 | 1.3 | 1.818 |

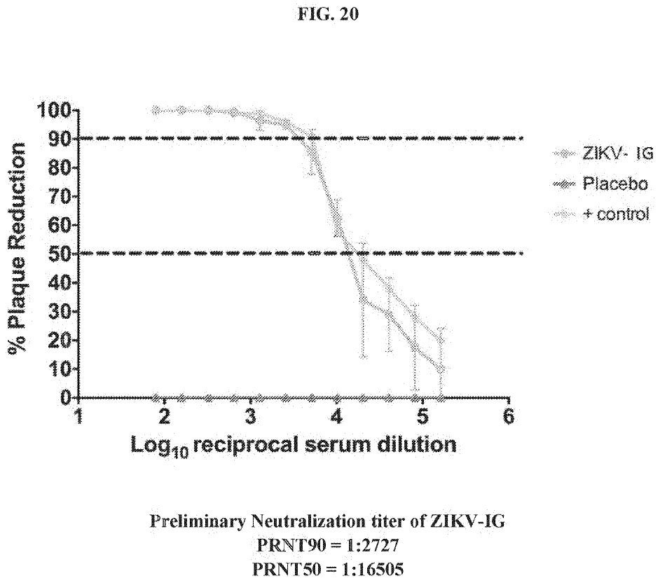

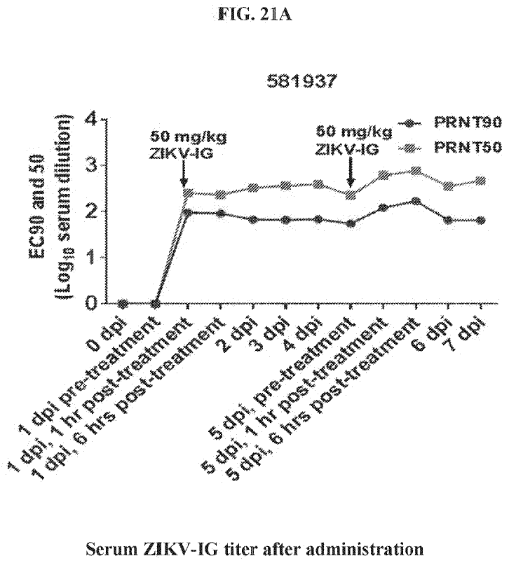

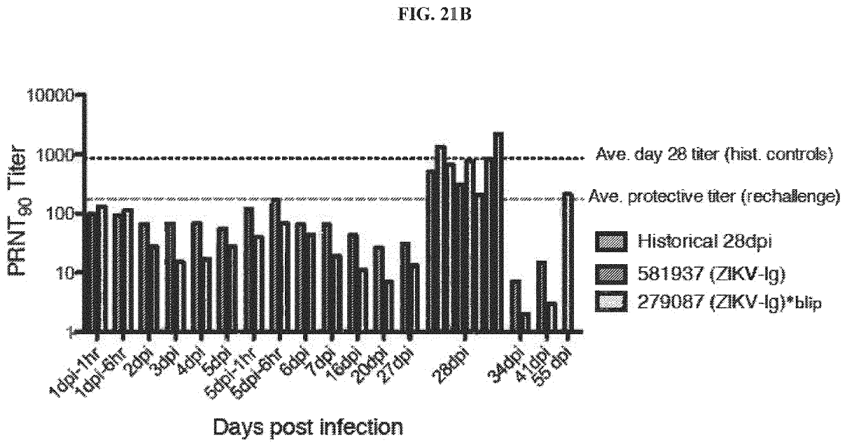

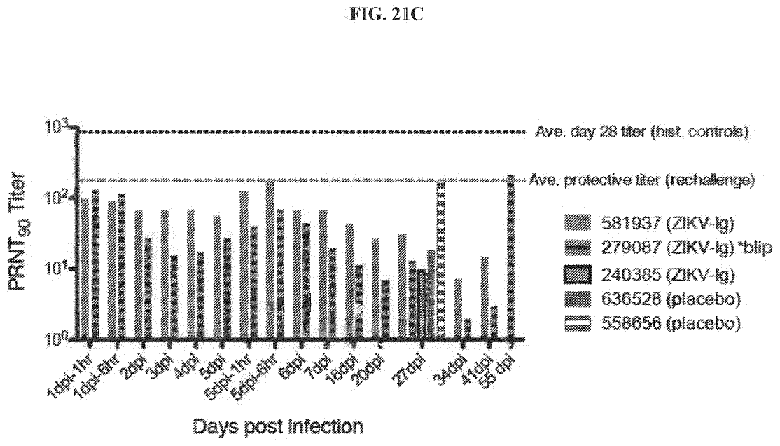

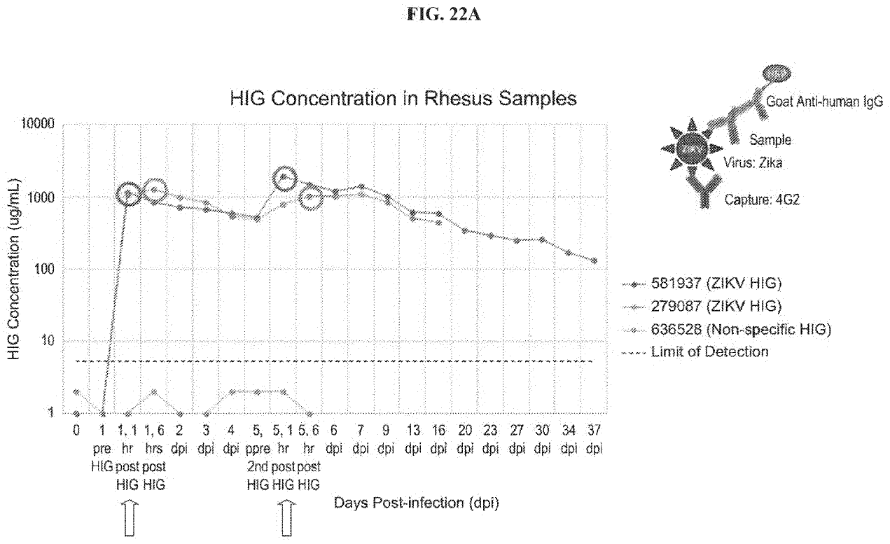

In addition, FIG. 20 shows the preliminary neutralization titer values (PRNT₉₀ and PRNT₅₀) prior to administration of 50 mg/kg ZIKV-IG, placebo or positive control. The preliminary reduction neutralization test (PRNT) values were PRNT₉₀=1:2727 and PRNT₅₀=1:16505. FIG. 21A shows serum ZIKV-IG titer values (PRNT90 and PRNT50) over time taken at 0 day post-infection (dpi), 1 dpi pre-treatment with ZIKV-IG, 1 dpi at 1 hour and 6 hours after a first 50 mg/kg ZIKV-IG administration, 2-4 dpi, 5 dpi prior to the second ZIKV-IG administration, 5 dpi at 1 hour and 6 hours after a second 50 mg/kg ZIKV-IG administration, and 6-7 dpi from a ZIKV-infected pregnant female rhesus macaque (581937). FIG. 21B shows serum ZIKV-IG titer values (PRNT90) over time taken at 1 and 6 hours after the first ZIKV-IG administration, days 2-5, day 5 at 1 and 6 hours after the second ZIKV-IG administration, and days 6, 7, 16, 20, 27, 34, 41, and 55 from ZIKV-infected pregnant female rhesus macaques (279087 and 581937); and historical controls at day 28 post-infection. ZIKV-infected pregnant macaque 581937 reached the average titer of animals protected from re-challenge (FIG. 21i). FIG. 21C shows serum ZIKV-IG titer values (PRNT90) over time taken at 1 and 6 hours after the first ZIKV-IG administration, days 2-5, day 5 at 1 and 6 hours after the second ZIKV-IG administration, and days 6, 7, 16, 20, 27, 34, 41, and 55 from ZIKV-infected pregnant female rhesus macaques (581937 and 279087); and ZIKV-infected pregnant female rhesus macaque (240385) and placebo (636528 and 558656) at day 27 post-infection. ZIKV-infected pregnant macaque 581937 reached the average titer of animals protected from re-challenge (FIG. 21C).

### Estimated Half-life

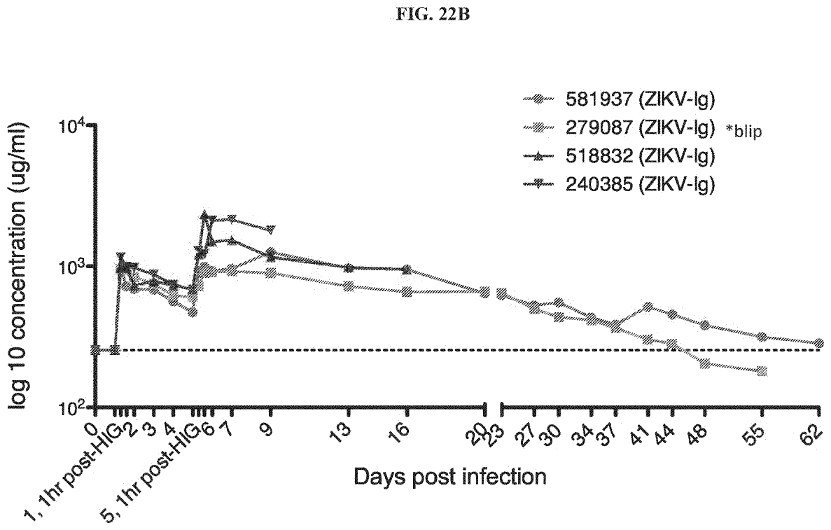

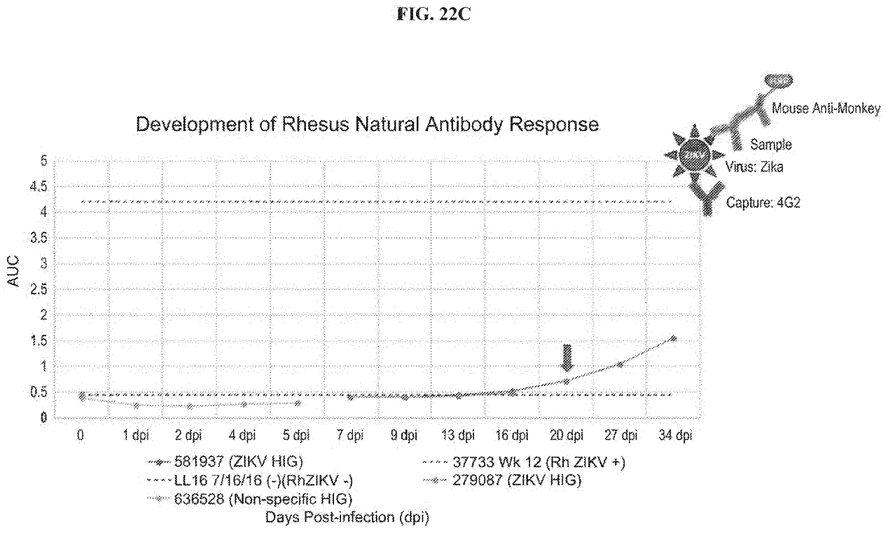

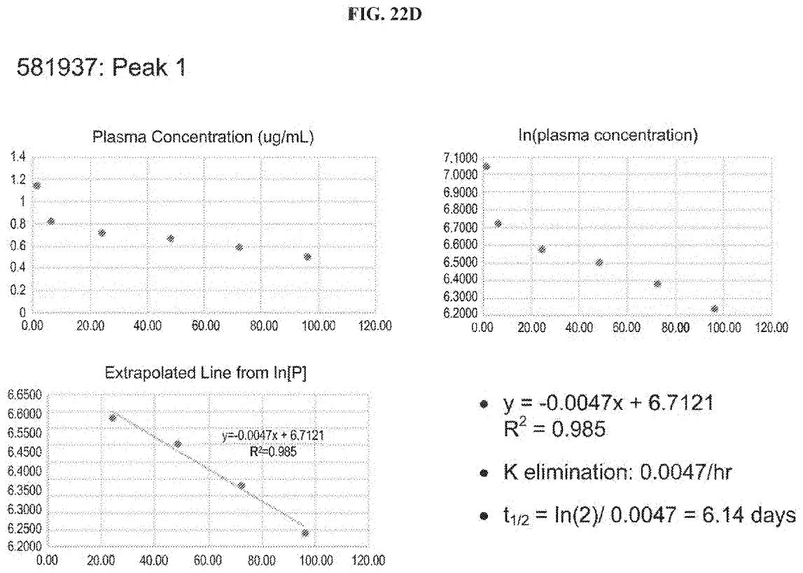

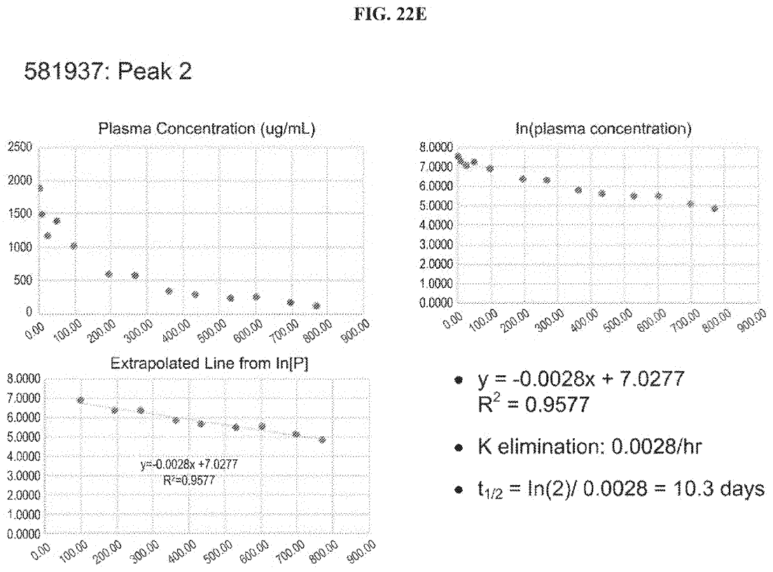

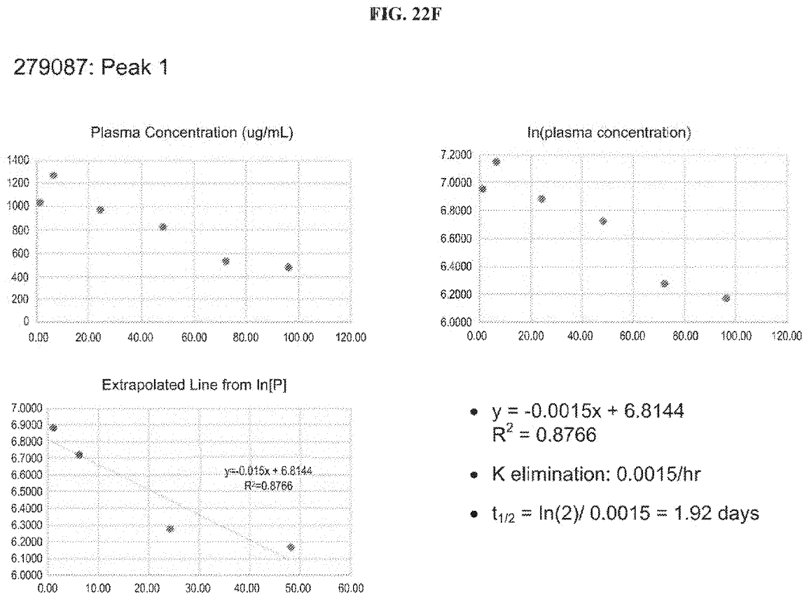

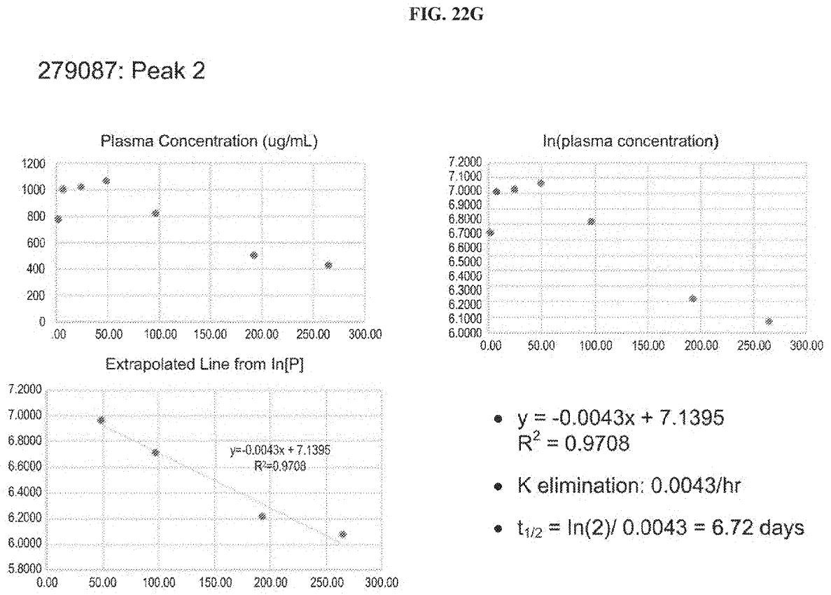

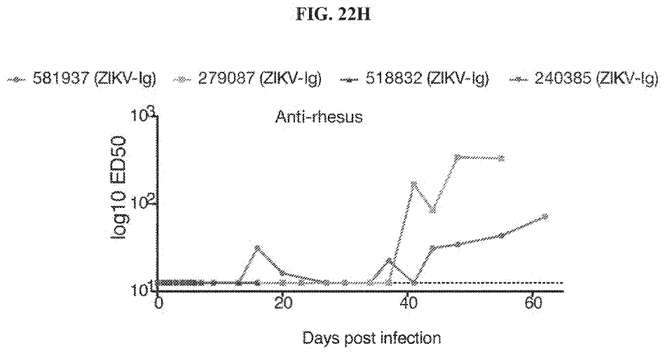

Antibody concentrations measured by whole virion binding ELISA showed the estimated half-life of the human-derived ZIKV-IG ˜10 days and detected a macaque-specific de novo antibody response starting at ˜7-13 dpi in non-specific IG treated animals (placebo) and ˜20 dpi in ZIKV-IG treated animal. Summary of the preliminary results for ZIKV-IG concentration over time and half-life results are shown in Table 58 and FIG. 22A, FIG. 22B, FIG. 22C, FIG. 22D, FIG. 22E, FIG. 22F, and FIG. 22G.

TABLE 58
Summary of human IG estimated half-life (Non-compartmental

| | | | | | | | | | | | Half_life |
|---|---|---|---|---|---|---|---|---|---|---|---|
| Animal | AUCL | Cmax | Tmax | Clast | Tlast | Lambda | Zika_Dose | AUCinf | Clearance | Vz | (hr) |
| 279087 | 392.48785 | 1.2733292 | 192.00 | 0.0475992 | 1464 | 0.00235 | 356.4 | 166708.88 | 0.0021379 | 0.9079435 | 294.37796 |
| r03125 | 200.21271 | 3.6149197 | 97.00 | 0.0475992 | 1464 | 0.00239 | 451.44 | 83750.558 | 0.0053903 | 2.2542659 | 289.88002 |
| 240385 | 41.120823 | 0.376279 | 102.00 | 0.0475992 | 1464 | 0.00126 | 307.26 | 32671.042 | 0.0094047 | 7.4634874 | 550.07794 |
| 581937 | 379.93203 | 1.822662 | 144.00 | 0.0475992 | 1464 | 0.00269 | 410.4 | 141307.16 | 0.0029043 | 1.0800579 | 257.76818 |

FIG. 22A, FIG. 22B, FIG. 22C, FIG. 22D, FIG. 22E, FIG. 22F, and FIG. 22G show preliminary results for antibody concentrations measured by ELISA and estimated half-life of human ZIKV-IG. FIG. 22A shows human IG (HIG) concentration in samples from ZIKV-infected pregnant female rhesus macaque treated with ZIKV-IG (581937 and 279087) or non-specific human IG control (636528). FIG. 22B shows preliminary human IG concentration in serum samples from ZIKV-infected pregnant female rhesus macaque treated with ZIKV-IG (581937, 279087, 518832, and 240385) over time post-infection. FIG. 22C shows natural antibody response in ZIKV-infected pregnant female rhesus macaque treated with ZIKV-IG (581937 and 279087) or non-specific human IG control (r10093). FIG. 22D-22G show estimated half-life calculations for peak 1 and peak 2 of human ZIKV-IG in ZIKV-infected pregnant female rhesus macaque treated with ZIKV-IG (581937 and 279087). FIG. 22H shows rhesus IG concentration in serum samples from ZIKV-infected pregnant female rhesus macaque treated with ZIKV-IG (581937, 279087, 518832, and 240385) over time post-infection.

These studies suggest that ZIKV-IG can reduce maternal plasma viremia upon administration relative to untreated controls, and may be useful as post-exposure prophylaxis in pregnancy.

What is claimed is:

1. A method for treating, preventing, or reducing the risk of a Zika virus infection, the method comprising: administering to a subject in need thereof an effective amount of a composition comprising Zika virus neutralizing polyclonal antibodies, wherein the polyclonal antibodies are from pooled plasma and/or serum from mammalian donors.

2. A method for reducing viral load of Zika virus in a bodily fluid, tissue, or cell of a subject, the method comprising: administering to the subject an effective amount of a composition comprising Zika virus neutralizing polyclonal antibodies, wherein the polyclonal antibodies are from pooled plasma and/or serum from mammalian donors.

3. A method for increasing antibody titers to Zika virus in a bodily fluid, tissue, or cell of a subject, the method comprising: administering to the subject an effective amount of a composition comprising Zika virus neutralizing polyclonal antibodies, wherein the polyclonal antibodies are from pooled plasma and/or serum from mammalian donors.

4. A method of eliciting an immune response against Zika virus comprising: administering an effective amount of a composition comprising Zika virus polyclonal antibodies to a subject, wherein the polyclonal antibodies are from pooled plasma and/or serum from mammalian donors.

5. A method of passive immunization against a Zika virus, the method comprising: administering an effective amount of a composition comprising Zika virus polyclonal antibodies to a subject, wherein the polyclonal antibodies are from pooled plasma and/or serum from mammalian donors.

6. The method of any one of the previous claims, wherein the subject is male.

7. The method of any one of claims 1-5, wherein the subject is pregnant, suspected of being pregnant, or trying to become pregnant with a fetus.

8. A method of preventing or reducing the risk of transmission of a Zika virus infection from a subject to an embryo, fetus, or infant, the method comprising: administering to the subject an effective amount of a composition comprising Zika virus neutralizing polyclonal antibodies, wherein the polyclonal antibodies are from pooled plasma and/or serum from mammalian donors.

9. A method of preventing or reducing the risk of transmission of a Zika virus from a subject, the method comprising: administering to the subject an effective amount of a composition comprising Zika virus neutralizing polyclonal antibodies, wherein the polyclonal antibodies are from pooled plasma and/or serum from mammalian donors, and wherein the subject is trying to become pregnant or is of age to become pregnant.

10. A method of treating, preventing, or reducing the risk of a Zika virus infection in an embryo or a fetus, the method comprising: administering an effective amount of a composition comprising Zika virus neutralizing polyclonal antibodies to a subject pregnant with the embryo or the fetus, wherein the polyclonal antibodies are from pooled plasma and/or serum from mammalian donors.

11. A method of preventing or reducing the severity or risk of microcephaly in a fetus comprising: administering to a pregnant subject carrying the fetus an effective amount of a composition comprising Zika virus neutralizing polyclonal antibodies, wherein the polyclonal antibodies are from pooled plasma and/or serum from mammalian donors.

12. The method of any one of the previous claims, wherein the mammalian donors are human.

13. The method of any one of the previous claims, wherein the polyclonal antibodies are from pooled plasma of one or more human donors.

14. The method of any one of the previous claims, wherein the mammalian donors were infected with Zika virus prior to pooling plasma and/or serum.

15. The method of any one of the previous claims, wherein the mammalian donors were vaccinated with Zika vaccine prior to pooling plasma and/or serum.

16. The method of any one of the previous claims, wherein the mammalian donors have elevated levels of anti-Zika virus antibodies.

17. The method of any one of the previous claims, wherein the mammalian donors have elevated levels of antibodies against a Zika Non-Structural protein 1 (anti-NS1 antibody) and/or a Zika Envelope protein (anti-E-protein antibody).

18. The method of any one of the previous claims, wherein the polyclonal antibodies comprise IgG antibodies.

19. The method of claim 18, wherein the IgG antibodies are greater than 95% of the antibody content of the composition.

20. The method of any one of the previous claims, wherein the effective amount is sufficient to provide a Zika virus antigen-specific immune response in the subject.

21. The method of any one of the previous claims, wherein the effective amount is sufficient to neutralize the Zika virus in the subject.

22. The method of any one of the previous claims, wherein the subject is pregnant and transmission of Zika virus from the pregnant subject to the embryo or the fetus is prevented, reduced or eliminated.

23. The method of any one of the previous claims, wherein the subject is pregnant and the effective amount is sufficient to provide a Zika virus antigen-specific immune response in the fetus.

24. The method of any one of claims 7-23 which comprises passive immunization of the fetus.

25. The method of any one of claims 7-24, wherein the subject and fetus are human.

26. The method of any one of claims 7-25, wherein the subject is in the first trimester, second trimester or third trimester of pregnancy.

27. The method of any one of claims 7-26, wherein the subject is in the late stage of the first trimester or early stage of the second trimester of pregnancy.

28. The method of any one of claims 7-27, wherein the risk of miscarriage and/or stillbirth is reduced.

29. The method of any one of the previous claims, wherein the subject has been bitten by a mosquito suspected of harboring the Zika virus, lives in an area that has a Zika virus outbreak, is visiting or has visited an area that has a Zika virus outbreak, is immunocompromised, is suspected of having been exposed to a person harboring the Zika virus, has come into physical contact or close physical proximity with an infected individual, is a hospital employee, and/or lives in or is visiting a country or region known to have mosquitoes harboring the Zika virus.

30. The method of any one of the previous claims, wherein the composition is administered to the subject before the subject has been infected with the Zika virus, after the subject has been infected with the Zika virus, or after the subject has been exposed to or is suspected of having been exposed to the Zika virus and before the Zika virus infection can be detected.

31. The method of any one of the previous claims, wherein the subject has been diagnosed with having or is suspected of having African lineage Zika virus strain, Asian lineage Zika virus strain, Brazil lineage virus strain, or Puerto Rico lineage virus strain.

32. The method of any one of the previous claims, wherein the subject has been diagnosed with having or is suspected of having Zika virus strain MR 766, FLR, Brazil-ZKV2015, or PRVABC59.

33. The method of any one of the previous claims, wherein the administration treats, prevents or reduces the risk of one or more symptoms associated with Zika virus infection.

34. The method of claim 33, wherein the one or more symptoms associated with the Zika virus infection comprise a fever, rash, headache, joint pain, conjunctivitis, or muscle pain.

35. The method of any one of the previous claims, wherein the administration is intravenous, intramuscular, subcutaneous, or intrauterinal.

36. The method of any one of the previous claims, wherein the composition is administered as at least one dose of about 50 mg/kg to about 400 mg/kg.

37. The method of any one of the previous claims, wherein duration of Zika viremia in the subject and/or fetus is shortened.

38. The method of any one of the previous claims, wherein the Zika viral load in the blood and/or a tissue of the subject and/or fetus is prevented or decreased.

39. The method of claim 38, wherein the Zika viral load is decreased by at least 25%, at least 50%, at least 75%, at least 85%, at least 90%, at least 95%, at least 99%, or 100%.

40. The method of claim 38, wherein the Zika viral load in the blood is decreased in the subject.

41. The method of claim 38, wherein the Zika viral load in the blood is prevented or decreased in the fetus.

42. The method of claim 38, wherein the Zika viral load in a tissue in the subject is decreased.

43. The method of claim 38, wherein Zika viral load in a tissue in the fetus is prevented or decreased.

44. The method of claim 2, 3, 38, 42 or 43, wherein the tissue is selected from the group consisting of brain, dura mater, spinal cord, sciatic nerve, cochlea, cerebrum, cerebellum, aqueous humor, optic nerve, sclera, cornea, retina, pericardium, heart, aorta, lung, seminal vesicle, prostate/uterus, testis, ovary, articular cartilage, adipose tissue-omentus, epidermis/dermis of abdomen, muscle-quadriceps, bone marrow, tonsil, spleen, thymus, lymph nodes, gastric contents, esophagus, stomach, duodenum, jejunum, ileum, cecum, colon, bile aspirate, liver, meconium, tongue, urinary bladder, kidney, urine, thyroid, adrenal gland, pituitary, pancreas, fetal blood, placental disk, uterus, decidua, amniotic/chorionic membrane, amniotic fluid, umbilical cord, cord blood, or any combination thereof.
